(19) United States
(12) Patent Application Publication
(10) Pub. No.: US 2026/0335349 A1
(43) Pub. Date: Oct. 29, 2026

(54) INTRAVESICAL SYSTEMS AND GEMCITABINE RELEASE RATES FOR TREATING BLADDER CANCER

(71) Applicant: Janssen Biotech, Inc., Horsham, PA (US)

(72) Inventors: Hussein SWEITI, Royersford, PA (US); Christopher J. CUTIE, Lexington, MA (US); Kiran A. PATEL, Chesterbrook, PA (US)

(21) Appl. No.: 19/636,273

(22) Filed: Apr. 1, 2026

Related U.S. Application Data
(60) Provisional application No. 63/782,384, filed on Apr. 2, 2025.
(60) Provisional application No. 63/794,659, filed on Apr. 25, 2025.
(60) Provisional application No. 63/851,293, filed on Jul. 25, 2025.
(60) Provisional application No. 63/878,843, filed on Sep. 9, 2025.
(60) Provisional application No. 63/896,160, filed on Oct. 9, 2025.
(60) Provisional application No. 63/998,659, filed on Mar. 6, 2026.

(51) Int. Cl. A61K 31/7068; A61K 9/00; A61K 9/20; A61K 47/10; A61K 47/16; A61K 47/22; A61K 47/34; A61P 35/00

(52) U.S. Cl. CPC A61K 31/7068; A61K 9/0034; A61K 9/2072; A61K 47/10; A61K 47/16; A61K 47/22; A61K 47/34; A61P 35/00

(57) ABSTRACT

The present disclosure relates generally to methods of treating bladder cancer using intravesical delivery systems comprising gemcitabine. More specifically, the present disclosure relates to methods of treating non-muscle invasive bladder cancer, such as Bacillus Calmette Guérin (BCG)-unresponsive High-Risk, non-muscle invasive bladder cancer (HR-NMIBC) with carcinoma in situ (CIS) with or without papillary tumors.

### CROSS-REFERENCE TO RELATED APPLICATIONS

This application claims priority to and the benefit of U.S. Provisional Application No. 63/782,384, filed Apr. 2, 2025; U.S. Provisional Application No. 63/794,659, filed Apr. 25, 2025; U.S. Provisional Application No. 63/851,293, filed Jul. 25, 2025; U.S. Provisional Application No. 63/878,843, filed Sep. 9, 2025; U.S. Provisional Application No. 63/896,160, filed Oct. 9, 2025; and U.S. Provisional Application No. 63/998,659, filed Mar. 6, 2026, the contents of each of which are incorporated herein by reference in their entirety.

### FIELD

The present disclosure relates generally to methods of treating bladder cancer. More specifically, the present disclosure relates to methods of treating non-muscle invasive bladder cancer, such as Bacillus Calmette Guérin (BCG)-unresponsive High-Risk, non-muscle invasive bladder cancer (HR-NMIBC) with carcinoma in situ (CIS) with or without papillary tumors. Also described herein are intravesical systems for treating bladder cancer, as well as kits comprising an intravesical system with label listing contents and/or instructions for use.

### BACKGROUND

Bladder cancer is a significant medical problem, and currently available treatment options are unsatisfactory for a number of reasons. According to the Pharma Intelligence database (Citeline), as of the filing date of this application, there had been more than 1,300 clinical trials looking to identify safe and effective treatments for bladder cancer. Of those trials, more than 1,000 either failed to meet their outcome, were terminated, or were otherwise discontinued. There remains a significant unmet medical need for patients with bladder cancer and the subject matter described and claimed herein is a significant step to helping meet that need.

Most patients with high-risk non-muscle invasive bladder cancer (HR-NMIBC) experience disease recurrence (12%-78%) or progression (≤1%-46%) after standard of care transurethral resection of bladder tumor (TURBT) and intravesical bacillus Calmette-Guérin (BCG) treatment, often leading to BCG-unresponsive disease.

Current standard of care for BCG-unresponsive high-risk NMIBC is radical cystectomy. Radical cystectomy is a life-changing surgery associated with considerable morbidity (50%-70%) and significant impact on quality of life. Radical cystectomy is also associated with 90-day mortality risk (3%-5%), particularly in elderly patients (≤15%). Therefore, many patients are unable or unwilling to undergo this procedure, and real-world rates of radical cystectomy for NMIBC are low (<20%). US Food and Drug Administration (FDA)-approved treatment options for BCG-unresponsive carcinoma in situ include systemic pembrolizumab, intravesical nadofaragene firadenovec-vncg, and intravesical nogapendekin alfa inbakicept-pmln plus BCG. However, these treatments are associated with limited complete response rates (e.g., 41% with pembrolizumab, 51% with nadofaragene firadenovec, and 62% with nogapendekin alfa inbakicept+BCG) and response durability, systemic or immune-related toxicities, frequent urethral catheterizations, reliance on BCG as combination therapy, and limited physician adoption. These challenges underscore the persistent need for tolerable, effective bladder-sparing therapies in this population. Additionally, there are no approved treatments for BCG-unresponsive papillary disease-only high-risk NMIBC.

Thus, there exists a significant clinical need for alternative treatment options such as those that allow individuals to retain their bladders. The present application addresses these and other needs.

The disclosures of all publications, patents, patent applications and published patent applications referred to herein are hereby incorporated herein by reference in their entirety.

### SUMMARY

Described herein are methods of treating bladder cancer in an individual.

In some aspects, described herein are methods of treating bladder cancer in an individual comprising inserting an intravesical system comprising about 256 mg of gemcitabine HCl into the bladder of the individual once about every three weeks for at least 24 weeks, wherein each intravesical system is removed from the bladder about three weeks after insertion into the bladder and wherein each intravesical system is removed prior to insertion of another intravesical system, wherein each intravesical system provides sustained release of gemcitabine into bladder urine, wherein about 65% to about 85% of the gemcitabine is released over about the first 7 days after insertion into the bladder and about 85% to about 100% of the gemcitabine is released over about 21 days after insertion into the bladder, and wherein the intravesical system comprises gemcitabine minitablets comprising a total of: i) about 256 mg gemcitabine HCl; ii) about 10 mg to about 20 mg Povidone 30; iii) about 5 mg to about 12 mg polyethylene glycol (PEG) 8000; and iv) about 35 mg to about 50 mg urea.

In some embodiments, about 70% of the gemcitabine is released over about the first 7 days after insertion into the bladder and about 90% of the gemcitabine is released over about 21 days after insertion into the bladder, and wherein the percent of gemcitabine released is estimated using an in vitro release model. In some embodiments, about 76.9% of the gemcitabine is released over about the first 7 days after insertion into the bladder and about 99% of the gemcitabine is released over about 21 days after insertion into the bladder, and wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some aspects, described herein are methods of delivering gemcitabine to an individual having bladder cancer, the method comprising inserting an intravesical system comprising about 256 mg gemcitabine HCL into the bladder of the individual to target a pharmacokinetic (PK) profile for gemcitabine, wherein the PK profile has: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose; and wherein the intravesical system comprises gemcitabine minitablets comprising a total of: i) about 256 mg gemcitabine HCL; ii) about 10 mg to about 20 mg Povidone 30; iii) about 5 mg to about 12 mg polyethylene glycol (PEG) 8000; and iv) about 35 mg to about 50 mg urea.

In some embodiments, the individual has non-muscle invasive bladder cancer (NMIBC). In some embodiments, the individual has Bacillus Calmette Guerin (BCG)-unresponsive, high-risk, non-muscle invasive bladder cancer (HR-NMIBC) with carcinoma in situ (CIS) with or without papillary tumors. In some embodiments, each intravesical system produces a systemic exposure of gemcitabine in the individual of less than about 0.1 μg/mL. In some embodiments, each intravesical system produces a maximum mean amount of about 31.5 mg gemcitabine related components (GRC) in urine on Day 3.

In some embodiments, each intravesical system releases about 2% to about 20% of the gemcitabine on each of days 1-7 after the intravesical system is inserted into the bladder of the individual. In some embodiments, each intravesical system produces a PK profile having a mean excreted GRC of about 2 mg to about 21 mg on day 1 after insertion in the bladder, about 17 mg to about 37 mg on day 2 after insertion in the bladder, about 20 mg to about 40 mg on day 3 after insertion into the bladder, about 12 mg to about 38 mg on day 4 after insertion into the bladder, about 5 mg to about 35 mg on day 5 after insertion into the bladder, about 2 mg to about 27 mg on day 6 after insertion into the bladder, and/or about 1 mg to about 20 mg on day 7 after insertion into the bladder. In some embodiments, each intravesical system produces an average urine concentration of about 2 μg/mL to about 15 μg/mL on about days 1-7 after insertion into the bladder of the individual.

In some embodiments, each gemcitabine minitablet comprises: i) about 19 mg to about 24 mg gemcitabine HCL; ii) about 0.5 mg to about 1.5 mg Povidone 30; iii) about 0.4 mg to about 1 mg polyethylene glycol (PEG) 8000; and iv) about 2 mg to about 5 mg urea. In some embodiments, each gemcitabine minitablet comprises: i) about 21.36 mg gemcitabine HCL; ii) about 1.12 mg Povidone 30; iii) about 0.67 mg polyethylene glycol (PEG) 8000; and iv) about 3.55 mg urea. In some embodiments, each gemcitabine minitablet comprises: i) about 72% to about 89% w/w gemcitabine HCL; ii) about 1.6% to about 6.6% w/w Povidone 30; iii) about 1.3% to about 4.4% w/w polyethylene glycol (PEG) 8000; and iv) about 7% to about 20% w/w urea. In some embodiments, each gemcitabine minitablet comprises: i) about 80% w/w gemcitabine HCL; ii) about 4.2% w/w Povidone 30; iii) about 2.5% w/w polyethylene glycol (PEG) 8000; and iv) about 13.3% w/w urea. In some embodiments, the gemcitabine minitablets of each intravesical system comprise a total of: i) about 256.3 mg gemcitabine HCL; ii) about 13.4 mg Povidone 30; iii) about 8 mg polyethylene glycol (PEG) 8000; and iv) about 42.6 mg urea.

In some embodiments, each intravesical system comprises osmotic minitablets comprising: i) about 15 mg to about 25 mg urea; and ii) about 1 mg to about 4 mg polyethylene oxide (PEO). In some embodiments, each intravesical system comprises osmotic minitablets comprising: i) about 21.6 mg urea; and ii) about 2.4 mg polyethylene oxide (PEO). In some embodiments, each intravesical system comprises osmotic minitablets comprising: i) about 79% to about 96% w/w urea; and ii) about 4% to about 20% w/w polyethylene oxide (PEO). In some embodiments, each intravesical system comprises osmotic minitablets comprising: i) about 90% w/w urea; and ii) about 10% w/w polyethylene oxide (PEO). In some embodiments, the osmotic minitablets of each intravesical system comprise a total of: i) about 630 mg to about 670 mg urea; and ii) about 50 mg to about 90 mg polyethylene oxide (PEO). In some embodiments, the osmotic minitablets of each intravesical system comprise a total of: i) about 648 mg urea; and ii) about 72 mg polyethylene oxide (PEO). In some embodiments, the osmotic minitablets further comprise colorant. In some embodiments, the colorant is FD&C Blue No. 1 colorant.

In some embodiments, each intravesical system comprises a flexible bi-oval shaped tube. In some embodiments, when in the retention shape, the intravesical system defines a bi-oval coiled shape having a maximum width and a maximum height perpendicular to the maximum width, wherein the maximum width is about 4-7 cm and the maximum height is about 3-6 cm. In some embodiments, the maximum width is about 5.5 cm and the maximum height is about 4.5 cm. In some embodiments, each intravesical system comprises 12 gemcitabine minitablets and 30 osmotic agent minitablets.

In some embodiments, each intravesical system comprises: a) 12 gemcitabine minitablets comprising a total of: i) about 256.3 mg gemcitabine HCL; ii) about 13.4 mg Povidone 30; iii) about 8 mg polyethylene glycol (PEG) 8000; and iv) about 42.6 mg urea; and b) 30 osmotic agent minitablets comprising a total of: i) about 648 mg urea; and ii) about 72 mg polyethylene oxide (PEO).

In some embodiments, each intravesical system comprises an orifice with an about 25 micron to about 300 micron diameter. In some embodiments, each intravesical system comprises an orifice with an about 150 micron diameter. In some embodiments, each intravesical system is configured to be freely mobile in the bladder of the individual.

In some embodiments, the individual is ineligible for or has elected not to undergo radical cystectomy.

In some aspects, described herein are methods of treating Bacillus Calmette Guerin (BCG)-unresponsive, high-risk, non-muscle invasive bladder cancer (HR-NMIBC) with carcinoma in situ (CIS) with or without papillary tumors in an individual, the method comprising inserting an intravesical system comprising about 256 mg of gemcitabine hydrochloride into the bladder once every 3 weeks up to 6 months (8 doses), followed by once every 12 weeks for up to 18 months, or until persistent or recurrent HR-NMIBC, disease progression, or unacceptable toxicity. In some embodiments, the method comprises a total of 14 doses over 2 years of therapy.

In some aspects, described herein is an intravesical system comprising: a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 256 mg gemcitabine HCL; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

In some aspects, described herein is an intravesical system comprising gemcitabine minitablets comprising a total of: i) about 256 mg gemcitabine HCL; ii) about 10 mg to about 20 mg Povidone 30; iii) about 5 mg to about 12 mg polyethylene glycol (PEG) 8000; and iv) about 35 mg to about 50 mg urea, wherein, when tested using an in vitro release model, the intravesical system releases about 70% of the gemcitabine over about 7 days and about 90% of the gemcitabine about 21 days.

In some embodiments, when the intravesical system is indwelling in the bladder of an individual, the intravesical system generates a pharmacokinetic (PK) profile having: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose.

In some embodiments, the intravesical system further comprises spacers at each end of the housing. In some embodiments, an adhesive secures the spacers within the ends of the drug reservoir lumen.

In some embodiments, the gemcitabine minitablets comprise a total of: i) about 256.3 mg gemcitabine HCl; ii) about 10 mg to about 20 mg Povidone 30; iii) about 5 mg to about 12 mg polyethylene glycol (PEG) 8000; and iv) about 35 mg to about 50 mg urea. In some embodiments, the gemcitabine minitablets comprise a total of: i) about 256.3 mg gemcitabine HCl; ii) about 13.4 mg Povidone 30; iii) about 8 mg polyethylene glycol (PEG) 8000; and iv) about 42.6 mg urea. In some embodiments, the osmotic minitablets comprise a total of: i) about 630 mg to about 670 mg urea; and ii) about 50 mg to about 90 polyethylene oxide (PEO). In some embodiments, the osmotic minitablets comprise a total of: i) about 648 mg urea; and ii) about 72 mg polyethylene oxide (PEO). In some embodiments, the osmotic minitablets further comprise FD&C Blue No. 1 colorant.

In some embodiments, the intravesical system is configured as a flexible bi-oval shaped tube. In some embodiments, when in the retention shape, the intravesical system defines a bi-oval coiled shape having a maximum width and a maximum height perpendicular to the maximum width, wherein the maximum width is about 4-7 cm and the maximum height is about 3-6 cm. In some embodiments, the maximum width is about 5.5 cm and the maximum height is about 4.5 cm. In some embodiments, the intravesical system is sterile. In some embodiments, the intravesical system is configured to freely mobile in a bladder of an individual.

In some aspects, described herein are methods of treating bladder cancer in an individual, wherein such treatment results in a) a complete response (CR) rate of about 74% to about 91% in a population of patients who have received such treatment; b) a median duration of response (DoR) of about 9 months to about 30 months in a population of patients who have received such treatment; c) a 12-month complete response (CR) rate of about 40% to about 50% in a population of patients who have received such treatment; and/or d) a 12-month duration of response (DoR) rate of about 43% to about 67% in a population of patients who have received such treatment.

In some embodiments, such treatment results in a) a complete response (CR) rate of about 84% in a population of patients who have received such treatment; b) a median duration of response (DoR) of about 25.8 months in a population of patients who have received such treatment; c) a 12-month complete response (CR) rate of about 45.9% in a population of patients who have received such treatment; d) a 12-month duration of response (DoR) rate of about 55.7% in a population of patients who have received such treatment; and/or e) a 12-month duration of response (DoR) rate of about 52.9% in a population of patients who have received such treatment.

In some aspects, described herein are methods of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a median duration of response (DoR) of about 9 months to about 30 months in a population of patients who have received such treatment. In some embodiments, the median DoR is about 25.8 months.

In some aspects, described herein are methods of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a 12-month complete response (CR) rate of about 40% to about 50% in a population of patients who have received such treatment. In some embodiments, the 12-month CR rate is about 45.9%.

In some aspects, described herein are methods of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a 12-month duration of response (DoR) rate of about 43% to about 67% in a population of patients who have received such treatment. In some embodiments, the 12-month DoR rate is about 55.7%. In some embodiments, the 12-month DoR rate is about 52.9%.

In some aspects, described herein are methods of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a median time to onset of response of about 2 months to about 9 months in a population of patients who have received such treatment. In some embodiments, the median time to onset of response is about 2.8 months.

In some embodiments, the methods described herein further comprise inserting an intravesical system comprising about 256 mg of gemcitabine HCL into the bladder of the individual once every twelve weeks for up to 18 months.

In some aspects, described herein are kits comprising an intravesical system and instructions for use to treat Bacillus Calmette Guerin (BCG)-unresponsive, high-risk, non-muscle invasive bladder cancer (HR-NMIBC) with carcinoma in situ (CIS) with or without papillary tumors, the intravesical system comprising a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 256 mg gemcitabine HCL; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

In some embodiments, the kit further comprises a urinary placement catheter adapted for insertion of the intravesical system into the bladder. In some embodiments, the intravesical system is sterile.

In some embodiments, each intravesical system produces a mean urinary excretion on Days 2 through 5 of 21 mg to 32 mg per day as gemcitabine and dFdU. In some embodiments, each intravesical system comprises an almost white to light pink-brown colored gemcitabine component at the center surrounded on each side by off white to light blue-colored osmotic components.

In some embodiments, the intravesical system comprises an almost white to light pink-brown colored gemcitabine component at the center surrounded on each side by off white to light blue-colored osmotic components.

In some aspects, described herein is a method of treating bladder cancer in an individual comprising inserting an intravesical system comprising about 225 mg of gemcitabine into the bladder of the individual once about every three weeks for at least 24 weeks, wherein each intravesical system is removed from the bladder about three weeks after insertion into the bladder and wherein each intravesical system is removed prior to insertion of another intravesical system, wherein each intravesical system provides sustained release of gemcitabine into bladder urine, wherein about 65% to about 85% of the gemcitabine is released over about the first 7 days after insertion into the bladder and about 85% to about 100% of the gemcitabine is released over about 21 days after insertion into the bladder, and wherein the intravesical system comprises gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

In some embodiments, about 70% of the gemcitabine is released over about the first 7 days after insertion into the bladder and about 90% of the gemcitabine is released over about 21 days after insertion into the bladder, and wherein the percent of gemcitabine released is estimated using an in vitro release model. In some embodiments, about 76.9% of the gemcitabine is released over about the first 7 days after insertion into the bladder and about 99% of the gemcitabine is released over about 21 days after insertion into the bladder, and wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some aspects, described herein is a method of delivering gemcitabine to an individual having bladder cancer, the method comprising inserting an intravesical system comprising about 225 mg gemcitabine into the bladder of the individual to target a pharmacokinetic (PK) profile for gemcitabine, wherein the PK profile has: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose; and wherein the intravesical system comprises gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

In some embodiments, the individual has non-muscle invasive bladder cancer (NMIBC), optionally wherein the individual has Bacillus Calmette Guerin (BCG)-unresponsive NMIBC with carcinoma in situ (CIS) with or without papillary tumors. In some embodiments, the individual has Bacillus Calmette Guerin (BCG)-unresponsive, high-risk, non-muscle invasive bladder cancer (HR-NMIBC) with carcinoma in situ (CIS) with or without papillary tumors.

In some embodiments, each intravesical system produces a systemic exposure of gemcitabine in the individual of less than about 0.1 μg/mL. In some embodiments, each intravesical system produces a maximum mean amount of about 31.5 mg gemcitabine related components (GRC) in urine on Day 3. In some embodiments, each intravesical system releases about 2% to about 20% of the gemcitabine on each of days 1-7 after the intravesical system is inserted into the bladder of the individual. In some embodiments, each intravesical system produces a PK profile having a mean excreted GRC of about 2 mg to about 21 mg on day 1 after insertion in the bladder, about 17 mg to about 37 mg on day 2 after insertion in the bladder, about 20 mg to about 40 mg on day 3 after insertion into the bladder, about 12 mg to about 38 mg on day 4 after insertion into the bladder, about 5 mg to about 35 mg on day 5 after insertion into the bladder, about 2 mg to about 27 mg on day 6 after insertion into the bladder, and/or about 1 mg to about 20 mg on day 7 after insertion into the bladder. In some embodiments, each intravesical system produces an average urine concentration of about 2 μg/mL to about 15 μg/mL on about days 1-7 after insertion into the bladder of the individual.

In some embodiments, each gemcitabine minitablet comprises: i) about 19 mg to about 24 mg gemcitabine HCL; ii) about 0.5 mg to about 1.5 mg Povidone 30; iii) about 0.4 mg to about 1 mg polyethylene glycol (PEG) 8000; and iv) about 2 mg to about 5 mg urea. In some embodiments, each gemcitabine minitablet comprises: i) about 21.36 mg gemcitabine HCL; ii) about 1.12 mg Povidone 30; iii) about 0.67 mg polyethylene glycol (PEG) 8000; and iv) about 3.55 mg urea. In some embodiments, each gemcitabine minitablet comprises: i) about 72% to about 89% w/w gemcitabine HCL; ii) about 1.6% to about 6.6% w/w Povidone 30; iii) about 1.3% to about 4.4% w/w polyethylene glycol (PEG) 8000; and iv) about 7% to about 20% w/w urea. In some embodiments, each gemcitabine minitablet comprises: i) about 80% w/w gemcitabine HCL; ii) about 4.2% w/w Povidone 30; iii) about 2.5% w/w polyethylene glycol (PEG) 8000; and iv) about 13.3% w/w urea. In some embodiments, the gemcitabine minitablets of each intravesical system comprise a total of: i) about 225 mg gemcitabine; ii) about 13.4 mg Povidone 30; iii) about 8 mg polyethylene glycol (PEG) 8000; and iv) about 42.6 mg urea.

In some embodiments, each intravesical system comprises osmotic minitablets comprising: i) about 15 mg to about 25 mg urea; and ii) about 1 mg to about 4 mg polyethylene oxide (PEO). In some embodiments, each intravesical system comprises osmotic minitablets comprising: i) about 21.6 mg urea; and ii) about 2.4 mg polyethylene oxide (PEO). In some embodiments, each intravesical system comprises osmotic minitablets comprising: i) about 79% to about 96% w/w urea; and ii) about 4% to about 20% w/w polyethylene oxide (PEO). In some embodiments, each intravesical system comprises osmotic minitablets comprising: i) about 90% w/w urea; and ii) about 10% w/w polyethylene oxide (PEO). In some embodiments, the osmotic minitablets of each intravesical system comprise a total of: i) about 630 mg to about 670 mg urea; and ii) about 50 mg to about 90 mg polyethylene oxide (PEO). In some embodiments, the osmotic minitablets of each intravesical system comprise a total of: i) about 648 mg urea; and ii) about 72 mg polyethylene oxide (PEO). In some embodiments, the osmotic minitablets further comprise colorant. In some embodiments, the colorant is FD&C Blue No. 1 colorant.

In some embodiments, each intravesical system comprises a flexible bi-oval shaped tube. In some embodiments, when in the retention shape, the intravesical system defines a bi-oval coiled shape having a maximum width and a maximum height perpendicular to the maximum width, wherein the maximum width is about 4-7 cm and the maximum height is about 3-6 cm. In some embodiments, the maximum width is about 5.5 cm and the maximum height is about 4.5 cm.

In some embodiments, each intravesical system comprises 12 gemcitabine minitablets and 30 osmotic agent minitablets. In some embodiments, each intravesical system comprises: a) 12 gemcitabine minitablets comprising a total of: i) about 225 mg gemcitabine; ii) about 13.4 mg Povidone 30; iii) about 8 mg polyethylene glycol (PEG) 8000; and iv) about 42.6 mg urea; and b) 30 osmotic agent minitablets comprising a total of: i) about 648 mg urea; and ii) about 72 mg polyethylene oxide (PEO). In some embodiments, each intravesical system comprises an orifice with an about 25 micron to about 300 micron diameter. In some embodiments, each intravesical system comprises an orifice with an about 150 micron diameter. In some embodiments, each intravesical system is configured to be freely mobile in the bladder of the individual. In some embodiments, the individual is ineligible for or has elected not to undergo radical cystectomy.

In some aspects, described herein is a method of treating Bacillus Calmette Guerin (BCG)-unresponsive, non-muscle invasive bladder cancer (NMIBC) with carcinoma in situ (CIS) with or without papillary tumors in an individual, the method comprising inserting an intravesical system comprising about 225 mg of gemcitabine into the bladder once every 3 weeks up to 6 months (8 doses), followed by once every 12 weeks for up to 18 months, or until persistent or recurrent NMIBC, disease progression, or unacceptable toxicity. In some embodiments, the method comprises a total of 14 doses over 2 years of therapy.

In some aspects, described herein is an intravesical system comprising: a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 225 mg gemcitabine; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

In some aspects, described herein is an intravesical system comprising gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea, wherein, when tested using an in vitro release model, the intravesical system releases about 70% of the gemcitabine over about 7 days and about 90% of the gemcitabine about 21 days.

In some embodiments, when the intravesical system is indwelling in the bladder of an individual, the intravesical system generates a pharmacokinetic (PK) profile having: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose.

In some embodiments, the intravesical system further comprising spacers at each end of the housing. In some embodiments, an adhesive secures the spacers within the ends of the drug reservoir lumen.

In some embodiments, the gemcitabine minitablets comprise a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea. In some embodiments, the gemcitabine minitablets comprise a total of: i) about 225 mg gemcitabine; ii) about 13.4 mg Povidone 30; iii) about 8 mg polyethylene glycol (PEG) 8000; and iv) about 42.6 mg urea. In some embodiments, the osmotic minitablets comprise a total of: i) about 630 mg to about 670 mg urea; and ii) about 50 mg to about 90 polyethylene oxide (PEO). In some embodiments, the osmotic minitablets comprise a total of: i) about 648 mg urea; and ii) about 72 mg polyethylene oxide (PEO). In some embodiments, the osmotic minitablets further comprise FD&C Blue No. 1 colorant.

In some embodiments, the intravesical system is configured as a flexible bi-oval shaped tube. In some embodiments, when in the retention shape, the intravesical system defines a bi-oval coiled shape having a maximum width and a maximum height perpendicular to the maximum width, wherein the maximum width is about 4-7 cm and the maximum height is about 3-6 cm. In some embodiments, the maximum width is about 5.5 cm and the maximum height is about 4.5 cm. In some embodiments, the intravesical system is sterile. In some embodiments, the intravesical system is configured to freely mobile in a bladder of an individual.

In some embodiments, treatment with the methods described herein results in a complete response (CR) rate of about 72% to about 90% in a population of patients who have received such treatment; a median duration of response (DoR) of about 9 months to about 30 months in a population of patients who have received such treatment; a 12-month complete response (CR) rate of about 40% to about 50% in a population of patients who have received such treatment; and/or a 12-month duration of response (DoR) rate of about 43% to about 67% in a population of patients who have received such treatment.

In some embodiments, such treatment results in a complete response (CR) rate of about 82% in a population of patients who have received such treatment; a median duration of response (DoR) of about 25.8 months in a population of patients who have received such treatment; a 12-month complete response (CR) rate of about 45.9% in a population of patients who have received such treatment; and/or a 12-month duration of response (DoR) rate of about 51% in a population of patients who have received such treatment.

In some aspects, described herein is a method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a median duration of response (DoR) of about 9 months to about 30 months in a population of patients who have received such treatment. In some embodiments, the median DoR is about 25.8 months.

In some aspects, described herein is a method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a 12-month complete response (CR) rate of about 40% to about 50% in a population of patients who have received such treatment. In some embodiments, the 12-month CR rate is about 45.9%.

In some aspects, described herein is a method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a 12-month duration of response (DoR) rate of about 43% to about 67% in a population of patients who have received such treatment. In some embodiments, the 12-month DoR rate is about 51%.

In some aspects, described herein is a method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a median time to onset of response of about 2 months to about 9 months in a population of patients who have received such treatment. In some embodiments, the median time to onset of response is about 2.8 months.

In some embodiments, the method further comprises inserting an intravesical system comprising about 225 mg of gemcitabine into the bladder of the individual once every twelve weeks for up to 18 months.

In some aspects, described herein is a kit comprising an intravesical system and instructions for use to treat Bacillus Calmette Guerin (BCG)-unresponsive, non-muscle invasive bladder cancer (NMIBC) with carcinoma in situ (CIS) with or without papillary tumors, the intravesical system comprising a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 225 mg gemcitabine; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

In some embodiments, the kit further comprises a urinary placement catheter adapted for insertion of the intravesical system into the bladder. In some embodiments, the intravesical system is sterile. In some embodiments, each intravesical system produces a mean urinary excretion on Days 2 through 5 of 21 mg to 32 mg per day as gemcitabine and dFdU.

In some embodiments, each intravesical system comprises an almost white to light pink-brown colored gemcitabine component at the center surrounded on each side by off white to light blue-colored osmotic components. In some embodiments, the intravesical system comprises an almost white to light pink-brown colored gemcitabine component at the center surrounded on each side by off white to light blue-colored osmotic components.

In some aspects, described herein is a method of treating bladder cancer in an individual comprising administering to the individual an approved drug product comprising gemcitabine. In some embodiments, the approved drug product is described by the approved drug product label of Example 9.

In some embodiments, such treatment results in a complete response (CR) rate of about 72% to about 90% in a population of patients who have received such treatment; a median duration of response (DoR) of about 9 months to about 30 months in a population of patients who have received such treatment; a 12-month complete response (CR) rate of about 40% to about 50% in a population of patients who have received such treatment; and/or a 12-month duration of response (DoR) rate of about 43% to about 67% in a population of patients who have received such treatment.

In some embodiments, such treatment results in a complete response (CR) rate of about 82% in a population of patients who have received such treatment; a median duration of response (DoR) of about 25.8 months in a population of patients who have received such treatment; a 12-month complete response (CR) rate of about 45.9% in a population of patients who have received such treatment; and/or a 12-month duration of response (DoR) rate of about 51% in a population of patients who have received such treatment.

In some aspects, described herein is a method of improving complete response (CR) rate in a population of individuals, the method comprising administering to the population of individuals an approved drug product comprising gemcitabine. In some embodiments, the CR rate is about 82%.

In some aspects, described herein is a method of improving median duration of response in a population of individuals, the method comprising administering to the population of individuals an approved drug product comprising gemcitabine. In some embodiments, the median duration of response is about 25.8 months.

In some aspects, described herein is a method of improving 12-month complete response (CR) rate in a population of individuals, the method comprising administering to the population of individuals an approved drug product comprising gemcitabine. In some embodiments, the 12-month CR rate is about 45.9%.

In some aspects, described herein is a method of improving 12-month duration of response (DoR) rate in a population of individuals, the method comprising administering to the population of individuals an approved drug product comprising gemcitabine. In some embodiments, the 12-month DoR rate is about 51%.

In some embodiments, the method comprises inserting an intravesical system comprising about 225 mg of gemcitabine into the bladder once every 3 weeks up to 6 months (8 doses), followed by once every 12 weeks for up to 18 months (6 doses), or until persistent or recurrent NMIBC, disease progression, or unacceptable toxicity. In some embodiments, each intravesical system is removed after a 3-week indwelling period.

In some aspects, described herein is a kit comprising: an intravesical device comprising 225 mg gemcitabine, co-packaged with a urinary catheter and stylet, and instructions for use according to any of the methods described herein, or example 10.

In some aspects, described herein is a method of treating bladder cancer in an individual comprising inserting an intravesical system comprising about 225 mg of gemcitabine into the bladder of the individual once about every three weeks for at least 24 weeks, wherein each intravesical system is removed from the bladder about three weeks after insertion into the bladder and wherein each intravesical system is removed prior to insertion of another intravesical system, wherein each intravesical system provides sustained release of gemcitabine into bladder urine to target a pharmacokinetic (PK) profile for gemcitabine, wherein the PK profile has a mean urinary excretion on Days 2 through 5 of 21 mg to 32 mg per day as gemcitabine and dFdU, wherein about 65% to about 85% of the gemcitabine is released over about the first 7 days after insertion into the bladder and about 85% to about 100% of the gemcitabine is released over about 21 days after insertion into the bladder, and wherein the intravesical system comprises gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

In some embodiments, about 77% of the gemcitabine is released over about the first 7 days after insertion into the bladder and about 99% of the gemcitabine is released over about 21 days after insertion into the bladder, and wherein the percent of gemcitabine released is estimated using in vivo data comprising urine gemcitabine related component (GRC) amount-time profiles, and wherein the percent of gemcitabine released is estimated using nonlinear mixed effect modeling. In some embodiments, the about 225 mg of gemcitabine comprises about 256.3 mg of gemcitabine HCL.

### BRIEF DESCRIPTION OF THE DRAWINGS

The present application can be best understood by reference to the following description taken in conjunction with the accompanying figures included in the specification.

FIGS. 1A-1D show an exemplary intravesical system, TAR-200, in accordance with some aspects. FIG. 1A is a top view illustrating the intravesical system in a bi-oval retention shape, in accordance with some aspects. FIG. 1B is an enlarged, top view of the silicone spacers and silicone adhesive at the ends of the housing of the intravesical system of FIG. 1A, in accordance with some aspects. FIG. 1C illustrates a cross-sectional view of the intravesical system along line C-C in FIG. 1A, in accordance with some aspects. FIG. 1D is a schematic, side view depicting the intravesical system of FIGS. 1A-1C in its elongated, uncoiled form, in accordance with some aspects.

FIGS. 2A-2B show an exemplary insertion device for use with the intravesical system provided herein, TAR-200, for the transurethral placement of the intravesical system into the bladder. FIG. 2A shows a side view of a first embodiment of the exemplary insertion device.

FIG. 2B shows a top view of another embodiment of the exemplary insertion device. The insertion devices may be referred to as a urological placement catheter (Urinary Placement Catheter (UPC) or Inserter. The UPC comprises a catheter and stylet, as shown in FIGS. 2A and 2B. In FIG. 2B, a close-up view is shown to highlight the depth markings present on the catheter shaft.

FIGS. 3A-3F are pictures of an exemplary sequence of loading TAR-200 into the Urinary Placement Catheter and deploying it therefrom. In FIG. 3A, TAR-200 is depicted next to the Urinary Placement Catheter and in the retention shape, suited to retain the system within the bladder. FIG. 3B shows a healthcare provider manually uncoiling TAR-200 from its native bi-oval shape and loading TAR-200 into the catheter shaft of the Urinary Placement Catheter. FIG. 3C shows the healthcare provider inserting the stylet into the catheter shaft to advance TAR-200 through the catheter shaft. FIG. 3D shows the stylet manually advancing TAR-200 through the catheter shaft and TAR-200 beginning to emerge from an exit port of the catheter shaft. FIG. 3E shows TAR-200 as it emerges from the exit port of the catheter shaft as it is deployed into the bladder lumen (not depicted). FIG. 3F shows TAR-200 just prior to complete deployment from the catheter shaft into the bladder (not depicted), where TAR-200 returns to its original bi-oval form.

FIG. 4 shows the percentage of gemcitabine released from the intravesical system (TAR-200) over three weeks in vitro. The line represents an average from twelve intravesical systems.

FIG. 5 shows estimated gemcitabine urine concentrations over 7 days using TAR-200 delivery compared to instillation.

FIGS. 6A-6B show average urine concentration of gemcitabine in minipigs following transurethral intravesical deployment of TAR-200 for 7 days (FIG. 6A) and 28 days (FIG. 6B), respectively.

FIGS. 7A-7B show the study design for an open-label, parallel-group, multicenter study of the efficacy and safety of intravesical TAR-200 or systemic cetrelimab in participants with Bacillus Calmette Guérin (BCG)-unresponsive High-Risk, non-muscle invasive bladder cancer (HR-NMIBC) with carcinoma in situ (CIS) with or without papillary tumors in an individual. Dosing regimens for Year 1 are shown in FIG. 7A, and dosing regimens for Years 2 and 3 are shown in FIG. 7B.

FIGS. 8A-8F show complete response (CR) data from the study shown in FIGS. 7A-7B. FIG. 8A shows a summary of CR data from a September 2024 clinical cutoff date. FIG. 8B shows overall CR data from a February 2025 clinical cutoff date. FIG. 8C shows observed 12-month overall CR rate. FIG. 8D shows 12, 18, and 24-month Kaplan-Meier (KM) estimated overall CR rate from a February 2025 clinical cutoff date. FIG. 8E shows CR rate across patient subgroups. FIG. 8F shows 12 and 24-month Kaplan-Meier (KM) estimated overall CR rate from a March 2025 clinical cutoff date.

FIGS. 9A-9C show duration of response (DoR) data from the study shown in FIGS. 7A-7B. FIG. 9A shows a summary of DoR data from a September 2024 clinical cutoff date. FIG. 9B shows a swim lane plot of response events from a February 2025 clinical cutoff date. FIG. 9C shows a swim lane plot of response events from a March 2025 clinical cutoff date.

FIGS. 10A-10E show gemcitabine pharmacokinetic data from the study shown in FIGS. 7A-7B. FIG. 10A shows a summary of the data from a September 2024 cutoff date, organized by treatment group. FIG. 10B further segments the data from FIG. 10A. FIG. 10C shows a summary of the data from a September 2024 cutoff date, combining results collected from both treatment groups. FIG. 10D shows urine concentration of gemcitabine results over 1 week from a first gemcitabine dosing cycle. FIG. 10E shows urine concentration of gemcitabine results over 1 week from a second, third, or fourth dosing cycle (individuals provided urine samples for one of these dosing cycles).

FIGS. 11A-11E show dFdU pharmacokinetic data from the study shown in FIGS. 7A-7B. FIG. 11A shows a summary of the data from a September 2024 cutoff date, organized by treatment group. FIG. 11B further segments the data from FIG. 11A. FIG. 11C shows a summary of the data from a September 2024 cutoff date, combining results collected from both treatment groups. FIG. 11D shows dFdU urine concentration results over 1 week from a first gemcitabine dosing cycle. FIG. 11E shows dFdU urine concentration results over 1 week from a second, third, or fourth dosing cycle (individuals provided urine samples for one of these dosing cycles).

FIGS. 12A-12D show Gemcitabine Related Components (GRC, Gemcitabine+dFdU) pharmacokinetic data from the study shown in FIGS. 7A-7B. FIG. 12A shows a summary of the data from a September 2024 cutoff date, organized by treatment group. FIG. 12B shows a summary of the data from a September 2024 cutoff date, combining results collected from both treatment groups. FIG. 12C shows results of cumulative GRC excretion over 1 week from the first gemcitabine dosing cycle. FIG. 12D shows results of cumulative GRC excretion over 1 week from the second, third, or fourth dosing cycles (individuals provided urine samples for one of these dosing cycles).

FIG. 13 shows the amount of gemcitabine that is released from TAR-200 in vitro vs. in vivo, as measured in human urine each day for seven days. The percentage reported is the percentage of the initial gemcitabine contained in the TAR-200 system (225 mg free base) that was released each day.

FIGS. 14A-14E show the development of a population PK (popPK) model from the study shown in FIGS. 7A-7B. FIG. 14A shows the model structure. FIG. 14B shows a summary of the parameter estimates of the final popPK model. FIG. 14C shows the visual predictive check (VPC) of the model. The thin solid line represents the median, and the thin dashed lines 2 represent 5th and 95th percentiles of the observed values. The bolded solid line represents the median, and the bolded dashed lines represent 5th and 95th percentiles of the simulated values based on 1,000 simulations. The striped area represents the 95% confidence intervals of the median. The dotted areas represent the 5th and 95th percentiles of the simulated values. Circles represent the observed values. FIG. 14D shows a simulated typical profile of cumulative release. FIG. 14E shows observed daily release.

FIG. 15 shows safety data from the study shown in FIGS. 7A-7B.

FIG. 16 shows the estimated 12- and 24-month Duration of Response probabilities in patients who have achieved a complete response.

FIGS. 17A-17B show patient Quality of Life results after treatment with TAR-200. FIG. 17A shows Global Health Status (GHS). FIG. 17B shows Physical Functioning (PF).

FIG. 18 shows a summary of results for patients with HR NMIBC BCG-unresponsive CIS±papillary disease.

FIG. 19 shows goodness-of-fit plots of the final popPK model described in Example 7.

FIGS. 20A-20B show observed urine exposure versus nominal day within a dosage cycle, patterned by different creatinine clearance groups.

FIG. 21 shows a comparison of popPK model-predicted 7-day cumulative urine GRC amount between CR and non-CR Participants receiving TAR-200 alone.

FIG. 22 shows probability of CR versus quartile of popPK model-predicted 7-day cumulative GRC amount.

FIGS. 23A-23C show gemcitabine pharmacokinetic data from a study wherein individuals having muscle invasive bladder cancer (MIBC) received TAR-200 in combination with intravenous cetrelimab for three-week dosing cycles of gemcitabine. FIG. 23A shows a summary of the gemcitabine pharmacokinetic data from a September 2024 cutoff date. FIG. 23B shows urine concentration of gemcitabine results over 1 week from a first gemcitabine dosing cycle. FIG. 23C shows urine concentration of gemcitabine results over 1 week from a second or third dosing cycle (individuals provided urine samples for one of these dosing cycles).

FIGS. 24A-24C show dFdU pharmacokinetic data from a study wherein individuals having muscle invasive bladder cancer (MIBC) received TAR-200 in combination with intravenous cetrelimab for three-week dosing cycles of gemcitabine. FIG. 24A shows a summary of the dFdU pharmacokinetic data from a September 2024 cutoff date. FIG. 24B shows urine concentration of dFdU results over 1 week from a first gemcitabine dosing cycle.

FIG. 24C shows urine concentration of dFdU results over 1 week from a second or third dosing cycle (individuals provided urine samples for one of these dosing cycles).

FIGS. 25A-25C show GRC pharmacokinetic data from a study wherein individuals having muscle invasive bladder cancer (MIBC) received TAR-200 in combination with intravenous cetrelimab for three-week dosing cycles of gemcitabine. FIG. 25A shows a summary of the GRC pharmacokinetic data from a September 2024 cutoff date. FIG. 25B shows urine concentration of GRC results over 1 week from a first gemcitabine dosing cycle. FIG. 25C shows urine concentration of GRC results over 1 week from a second or third dosing cycle (individuals provided urine samples for one of these dosing cycles).

FIGS. 26A-26C show gemcitabine pharmacokinetic data from a study wherein individuals having muscle invasive bladder cancer (MIBC) received TAR-200 in combination with intravenous cetrelimab for three-week dosing cycles of gemcitabine before undergoing radical cystectomy. FIG. 26A shows a summary of the gemcitabine pharmacokinetic data from a May 2025 cutoff date. FIG. 26B shows urine concentration of gemcitabine results over 1 week from a first gemcitabine dosing cycle. FIG. 26C shows urine concentration of gemcitabine results over 1 week from a second or third dosing cycle (individuals provided urine samples for one of these dosing cycles).

FIGS. 27A-27C show dFdU pharmacokinetic data from a study wherein individuals having muscle invasive bladder cancer (MIBC) received TAR-200 in combination with intravenous cetrelimab for three-week dosing cycles of gemcitabine before undergoing radical cystectomy. FIG. 27A shows a summary of the dFdU pharmacokinetic data from a May 2025 cutoff date. FIG. 27B shows urine concentration of dFdU results over 1 week from a first gemcitabine dosing cycle. FIG. 27C shows urine concentration of dFdU results over 1 week from a second or third dosing cycle (individuals provided urine samples for one of these dosing cycles).

FIGS. 28A-28C show GRC pharmacokinetic data from a study wherein individuals having muscle invasive bladder cancer (MIBC) received TAR-200 in combination with intravenous cetrelimab for three-week dosing cycles of gemcitabine before undergoing radical cystectomy. FIG. 28A shows a summary of the GRC pharmacokinetic data from a May 2025 cutoff date. FIG. 28B shows urine concentration of GRC results over 1 week from a first gemcitabine dosing cycle. FIG. 28C shows urine concentration of GRC results over 1 week from a second or third dosing cycle (individuals provided urine samples for one of these dosing cycles).

FIG. 29 shows an illustration of the INLEXZO™ intravesical system, urinary catheter, and stylet, as they appear on the approved Instructions for Use.

FIG. 30 shows a close-up illustration of the INLEXZO™ intravesical system as it appears on the approved Instructions for Use.

FIG. 31 shows a close-up illustration of the urinary catheter and stylet as it appears on the approved Instructions for Use.

FIGS. 32A-32G show illustrations demonstrating instructions for preparing components for INLEXZO™ insertions, as they appear on the approved Instructions for Use. FIG. 32A shows preparation of two syringes with sterile water-based lubricant. FIG. 32B shows opening of the INLEXZO™ foil pouch. FIG. 32C shows examination of the white component pouches for damage. FIG. 32D shows opening of the white component pouches and transfer of the contents onto a sterile work surface. FIG. 32E shows the donning of sterile gloves. FIG. 32F shows the removal of plastic sleeves. FIG. 32G shows the inspection of components.

FIGS. 33A-33F show illustrations demonstrating instructions for insertion of INLEXZO™, as they appear on the approved Instructions for Use. FIG. 33A shows insertion of the urinary catheter without stylet. FIG. 33B shows injection of lubricant from the first syringe into the urinary catheter. FIG. 33C shows insertion of INLEXZO™ into the urinary catheter. FIG. 33D shows injection of lubricant from the second syringe into the urinary catheter. FIG. 33E shows insertion of the stylet into the urinary catheter. FIG. 33F shows removal of the urinary catheter and stylet together as a single unit.

FIG. 34 shows an illustration of providing the completed MRI Safety Information Card to the patient, as it appears on the approved Instructions for Use.

FIGS. 35A-35C show illustrations demonstrating instructions for removal of INLEXZO™ after the indwelling period, as they appear on the approved Instructions for Use. FIG. 35A shows insertion of the cystoscope. FIG. 35B shows grasping of INLEXZO™. FIG. 35C shows removal of INLEXZO™.

### DETAILED DESCRIPTION

Described herein are methods of treating bladder cancer using TAR-200, also known as INLEXZO™, an intravesical system comprising gemcitabine. TAR-200 (INLEXZO™) comprises about 225 mg gemcitabine and provides a sustained release of the gemcitabine into bladder urine during an indwelling period in the bladder.

TAR-200 is a novel intravesical system designed to provide sustained delivery of gemcitabine in the bladder. In phase 1 studies, TAR-200 was well-tolerated and showed preliminary efficacy in patients with muscle-invasive bladder cancer (MIBC) and intermediate-risk NMIBC with detectable levels of gemcitabine in urine 7 days after placement and no detectable gemcitabine in plasma. Moreover, in an animal model, gemcitabine metabolites were detected in all layers of bladder tissue 4 days after placement of TAR-200, demonstrating both sustained release and deep penetration of gemcitabine.

In the methods described herein, the formulations and intravesical systems described herein produce pharmacokinetic (PK) profiles that result in superior efficacy compared to conventional therapies, such as intravesical instillation. Such PK profiles result from continuous, sustained release of gemcitabine from the intravesical systems described herein. This is in stark contrast to intravesical instillation, wherein exposure of the bladder to gemcitabine lasts only until the individual voids the contents of the bladder. A flat urine exposure-efficacy relationship (comparable urine gemcitabine related component (GRC) amount, gemcitabine concentration, and gemcitabine amount) for complete response (CR) rate supports the choice of the 225 mg gemcitabine dose contained by the intravesical systems described herein.

In some embodiments, each intravesical system comprises about 256 mg of gemcitabine HCL. In some embodiments, the about 256 mg of gemcitabine HCL is equivalent to 225 mg free base. In some embodiments, each intravesical system comprises 256.3 mg of gemcitabine HCL. In some embodiments, this may be referred to as a 225 mg strength gemcitabine intravesical system.

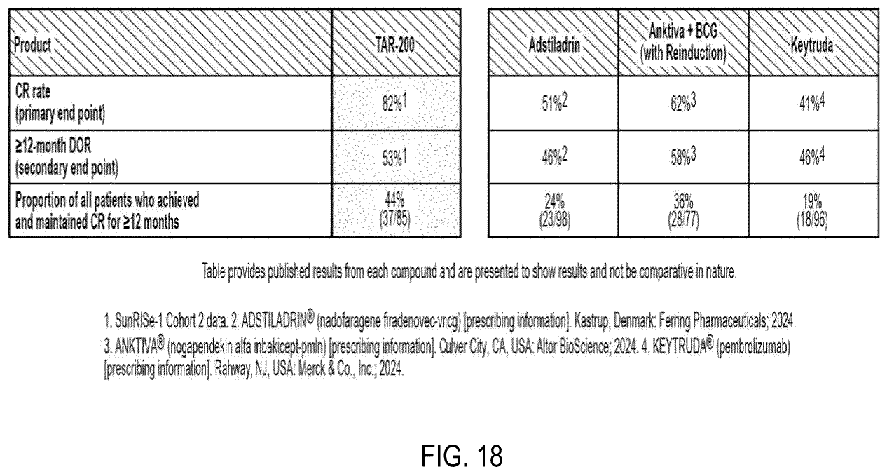

In a clinical study evaluating efficacy and safety of TAR-200 monotherapy in participants with Bacillus Calmette Guérin (BCG)-unresponsive High-Risk, non-muscle invasive bladder cancer (HR-NMIBC) with carcinoma in situ (CIS) with or without papillary tumors, TAR200 displayed unprecedented clinical outcomes compared to currently available therapies. The complete response (CR) rate for TAR-200 was 82%, significantly higher than nadofaragene firadenovec-vncg (51%), nogapendekin alfa inbakicept-pmln (62%), or pembrolizumab (41%). Further, TAR-200 induces a rapid onset of response, with patients experiencing a median time to onset of response of 2.8 months (FIG. 18).

### DEFINITIONS

Reference to “about” a value or parameter herein includes (and describes) variations that are directed to that value or parameter per se. For example, description referring to “about X” includes description of “X”. In another example, the term “about X-Y” used herein has the same meaning as “about X to about Y.”

In some embodiments, the term “about,” when used in association with a measurement or to modify a parameter or a value or a range of values, refers to variations of that measurement, parameter, value, or range of values of +/−10%, +/−9%, +/−8%, +/−7%, +/−6%, +/−5%, +/−4%, +/−3%, +/−2%, or +/−1%. For example, in some embodiments, “about X” includes and describes X+/−10%, +/−9%, +/−8%, +/−7%, +/−6%, +/−5%, +/−4%, +/−3%, +/−2%, or +/−1% of X. In some embodiments, the term “about” refers to variations of +/−5%, +/−4%, +/−3%, +/−2%, or +/−1%. In some embodiments, the term “about” refers to variations of +/−2% or +/−1%. In some embodiments, the term “about” refers to variations of +/−2%. In some embodiments, the term “about” refers to variations of +/−1%.

Reference to “at least” a value or parameter herein includes (and describes) variations that are directed to that value or parameter per se. For example, description referring to “at least X” includes description of “X”.

“Administration” or “administering” as used herein refers to providing a therapeutic agent to an individual. Administration can be intravenous, or intravesical, for example. In some examples, a particular amount of drug is administered to the individual, for example by inserting an intravesical system comprising the drug into the bladder of the individual, however only a portion of the drug is delivered (or released). In other embodiments, a particular amount of drug is administered to the individual, and the entire amount administered is delivered (or released) from the intravesical system.

“Continuous” or “continuously” as used herein refers to sustained or extended release of a therapeutic agent (such as gemcitabine) over an extended period of time. Continuous includes different release rates over a period of time. For example, a drug is continuously released over a period of seven days if the drug is released at a faster rate over the first three days and a slower rate over a period of the last four days.

“Delivery” as used herein refers to release of a drug. For example, a delivery period is a period during which an effective amount or clinically effective amount, of a drug, such as gemcitabine, is released from, for example, an intravesical system.

“Indwelling period” as used herein refers to a period in which an intravesical system is present in the bladder of an individual. For example, an intravesical system that is removed from a bladder three weeks after insertion has a three-week indwelling period.

“Individual” as used herein refers to a human.

“Treatment” or “treating” as used herein is an approach for obtaining beneficial or desired results including clinical results. For purposes of this invention, beneficial or desired clinical results include, but are not limited to, one or more of the following: alleviating one or more symptoms resulting from the disease, diminishing the extent of the disease, stabilizing the disease (e.g., preventing or delaying the worsening of the disease), suppressing or delaying the spread (e.g., metastasis) of the disease, preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. In some embodiments, “treatment” encompasses one or more clinical endpoints such as complete response rate, duration of complete response, and/or overall survival.

“Methods of treatment” as used herein is intended to be synonymous with “medical use.” For example, disclosure of “a method of treating BCG-unresponsive HR-NMIBC with CIS with or without papillary tumors” is equivalently to and includes “use of gemcitabine for treating BCG-unresponsive HR-NMIBC with CIS with or without papillary tumors in a patient.”

“Effective amount” as used herein refers to an amount of a compound or composition sufficient to treat a specified disorder, condition or disease such as to ameliorate, palliate, lessen, and/or delay one or more of its symptoms. In reference to cancers, an effective amount comprises an amount sufficient to cause a tumor to shrink and/or to decrease the growth rate of the tumor (such as to suppress tumor growth) and/or to suppress, or delay other unwanted cell proliferation and/or to treat or suppress tumor metastasis and/or to reduce (such as eradiate) preexisting tumor metastasis and/or to reduce incidence or burden of preexisting tumor metastasis and/or to suppress or delay tumor metastasis and/or to inhibit tumor cells and/or to induce an immune response against a tumor cell. An effective amount can be administered in one or more administrations, for example, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) suppress or delay occurrence and/or recurrence of tumor; and/or (vii) relieve to some extent one or more of the symptoms associated with the cancer.

“Monotherapy” as used herein means use of a treatment regimen, such as treatment comprising local administration of gemcitabine to the bladder, as the single active ingredient for treating bladder cancer, such as HR-NMIBC. In some embodiments, the individual receiving monotherapy receives one or more therapy to treat a disease other than bladder cancer or a side effect of the monotherapy.

“Pharmaceutically acceptable excipients” refer to solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

“Approved drug product” as used herein refers to a product that contains an active pharmaceutical ingredient that has been approved for marketing by a governmental authority, e.g., the Food and Drug Administration (FDA) or the similar authority in other countries.

As used herein, the singular forms “a,” “an,” and “the” are intended to include the plural forms as well, unless the context clearly indicates otherwise. For example, “a” or “an” means “at least one” or “one or more.”

It is also to be understood that the term “and/or” as used herein refers to and encompasses any and all possible combinations of one or more of the associated listed items.

It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

### Endpoint Definitions

“Complete Response (CR) Rate” as used herein is the percentage of patients achieving a complete response at any time post-treatment. Complete response was defined as at least one of the following: (1) negative cystoscopy and negative (including atypical) urine cytology, or (2) positive cystoscopy with biopsy-proven benign or low-grade NMIBC and negative (including atypical) urine cytology. For example, the 12-month CR rate is defined as the proportion of patients who have achieved a CR that lasts for at least 12 months.

“Duration of response (DOR)” is defined as the time from first CR to first evidence of recurrence or progression or death, whichever occurs first. DOR rate will be estimated using the Kaplan-Meier (KM) method. DOR rate is defined as the proportion of patients who have achieved a DOR that lasts for at least a set time period. For example, the 12-month DOR rate is defined as the proportion of patients who have achieved a DOR that lasts for at least 12 months.

“Overall survival (OS)” as used herein is defined as the time from the date of first dose of study treatment to death.

### Methods of Treatment

The various aspects and embodiments described in this section in the context of a method of treatment also apply to gemcitabine for use according to the methods described herein, and minitablets comprising gemcitabine for use according to the methods described herein, unless indicated otherwise. Similarly, the various aspects and embodiments described in this section in the context of a method of treatment also apply to gemcitabine for use with an intravesical system, according to the methods described herein, and gemcitabine for use according to the methods described herein, in combination with an intravesical system, and gemcitabine for use according to the methods described herein, wherein the gemcitabine is administered by an intravesical system.

Methods of treating bladder cancer in an individual are provided herein. In some embodiments, the methods comprise treating non-muscle invasive bladder cancer (NMIBC) in an individual. In some embodiments, the methods comprise treating High-Risk non-muscle invasive bladder cancer (HR-NMIBC) in an individual. In some embodiments, the methods comprise treating Bacillus Calmette-Guérin (BCG)-unresponsive, non-muscle invasive bladder cancer (NMIBC) with carcinoma in situ (CIS), with or without papillary tumors in an individual. In some embodiments, the methods comprise treating Bacillus Calmette Guérin (BCG)-unresponsive High-Risk, non-muscle invasive bladder cancer (HR-NMIBC) with carcinoma in situ (CIS) with or without papillary tumors in an individual. The methods described herein include administering to the bladder of the individual an intravesical system (e.g., TAR-200, also known as INLEXZO™), comprising about 225 mg of gemcitabine. In some embodiments, gemcitabine in the intravesical system (e.g., TAR-200) is in the form of a pharmaceutically acceptable salt. In some embodiments, gemcitabine is in the form of gemcitabine HCL. In some embodiments, the intravesical system comprises about 256 mg of gemcitabine HCL. In some embodiments, the intravesical system comprises about 225 mg of gemcitabine.

In some embodiments, the method comprises inserting an intravesical system (e.g., TAR-200) comprising about 256 mg of gemcitabine HCL into the bladder of the individual about once every three weeks for at least 24 weeks. In some embodiments, the method comprises inserting an intravesical system comprising about 256 mg of gemcitabine hydrochloride into the bladder once every 3 weeks up to 6 months (8 doses), followed by once every 12 weeks for up to 18 months, or until persistent or recurrent HR-NMIBC, disease progression, or unacceptable toxicity. In some embodiments, the method comprises inserting an intravesical system comprising about 256 mg of gemcitabine hydrochloride into the bladder once every 3 weeks up to 6 months (8 doses), followed by once every 12 weeks for up to 18 months, or until persistent or recurrent NMIBC, disease progression, or unacceptable toxicity.

In some embodiments, the method comprises inserting an intravesical system (e.g., TAR-200) comprising about 225 mg of gemcitabine into the bladder of the individual about once every three weeks for at least 24 weeks. In some embodiments, the method comprises inserting an intravesical system comprising about 225 mg of gemcitabine into the bladder once every 3 weeks up to 6 months (8 doses), followed by once every 12 weeks for up to 18 months (6 doses), or until persistent or recurrent NMIBC, disease progression, or unacceptable toxicity. In some embodiments, each intravesical system is removed after a 3-week indwelling period.

In some embodiments, the methods provided here reduce progression of patients with BCG-unresponsive NMIBC with CIS. In some embodiments, the median time between determination of persistent or recurrent CIS or T1 progression to muscle invasive disease is about 94 days.

Disclosed herein are methods of treating bladder cancer in an individual, the methods comprising administering to the individual an approved drug product comprising gemcitabine. The approved drug product comprising gemcitabine is described in the approved drug product label provided in Example 9 herein.

As used herein, “dose” refers to one cycle of inserting and removing an intravesical device. In some embodiments, if a dose is missed, it is administered as closely as possible to the original treatment schedule.

### Dosing Regimens

In some embodiments, the methods described herein comprise administering to the bladder of the individual a plurality of intravesical systems to treat bladder cancer. In some embodiments, about 256 mg of gemcitabine HCL is administered to the individual by each intravesical system. In some embodiments, 256.3 mg of gemcitabine HCL is administered to the individual by each intravesical system. In some embodiments, about 225 mg of gemcitabine is administered to the individual by each intravesical system. In some embodiments, the intravesical system comprises about 256 mg of gemcitabine HCL (equivalent to about 225 mg of gemcitabine free base).

In some aspects, provided herein is a method of treating bladder cancer comprising: inserting an intravesical system comprising about 225 mg of gemcitabine into the bladder of the individual about once every three weeks (Q3W) for a total of up to 6 months (i.e., ending in month 6 with the removal of an intravesical system) following starting treatment, wherein each intravesical system is removed from the bladder about three weeks after insertion into the bladder and wherein each intravesical system is removed prior to insertion of another intravesical system. In some embodiments, the intravesical system comprises about 256 mg of gemcitabine HCL. In some embodiments, the bladder cancer is NMIBC. In some embodiments, the bladder cancer is HR-NMIBC. In some embodiments, the bladder cancer is BCG-unresponsive HR-NMIBC with CIS with or without papillary tumors. In some embodiments, the bladder cancer is Bacillus Calmette Guérin (BCG)-unresponsive, non-muscle invasive bladder cancer (NMIBC) with carcinoma in situ (CIS) with or without papillary tumors.

In some aspects, provided herein is a method of treating BCG-unresponsive HR-NMIBC with CIS with or without papillary tumors in an individual comprising: inserting an intravesical system comprising about 225 mg of gemcitabine into the bladder of the individual about once every three weeks (Q3W) for a total of up to 6 months following starting treatment, followed by inserting an intravesical system comprising about 225 mg of gemcitabine into the bladder of the individual about once every 12 weeks (Q12W) for up to an additional 18 months after the first 6 months of treatment, wherein each intravesical system is removed from the bladder about three weeks after insertion into the bladder and wherein each intravesical system is removed prior to insertion of another intravesical system. In some embodiments, the method comprises inserting an intravesical system comprising about 225 mg of gemcitabine into the bladder of the individual about once every 12 weeks (Q12W) until about week 99 following starting treatment. In some embodiments, the intravesical system comprises about 256 mg gemcitabine HCL. In some embodiments, the bladder cancer is BCG-unresponsive NMIBC with CIS with or without papillary tumors.

In some aspects, provided herein is a method of treating BCG-unresponsive HR-NMIBC with CIS with or without papillary tumors in an individual comprising: inserting one intravesical system comprising about 225 mg of gemcitabine into the bladder of the individual per 21-day dosing cycle for about 8 consecutive cycles (doses), followed by inserting one intravesical system comprising about 225 mg of gemcitabine into the bladder of the individual per 21-day dosing cycle about once every 12 weeks (Q12W) until about 2 years following starting treatment, wherein each intravesical system is removed prior to insertion of another intravesical system. In some embodiments, the intravesical system comprises about 256 mg of gemcitabine HCL. In some embodiments, the bladder cancer is BCG-unresponsive NMIBC with CIS with or without papillary tumors.

In some embodiments, the method comprises administering to the bladder of the individual a plurality of intravesical systems during a first or induction phase. In some embodiments, administering the plurality of intravesical systems during a first phase comprises two or more three-week dosing cycles of gemcitabine comprising (i) inserting one of the plurality of intravesical systems into the bladders about once every three weeks (Q3W); and (ii) removing the intravesical system from the bladder about three weeks after insertion; wherein in each dosing cycle of gemcitabine steps (i) and (ii) of the dosing cycle of gemcitabine are repeated with a new intravesical system of the plurality of intravesical systems for up to about 24 weeks (6 months). In some embodiments, the first phase starts in week 0 with insertion of an intravesical system and ends with removal of an intravesical system on about week 24.

In some embodiments, the method comprises administering to the bladder of the individual a plurality of intravesical systems during a second or maintenance phase. In some embodiments, administering the plurality of intravesical systems during a second phase comprises two or more twelve-week dosing cycles of gemcitabine comprising (i) inserting one of the plurality of intravesical systems into the bladder about once every twelve weeks (Q12W); and (ii) removing the intravesical system from the bladder about three weeks after insertion; wherein in each dosing cycle of gemcitabine steps (i) and (ii) of the dosing cycle of gemcitabine are repeated with a new intravesical system of the plurality of intravesical systems for up to about 2 years. In some embodiments, the second phase starts on about week 24 with removal of an intravesical system and ends with the removal of an intravesical system on about week 99.

In some embodiments, the second phase begins on about week 24 and the first intravesical system in the second phase is inserted into the bladder in about week 36. In some embodiments, there is a period of time between the start of the second phase and the insertion of the first intravesical system of the second phase. In some embodiments that period of time is about 12 weeks.

In some embodiments, provided herein is a method of treating BCG-unresponsive HR-NMIBC with CIS with or without papillary tumors in an individual comprising: inserting an intravesical system comprising about 225 mg of gemcitabine into the bladder once every 3 weeks up to 24 weeks (6 months or 8 doses), followed by once every 12 weeks for up to 18 months. In some embodiments, provided herein is a method of treating BCG-unresponsive NMIBC with CIS with or without papillary tumors in an individual comprising: inserting an intravesical system comprising about 225 mg of gemcitabine into the bladder once every 3 weeks up to 6 months (8 doses), followed by once every 12 weeks for up to 18 months (6 doses). In some embodiments, the individual receives a total of 14 doses of gemcitabine over the 2 years of therapy. In some embodiments, the intravesical system comprises about 256 mg of gemcitabine HCl. In some embodiments, the bladder cancer is BCG-unresponsive NMIBC with CIS with or without papillary tumors.

In some embodiments, the first phase is an induction phase and the second phase is a maintenance phase.

In some embodiments, in the first phase, inserting the new intravesical system occurs within a week of removing a previous intravesical system. In some embodiments, inserting the new intravesical system occurs within three days of removing a previous intravesical system, such as within the same visit.

In some embodiments, the plurality of intravesical systems may be multiple copies of the same intravesical system. In other words, each intravesical system may have the same structure, shape, components, and the same amount of gemcitabine contained therein. In some embodiments, each intravesical system comprises TAR-200. In some embodiments, administering the plurality of intravesical systems during the first phase comprises the following steps:
i) inserting a first intravesical system into the bladder in week 0;
ii) removing the first intravesical system at about week 3;
iii) inserting a second intravesical system into the bladder at about week 3;
iv) removing the second intravesical system at about week 6;
v) inserting a third intravesical system into the bladder at about week 6;
vi) removing the third intravesical system at about week 9;
vii) inserting a fourth intravesical system into the bladder at about week 9;
viii) removing the fourth intravesical system at about week 12;
ix) inserting a fifth intravesical system into the bladder at about week 12;
x) removing the fifth intravesical system at about week 15;
xi) inserting a sixth intravesical system into the bladder at about week 15;
xii) removing the sixth intravesical system at about week 18;
xiii) inserting a seventh intravesical system into the bladder at about week 18;
xiv) removing the seventh intravesical system at about week 21;
XV) inserting an eighth intravesical system into the bladder at about week 21; and
xvi) removing the eighth intravesical system at about week 24, wherein each of the plurality of intravesical systems comprises about 225 mg gemcitabine (or about 256 mg gemcitabine HCL).

In any of the above embodiments, the previous intravesical system is removed before a subsequent intravesical system is inserted such that there is no more than one intravesical system in the bladder of the individual at any one time.

In some embodiments, the second phase comprises two or more dosing cycles of gemcitabine. In some embodiments, the second phase comprises up to six dosing cycles of gemcitabine.

In some embodiments, administering the plurality of intravesical systems during the second phase comprises the following steps:
i) inserting a ninth intravesical system into the bladder at about week 36;
ii) removing the ninth intravesical system at about week 39;
iii) inserting a tenth intravesical system into the bladder at about week 48;
iv) removing the tenth intravesical system at about week 51;
v) inserting an eleventh intravesical system into the bladder at about week 60;
vi) removing the eleventh intravesical system at about week 63;
vii) inserting a twelfth intravesical system into the bladder at about week 72;
viii) removing the twelfth intravesical system at about week 75;
ix) inserting a thirteenth intravesical system into the bladder at about week 84;
x) removing the thirteenth intravesical system at about week 87;
xi) inserting a fourteenth intravesical system into the bladder at about week 96;
and
xii) removing the fourteenth intravesical system at about week 99, wherein each of the plurality of intravesical systems comprises about 225 mg gemcitabine (or about 256 mg gemcitabine HCL).

In some embodiments, the method comprises the following steps:
i) inserting a first intravesical system into the bladder in week 0;
ii) removing the first intravesical system at about week 3;
iii) inserting a second intravesical system into the bladder at about week 3;
iv) removing the second intravesical system at about week 6;
v) inserting a third intravesical system into the bladder at about week 6;
vi) removing the third intravesical system at about week 9;
vii) inserting a fourth intravesical system into the bladder at about week 9;
viii) removing the fourth intravesical system at about week 12;
ix) inserting a fifth intravesical system into the bladder at about week 12;
x) removing the fifth intravesical system at about week 15;
xi) inserting a sixth intravesical system into the bladder at about week 15;
xii) removing the sixth intravesical system at about week 18;
xiii) inserting a seventh intravesical system into the bladder at about week 18;
xiv) removing the seventh intravesical system at about week 21; XV) inserting an eighth intravesical system into the bladder at about week 21;
xvi) removing the eighth intravesical system at about week 24;
xvii) inserting a ninth intravesical system into the bladder at about week 36;
xviii) removing the ninth intravesical system at about week 39;
xix) inserting a tenth intravesical system into the bladder at about week 48;
xx) removing the tenth intravesical system at about week 51;
xxi) inserting an eleventh intravesical system into the bladder at about week 60;
xxii) removing the eleventh intravesical system at about week 63;
xxiii) inserting a twelfth intravesical system into the bladder at about week 72;
xxiv) removing the twelfth intravesical system at about week 75;
XXv) inserting a thirteenth intravesical system into the bladder at about week 84;
xxvi) removing the thirteenth intravesical system at about week 87;
xxvii) inserting a fourteenth intravesical system into the bladder at about week 96; and
xxviii) removing the fourteenth intravesical system at about week 99, wherein each of the plurality of intravesical systems comprises about 225 mg gemcitabine (or about 256 mg gemcitabine HCL).

In any of the proceeding embodiments, the individuals treated with the methods described herein may have high-grade Ta or T1 urothelial carcinoma of the bladder. A urothelial carcinoma of the bladder is staged as “high-grade Ta” when the tumors are non-invasive papillary carcinomas. A urothelial carcinoma of the bladder is staged as “T1” when the tumors have grown into the connective tissue layer of the bladder wall, but it has not reached the layer of muscle in the bladder wall. In some embodiments, the individuals treated with the methods described herein have carcinoma in situ (CIS). In some embodiments, the individuals treated with the methods described herein do not have a urothelial carcinoma or histological variant at any site outside of the urinary bladder.

In some embodiments, the individuals treated with the methods described herein are ineligible for or had elected not to undergo radical cystectomy.

In some embodiments, the methods described herein improve an endpoint as described herein. In some embodiments, the methods described herein result in an increased complete response (CR) rate in a population of patients compared to the CR rate achieved by standard of care treatment. In some embodiments, the methods described herein result in an increased duration of response (DoR) in an individual and/or an increased DoR rate in a population of patients compared to the DoR and/or DoR rate achieved by standard of care treatment.

In some embodiments, the methods described herein comprise up to 14 total dosing cycles of gemcitabine over about 2 years of therapy.

In some embodiments, the methods described herein comprise a period between the first phase and second phase during which no intravesical system is present in the bladder. In some embodiments, this period is about 12 weeks. This period may be considered as part of the second phase, although no intravesical system is present in the bladder during this time.

In some embodiments, the methods described herein comprise a period between the first phase and second phase during which no gemcitabine is released into the bladder. In some embodiments, this period is about 12 weeks. This period may be considered as part of the second phase, although no gemcitabine is released into the bladder during this time.

In some embodiments, a delivery period is a period of time during which a therapeutically effective amount of gemcitabine is released from the intravesical system. In some embodiments, a therapeutically effective amount of gemcitabine is a urine concentration of about 0.1 μg/mL to about 3 μg/mL. In some embodiments, a therapeutically effective amount of gemcitabine is released from the intravesical system into the urine in the bladder during a delivery period that is less than the indwelling period. For example, the indwelling period may be about three weeks, although a therapeutically effective amount of gemcitabine may be released from the intravesical system over a delivery period of about one week, about two weeks, or any other time less than three weeks. The intravesical system may be configured to release the therapeutically effective amount of gemcitabine for a delivery period of at least about one week (e.g., 7 days). In some embodiments, the method comprises a delivery period of about 7 days.

In some embodiments, there is an effective concentration of gemcitabine in the urine of the individual during the gemcitabine delivery period. In some embodiments, the gemcitabine is continuously delivered to the bladder for about 7, 8, 9, 10, 11, 12, 13 or 14 days. In some embodiments, the gemcitabine is continuously delivered to the bladder for up to 21 days (e.g., for about 15, 16, 17, 18, 19, 20, or 21 days). In some embodiments, greater than 50% of the gemcitabine is delivered to the bladder within the first 7 days following insertion of the intravesical system.

The amount of gemcitabine released from the intravesical system can be estimated using in vitro or in vivo models. For example, the percent of gemcitabine released may be estimated using an in vitro release (IVR) model. The IVR model is determined by placing the intravesical system in water at 37 degrees C. and sampling the water over 21 days to quantify gemcitabine released from the intravesical system. In some embodiments, the IVR model is determined by: placing the intravesical system containing 225 mg gemcitabine (256 gemcitabine HCl) in 300 mL ultrapure water at 37 degrees C.; taking 1 mL samples at 1, 2, 3, 4, 7, 10, 14, 17, and 21 days after the system was placed in water; and analyzing the sample using an Acquity UPLC® (CSH C18 1.7-μm particle size, 50×2.1 mm i.d., Waters Corporation, Milford, MA) to quantify gemcitabine released from the intravesical system.

In a different example, the percent of gemcitabine released may be estimated using in vivo data, such as urine gemcitabine related component (GRC) amount-time profiles. From GRC amount-time profiles, the percent of GRC released is estimated using nonlinear mixed effect modeling. Such modeling may be based on parameters such as the proportion of gemcitabine available for release, the duration of zero- and first-order release, inter-individual variability, and/or residual variability. In some embodiments, the release of gemcitabine from the intravesical device is adequately described by a sequential zero- and first-order release.

In some embodiments, the majority of gemcitabine within the intravesical system is released in the first seven days of indwelling. In some embodiments, about 65% to about 85% of the gemcitabine in the intravesical system is released into urine in the individual's bladder over about the first 7 days after insertion into the bladder. In some embodiments, about 70% of the gemcitabine in the intravesical system is released into the urine over about the first 7 days after insertion into the bladder, wherein the percent of gemcitabine released is estimated using an in vitro release (IVR) model.

In some embodiments, about 76.9% of the gemcitabine in the intravesical system is released into the urine over about the first 7 days after insertion into the bladder, wherein the percent of gemcitabine released is estimated using in vivo data. In some embodiments, about 77% of the total gemcitabine dose in the intravesical system is released into the urine as gemcitabine and dFdU over about the first 7 days after insertion into the bladder, wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some embodiments, the intravesical system releases gemcitabine at a slower rate following the first seven days of indwelling. In some embodiments, about 85% to about 100% of the gemcitabine is release over about 21 days after insertion into the bladder. In some embodiments, about 90% of the gemcitabine in the intravesical system is released into the urine over about the first 21 days after insertion into the bladder, wherein the percent of gemcitabine released is estimated using an in vitro release model. In some embodiments, about 99% of the gemcitabine in the intravesical system is released into the urine over about the first 21 days after insertion into the bladder, wherein the percent of gemcitabine released is estimated using in vivo data. In some embodiments, about 99% of the total gemcitabine dose in the intravesical system is released into the urine as gemcitabine and dFdU over about the first 21 days after insertion into the bladder, wherein the percent of gemcitabine released is estimated using in vivo data. In some embodiments, the in vivo data comprise urine gemcitabine related component (GRC) amount-time profiles, wherein the percent of gemcitabine released is estimated using nonlinear mixed effect modeling.

In some embodiments, Gemcitabine and dFdU are excreted in urine throughout the indwelling period for INLEXZO™. In some embodiments, of the total gemcitabine dose, 77% is excreted by Day 7 and 99% is excreted by Day 21 in urine as gemcitabine and dFdU. In some embodiments, mean urinary excretion on Days 2 through 5 ranged from 21 to 32 mg per day excreted in the urine as gemcitabine and dFdU.

In the methods described herein, the intravesical systems described herein generate certain pharmacokinetic (PK) profiles that result in superior efficacy compared to conventional therapies, such as intravesical instillation. In some embodiments, individuals with complete response (CR) and with non-CR demonstrate comparable urine gemcitabine related components (GRC) amounts. Certain properties of the intravesical systems described herein, including the composition of the contents thereof, influence said PK profiles.

The intravesical systems described herein comprise gemcitabine minitablets and osmotic minitablets (for osmotic performance). In some embodiments, the gemcitabine minitablets comprise a total dose of about 256 mg gemcitabine HCL, polyethylene glycol 8000 (about 5 mg to about 12 mg, such as about 8 mg), povidone K30 (about 10 mg to about 20 mg, such as about 13.4 mg), and urea (about 35 mg to about 50 mg urea, such as about 42.6 mg). In some embodiments, each gemcitabine minitablet comprises about 19 mg to about 24 mg gemcitabine HCL, about 0.5 mg to about 1.5 mg Povidone 30, about 0.4 mg to about 1 mg polyethylene glycol (PEG) 8000, and about 2 mg to about 5 mg urea. Such values can also be expressed as percent weight, such that each gemcitabine minitablet comprises: about 72% to about 89% w/w gemcitabine HCL; about 1.6% to about 6.6% w/w Povidone 30; about 1.3% to about 4.4% w/w polyethylene glycol (PEG) 8000; and about 7% to about 20% w/w urea. In some embodiments, each gemcitabine minitablet comprises about 21.36 mg gemcitabine HCL, about 1.12 mg Povidone 30, about 0.67 mg polyethylene glycol (PEG) 8000, and about 3.55 mg urea. Such values can also be expressed as percent weight, such that each gemcitabine minitablet comprises: about 80% w/w gemcitabine HCL; about 4.2% w/w Povidone 30; about 2.5% w/w polyethylene glycol (PEG) 8000; and about 13.3% w/w urea.

In some embodiments, the osmotic minitablets comprise: urea (about 630 mg to about 670 mg), polyethylene oxide 600,000 (about 50 mg to about 90 mg), and colorant, such as FD&C Blue No. 1 (about 0.0020 mg to about 0.0060 mg). In some embodiments, each osmotic minitablet comprises about 15 mg to about 25 urea, about 1 mg to about 4 mg polyethylene oxide (PEO), and about 0.0005 mg to about 0.0020 mg colorant, such as FD&C Blue No. 1 colorant. Such values can also be expressed as percent weight, such that each osmotic minitablet comprises about 79% to about 96% w/w urea; about 4% to about 20% w/w polyethylene oxide (PEO); and about 0.0001% to about 0.0010% w/w colorant, such as FD&C Blue No. 1 colorant. In some embodiments, the osmotic (urea) minitablets comprise urea (about 648 mg), polyethylene oxide 600,000 (about 72 mg), and colorant, such as FD&C Blue No. 1 (about 0.0042 mg). In some embodiments, each osmotic minitablet comprises about 21.6 mg urea, about 2.40 mg polyethylene oxide (PEO), and about 0.0014 mg colorant, such as FD&C Blue No. 1 colorant. Such values can also be expressed as percent weight, such that each osmotic minitablet comprises about 90% w/w urea; about 10% w/w polyethylene oxide (PEO); and about 0.0006% w/w colorant, such as FD&C Blue No. 1 colorant.

In some embodiments, the PK profiles described herein are calculated based on data collected from use of the intravesical system in a monotherapy and use of the intravesical system in combination with cetrelimab. The PK profiles described herein can refer to amounts of the active metabolite (gemcitabine) or the inactive metabolite (dFdU), which is generated from deamination of gemcitabine. Also described by PK profiles are total excreted amount of gemcitabine related components (GRC), which is calculated as gemcitabine+dFdU. In some embodiments, such PK profiles are effective to treat bladder cancer. In some embodiments, such PK profiles are effective to treat NMIBC. In some embodiments, such PK profiles are effective to treat BCG-unresponsive, HR-NMIBC with CIS with or without papillary tumors. In some embodiments, such PK profiles are effective to treat BCG-unresponsive NMIBC with CIS with or without papillary tumors. In some embodiments, such PK profiles are effective to treat muscle invasive bladder cancer (MIBC).

In some embodiments, the PK profile has a mean maximum concentration (Cₘₐₓ) of gemcitabine of about 6 μg/mL to about 25 μg/mL, such as about 14.9 μg/mL. In some embodiments, the PK profile has a Cₘₐₓ of gemcitabine of about 6 μg/mL to about 25 μg/mL, such as about 12.5 μg/mL. In some embodiments, the PK profile has a Cₘₐₓ of gemcitabine of about 6 μg/mL to about 25 μg/mL, such as about 9.87 μg/mL. In some embodiments, the PK profile has a cycle day of maximum concentration (Tₘₐₓ) of gemcitabine of about 1 day to about 7 days, such as about 3 days. In some embodiments, the PK profile has a Tₘₐₓ of gemcitabine of about 1 day to about 7 days, such as about 2 days. In some embodiments, the PK profile has a mean cumulative excreted gemcitabine over 7 days of about 12% to about 62%, wherein the mean cumulative excreted gemcitabine is expressed as the percentage of the administered dose (amount of gemcitabine recovered/dose of gemcitabine free base). In some embodiments, the PK profile has a mean cumulative excreted gemcitabine over 7 days of about 37%. In some embodiments, the PK profile has a mean cumulative excreted gemcitabine over 7 days of about 31.3%. In some embodiments, such PK profiles are effective to treat bladder cancer. In some embodiments, such PK profiles are effective to treat NMIBC. In some embodiments, such PK profiles are effective to treat BCG-unresponsive, HR-NMIBC with CIS with or without papillary tumors. In some embodiments, such PK profiles are effective to treat BCG-unresponsive NMIBC with CIS with or without papillary tumors. In some embodiments, such PK profiles are effective to treat MIBC.

In some embodiments, the PK profile has a Cₘₐₓ of dFdU of about 1 μg/mL to about 20 μg/mL, such as about 10.5 μg/mL. In some embodiments, the PK profile has a Cₘₐₓ of dFdU of about 1 μg/mL to about 20 μg/mL, such as about 10.8 μg/mL. In some embodiments, the PK profile has a Cₘₐₓ of dFdU of about 1 μg/mL to about 20 μg/mL, such as about 15.6 μg/mL. In some embodiments, the PK profile has a Tₘₐₓ of dFdU about 1 day to about 7 days, such as about 4 days. In some embodiments, the PK profile has a Tₘₐₓ of dFdU about 1 day to about 7 days, such as about 3 days. In some embodiments, the PK profile has a Tₘₐₓ of dFdU about 1 day to about 7 days, such as about 2.5 days. In some embodiments, the PK profile has a mean cumulative excreted dFdU over 7 days of about 3% to about 53%, wherein the mean cumulative excreted gemcitabine is expressed as the percentage of the administered dose. In some embodiments, the PK profile has a mean cumulative excreted dFfU over 7 days of about 23.8%. In some embodiments, the PK profile has a mean cumulative excreted dFfU over 7 days of about 21.8%. In some embodiments, such PK profiles are effective to treat bladder cancer. In some embodiments, such PK profiles are effective to treat NMIBC. In some embodiments, such PK profiles are effective to treat BCG-unresponsive, HR-NMIBC with CIS with or without papillary tumors. In some embodiments, such PK profiles are effective to treat BCG-unresponsive NMIBC with CIS with or without papillary tumors. In some embodiments, such PK profiles are effective to treat MIBC.

In some embodiments, the PK profile has a mean cumulative excreted GRC over 7 days of about 50% to about 75%, wherein the mean cumulative excreted GRC is the percentage of the administered dose. In some embodiments, the PK profile has a mean cumulative excreted GRC over 7 days of about 61.3%. In some embodiments, the PK profile has a mean cumulative excreted GRC over 7 days of about 51.6%. In some embodiments, the PK profile has a mean cumulative excreted GRC of about 38% from Day 1 to Day 5 of treatment and about 9% from Day 8 to Day 12 of treatment. In some embodiments, such PK profiles are effective to treat bladder cancer. In some embodiments, such PK profiles are effective to treat NMIBC. In some embodiments, such PK profiles are effective to treat BCG-unresponsive, HR-NMIBC with CIS with or without papillary tumors. In some embodiments, such PK profiles are effective to treat BCG-unresponsive NMIBC with CIS with or without papillary tumors. In some embodiments, such PK profiles are effective to treat MIBC.

In some embodiments, the PK profile has a mean excreted GRC of about 2 mg to about 21 mg on day 1 after insertion in the bladder, about 17 mg to about 37 mg on day 2 after insertion in the bladder, about 20 mg to about 40 mg on day 3 after insertion into the bladder, about 12 mg to about 38 mg on day 4 after insertion into the bladder, about 5 mg to about 35 mg on day 5 after insertion into the bladder, about 2 mg to about 27 mg on day 6 after insertion into the bladder, and/or about 1 mg to about 20 mg on day 7 after insertion into the bladder. In some embodiments, the PK profile has a maximum mean amount of about 31.5 mg GRC (gemcitabine and dFdU) in urine on Day 3. In other words, in some embodiments, the highest urinary excretion occurs on Day 3 with a mean of 31.5 mg gemcitabine excreted in the urine as gemcitabine and dFdU. In some embodiments, each intravesical system produces a mean urinary excretion on Days 2 through 5 of 21 mg to 32 mg per day as gemcitabine and dFdU. In some embodiments, such PK profiles are effective to treat bladder cancer. In some embodiments, such PK profiles are effective to treat NMIBC. In some embodiments, such PK profiles are effective to treat BCG-unresponsive, HR-NMIBC with CIS with or without papillary tumors. In some embodiments, such PK profiles are effective to treat BCG-unresponsive NMIBC with CIS with or without papillary tumors. In some embodiments, such PK profiles are effective to treat MIBC.

In some embodiments, each intravesical system releases about 2% to about 20% of the gemcitabine on each of days 1-7 after the intravesical system is inserted into the bladder of the individual. In some embodiments, each intravesical system produces an average urine concentration of about 2 μg/mL to about 15 μg/mL on about days 1-7 after insertion into the bladder of the individual.

The local administration of gemcitabine by the intravesical systems described herein results in reduced side effects compared to systemic chemotherapy. For example, in some embodiments, the intravesical systems described herein produce a systemic exposure of gemcitabine in the individual of less than about 0.1 μg/mL.

In some embodiments, as used herein, “about three weeks” refers to a dosing window of ±3 days or ±7 days. In some embodiments, as used herein, “about twelve weeks” refers to a dosing window of ±1 week. Week 0, Day 1 is the day of the first dose of the first treatment administered.

### Patient Populations

The present invention, in some aspects, provides a method of treating bladder cancer in an individual. In some aspects, provided herein are methods of treating NMIBC in an individual. In some aspects, provided herein are methods of treating BCG-unresponsive NMIBC with CIS with or without papillary tumors. In some aspects, provided herein are methods of treating HR-NMIBC in an individual. In some aspects, provided herein are methods of treating BCG-unresponsive HR-NMIBC with CIS with or without papillary tumors in an individual. In some aspects, provided herein are methods of treating BCG-unresponsive NMIBC with CIS with or without papillary tumors. In some embodiments, the individual has a histologically variant subtype of a urothelial carcinoma of the lower tract, such as papillary, micropapillary, or carcinoma in situ. In some embodiments, the carcinoma in situ is lobular carcinoma in situ (LCIS) or ductal carcinoma in situ. In some embodiments, the individual is at risk of disease progression.

In some embodiments, the individual is unfit or not eligible for a cystectomy or has refused a cystectomy. In some embodiments, the individual is ineligible for radical cystectomy under the National Comprehensive Cancer Network (NCCN) guidelines. For example, the individual is unfit for curative therapy due to frailty. Prior to the present methods, such individuals typically received palliative radiation without chemotherapy (3.5 Gy/fraction-10 treatments; or 7 Gy/fraction-7 treatments; TURBT; or no treatment). In some embodiments, the individual cannot tolerate radical cystectomy based upon the American Society of Anesthesiology (ASA) guidelines. For example, the individual who cannot tolerate radial cystectomy may be deemed medically unfit for surgery requiring general or epidural anesthesia.

In other embodiments, the individual may not be suitable for radical cystectomy due to a lack of post-operative care infrastructure (e.g., as determined by the Comprehensive Geriatric Assessment provided by the American Society of Anesthesiologists). In some embodiments, an individual is not suitable for radical cystectomy due to frailty (e.g., as determined by the Comprehensive Geriatric Assessment provided by the American Society of Anesthesiologists). Under these guidelines, an individual is deemed frail if he or she shows abnormal independent activities of daily living, severe malnutrition, cognitive impairment, or comorbidities cumulative illness rating scale for geriatrics (CISR-G) grades 3-4. Furthermore, under current guidelines, subjects must be deemed unfit for radical cystectomy (RC) due to comorbid conditions with a risk of mortality ≥5% as estimated by the American College of Surgeons risk calculator using the current procedure terminology code 51595 or 51596 for cystectomy.

In some embodiments, the individual is eligible for a radical cystectomy but elects not to undergo the radical cystectomy due to quality-of-life considerations. In some embodiments, the quality-of-life impacts of radical cystectomy include, but are not limited to, mortality, incontinence, decreased sexual function, subfertility or infertility, and decreased bowel function. In some embodiments, more than about 40% of patients in a population of patients refuse radical cystectomy to preserve their bladders. In some embodiments, between about 45% to about 55% of patients in a population of patients refuse radical cystectomy to preserve their bladders. In some embodiments, at least about 45% of patients in a population of patients refuse radical cystectomy to preserve their bladders. In some embodiments, about 51% of patients in a population of patients refuse radical cystectomy to preserve their bladders. In some embodiments, more than about 30% of patients in a population of patients refuse radical cystectomy because of morbidity and mortality concerns. In some embodiments, between about 30% to about 45% of patients in a population of patients refuse radical cystectomy because of morbidity and mortality concerns. In some embodiments, at least about 35% of patients in a population of patients refuse radical cystectomy because of morbidity and mortality concerns. In some embodiments, about 37% of patients in a population of patients refuse radical cystectomy because of morbidity and mortality concerns.

In some embodiments, the individual is assessed based on an ECOG Performance Status Scale. In some embodiments, the ECOG performance scale measures an individual's level of functioning in terms of their ability to care for themselves, daily activity, and physical ability. In some embodiments, an individual with an ECOG performance status of 0 is fully active, and able to carry on all pre-disease performance without restriction. In some embodiments, an individual with an ECOG performance status of 1 is restricted in physically strenuous activity but ambulatory and able to carry out work of a light or sedentary nature. In some embodiments, an individual with an ECOG performance status of 2 is ambulatory and capable of all selfcare but unable to carry out any work activities. In some embodiments, the individual has an ECOG performance status of 0-2. In some embodiments, the individual has an ECOG performance status of 0. In some embodiments, the individual has an ECOG performance status of 1. In some embodiments, the individual has an ECOG performance status of 2.

In some aspects, the individual has non-muscle invasive bladder cancer (NMIBC). In some embodiments, NMIBC can be risk stratified into low, intermediate and high-risk groups depending on the probability of recurrence and progression to muscle-invasive disease. In some embodiments, the individual has high-risk NMIBC (HR-NMIBC), such as carcinoma in situ (CIS). In some embodiments, the individual has high-risk NMIBC (CIS with or without papillary disease) unresponsive to prior intravesical Bacillus Calmette-Guerin (BCG) therapy. In some embodiments, the individual is either ineligible for or has elected not to undergo radical cystectomy (RC). In some embodiments, the individual is diagnosed with high-risk, non-muscle invasive urothelial carcinoma in situ (CIS; Tis), with or without papillary disease (T1, high-grade Ta) within 12 months of completion of adequate BCG therapy. In some embodiments, the individual has BCG unresponsive NMIBC CIS, defined as persistent or recurrent CIS alone or with Ta/T1 disease within 12 months of adequate BCG therapy. In some embodiments, the individual has BCG-unresponsive high-risk NMIBC after treatment with adequate BCG therapy defined as a minimum of 5 of 6 doses of an induction course (adequate induction) plus 2 of 3 doses of a maintenance course, or 2 of 6 doses of a second induction course. In some embodiments, the individual is eligible for a radical cystectomy. In some embodiments, prior to treatment the individual has undergone transurethral resection of bladder tumor (TURBT).

In some embodiments, the NMIBC is staged using the tumor, node, metastasis (TNM) staging system. In some embodiments, Ta stage bladder cancer is defined as a non-invasive papillary carcinoma. In some embodiments, Ta stage bladder cancer has grown toward the hollow center of the bladder but has not grown into the connective tissue or muscle of the bladder wall. In some embodiments, Ta stage bladder cancer is further delineated into either low-grade (LG) or high-grade (HG) subtypes, with LG referring to a slow growing, less aggressive form of the disease and HG referring to a rapidly growing, more aggressive form of the disease. In some embodiments, CIS is defined as a flat lesion comprising of cytologically malignant cells which may involve either full or partial thickness of the urothelium. In some embodiments, the NMIBC is identified by clinical staging (cTa, cT1) based on endoscopic surgery (biopsy or TURBT).

In some embodiments, the individual has undergone prior BCG therapy. In some embodiments, the prior BCG treatment comprises intravesical instillations of hundreds of millions of Mycobacterium bovis Bacillus Calmette-Calmette-Guérin bacilli applied weekly over the course of a six week induction treatment. In some embodiments, the bladder cancer is resected following the six-week BCG induction treatment. In some embodiments, the prior BCG therapy further comprises a maintenance treatment consisting of six-week periods of intravesical BCG instillation every three months for one to three years.

In some embodiments, the individual has undergone between 3 and 6 courses of BCG induction treatment. In some embodiments, the individual has undergone at least 1 to 2, 2 to 3, 3 to 4, 4 to 5, or 5 to 6 prior courses of BCG induction treatment. In some embodiments, the individual has undergone 5 courses of BCG induction treatment during the prior BCG therapy. In some embodiments, the individual has undergone at least 7, 8, 9, 10, 11, 12, 13, 14, 15, or 16 prior courses of BCG therapy. In some embodiments, adequate BCG therapy is defined as a minimum of 5 of 6 full doses of an induction course plus 2 of 3 doses of a maintenance course, or at least 2 of 6 doses of a second induction course. In some embodiments, individuals receiving BCG therapy receive screening endoscopic surgeries (biopsies or TURBTs) about every 3 months to monitor for disease progression.

In some embodiments, BCG-unresponsive HR-NMIBC is defined as persistent disease despite adequate BCG therapy, disease recurrence after initially achieving a tumor-free state after adequate BCG, or T1 tumors following a single induction course of BCG. In some embodiments, an individual becomes aware of BCG-unresponsive HR-NMIBC after experiencing painful urination (dysuria). In some embodiments, an individual becomes aware of BCG-unresponsive HR-NMIBC after experiencing blood in the urine without pain (asymptomatic microhematuria). In some embodiments, BCG unresponsive disease is defined as recurrent high-grade Ta or any T1 disease within 6 months of completion of adequate BCG therapy, or T1 high-grade disease at the first disease assessment following an induction BCG course.

In some embodiments, BCG-unresponsive NMIBC is defined as persistent disease despite adequate BCG therapy, disease recurrence after initially achieving a tumor-free state after adequate BCG, or T1 tumors following a single induction course of BCG. In some embodiments, an individual becomes aware of BCG-unresponsive HR-NMIBC after experiencing painful urination (dysuria). In some embodiments, an individual becomes aware of BCG-unresponsive NMIBC after experiencing blood in the urine without pain (asymptomatic microhematuria). In some embodiments, BCG unresponsive disease is defined as recurrent high-grade Ta or any T1 disease within 6 months of completion of adequate BCG therapy, or T1 high-grade disease at the first disease assessment following an induction BCG course.

In some embodiments, the bladder cancer is resected prior to administration of the gemcitabine. In some embodiments, the individual undergoes a TURBT prior to administration of the gemcitabine to the bladder (i.e., before week 0 of treatment). In some embodiments, gemcitabine is administered within seven days following a TURBT. In some embodiments, the tumor is maximally resected prior to administration of the gemcitabine such that no visible tumor is present. In some embodiments, the tumor is non-maximally resected prior to administration of the gemcitabine. In some embodiments, the tumor is non-maximally resected prior to administration of the gemcitabine such that residual tumor is present. In some embodiments, the patient is TO after TURBT. In some embodiments, the individual undergoes TURBT at week 24 and/or week 48 of treatment with gemcitabine.

In some embodiments, the individual has undergone TURBT and has residual tumor at the site of resection. In some embodiments, the individual has stage Ta, or Tis cancer following TURBT.

In some embodiments, the individual does not have lymph node involvement (N+) and/or metastases (M+) per Blinded Independent Central Review of CT/MR Urography. In some embodiments, the individual does not have active malignancies other than the disease being treated under study. In some embodiments, active malignancies are defined as malignancies progressing or requiring treatment change in the last 24 months.

In some embodiments, the individual is renally impaired and/or hepatically impaired. In some embodiments, the individual is renally impaired. In some embodiments, renal impairment is measured using a creatine clearance rate calculated using the Cockcroft-Gault formula. In some embodiments, the individual is hepatically impaired. In some embodiments, hepatic function is measured with total bilirubin, alanine aminotransferase (ALT), or aspartate aminotransferase (AST) levels.

In some embodiments, the individual is elderly. In some embodiments, the individual is at least 65 years old. In some embodiments, the individual is between about 65 to about 70, about 70 to about 75, about 75 to about 80, about 80 to about 85, or about 85 to about 90 years old. In some embodiments, the individual is more than 65 years old. In some embodiments, the individual is frail.

In some embodiments, there are at least 20 individuals in the population of patients. In some embodiments, there are between about 20 and about 30, about 30 and about 40, about 40 and about 50, about 50 and about 60, about 60 and about 70, about 70 and about 80, about 80 and about 90, or about 90 and about 100 individuals in the population of patients. In some embodiments, there are 85 individuals in the population of patients. In some embodiments, there are 83 individuals in the population of patients. In some embodiments, there are 71 individuals in the population of patients.

### Endpoints

In some embodiments, provided herein is a method of treating bladder cancer in an individual comprising inserting an intravesical system comprising about 256 mg of gemcitabine HCL into the bladder of the individual once every three weeks for at least 24 weeks, wherein the individual experiences a complete response (CR). In some embodiments, the complete response is defined as negative cystoscopy and negative (including atypical) urine cytology, or positive cystoscopy with biopsy-proven benign or low-grade NMIBC and negative (including atypical) urine cytology. In some embodiments, the complete response rate is determined from the treated analysis set. In some embodiments, the treated analysis set includes the evaluable analysis set and individuals who discontinued from the study. In some embodiments, the evaluable analysis set is defined as all treated participants who had active disease at baseline and adequate disease assessment post-baseline, or who had progressed, died, or was withdrawn from treatment due to recurrence of high-risk disease or progressive disease. In some embodiments, the complete response rate is determined from the evaluable analysis set. In some embodiments, the individual has a durable complete response. In some embodiments the CR rate is the percentage of patients achieving a complete response at any time post-treatment. In some embodiments, individuals who remain free of recurrence of high grade disease 12 months after treatment have achieved a durable complete response. In some embodiments, the bladder cancer is NMIBC. In some embodiments, the bladder cancer is HR-NMIBC. In some embodiments, the bladder cancer is BCG-unresponsive HR-NMIBC with CIS with or without papillary tumors. In some embodiments, the bladder cancer is BCG-unresponsive NMIBC with CIS with or without papillary tumors.

In some embodiments, provided herein is a method of treating bladder cancer in an individual comprising inserting an intravesical system comprising about 256 mg of gemcitabine HCL into the bladder of the individual once every three weeks for at least 24 weeks, wherein such treatment results in a complete response (CR) rate of at least 50% in a population of patients who have received such treatment. In some embodiments, the complete response rate in a population of patients who have received such treatment is between about 50% and 60%, about 60% and 70%, about 70% and 80%, about 80% and 90%, about 90% and 100%. In some embodiments, the CR in a population of patients who have received such treatment is greater than about 51%. In some embodiments, the CR in a population of patients who have received such therapy is about 51% to about 87%. In some embodiments, the CR in a population of patients who have received such therapy is about 70% to about 90%. In some embodiments, the CR in a population of patients who have received such therapy is about 72%, about 73%, about 74%, about 75%, about 76%, about 77%, or about 78%. In some embodiments, the CR is about 70% to about 80%. In some embodiments, the 95% confidence interval of a complete response rate in the population of patients is between about 70% to about 95%, such as about 74% to about 91%. In some embodiments, the 95% confidence interval of a complete response rate in the population of patients is between about 70% to about 95%, such as about 72% to about 90%. In some embodiments, the 95% confidence interval of a complete response rate in the population of patients is between about 70% to about 90%. In some embodiments, the complete response rate in a population of patients who have received such treatment is about 84%. In some embodiments, the complete response rate in a population of patients who have received such treatment is about 82%. In some embodiments, the complete response rate in a population of patients who have received such treatment is about 82.4%, wherein the complete response rate is centrally-assessed (i.e., assessed by an independent central review). In some embodiments, the complete response rate in a population of patients who have received such treatment is about 83.5%, wherein the complete response rate is investigator-assessed (i.e., assessed by the local investigator). In some embodiments, the individual has a complete response within 3 months following the start of administration of gemcitabine. In some embodiments, the bladder cancer is HR-NMIBC. In some embodiments, the bladder cancer is BCG-unresponsive HR-NMIBC with CIS with or without papillary tumors. In some embodiments, the bladder cancer is BCG-unresponsive NMIBC with CIS with or without papillary tumors.

In some embodiments, the 12-month CR rate is defined as the proportion of patients who have achieved a CR that lasts for at least 12 months. In some embodiments, the 12-month CR rate in the population of patients of at least about 40%. In some embodiments, the 12-month CR rate in the population of patients is between about 40% to about 70%. In some embodiments, the 95% confidence interval of a 12-month CR rate in the population of patients is between about 35% to about 65%, such as about 36% to about 62%. In some embodiments, the 12-month CR rate in the population of patients is between about 40% to about 50%, about 50% to about 60%, about 60% to about 70%, about 70% to about 80%. In some embodiments, the 12-month CR rate is calculated from all treated patients in the population of patients. In some embodiments, the 12-month CR rate in the population of patients is about 50%. In some embodiments, the 12-month CR rate in the population of patients is about 46%, such as 45.9%.

In some embodiments, the proportion of all patients who achieve and maintain CR for ≥12 months is between about 35% to about 55%, such as about 44%. Said differently, in some embodiments, the proportion of all patients who maintain at least 12 months of DOR from the onset of CR is about 35% to about 55%, such as about 44%.

Similarly, 3- and 6-month CR rates can be defined as the proportion of patients who have achieved a CR that lasts for at least 3 and 6 months, respectively. In some embodiments, the 3-month CR rate in the population of patients is about 65% to about 90%, such as about 78.8%. In some embodiments, the 6-month CR rate in the population of patients is about 47% to about 70%, such as about 58.8%.

The methods described herein can provide a rapid onset of response. In some embodiments, a median time to onset of response is defined as the time from the start of treatment until a CR is achieved. In some embodiments, the methods of treatment described herein result in a median time to onset of response of about 2 months to about 9 months, such as about 2.8 months. In some embodiments, about 90% to about 99% (such as about 94.4%) of responders achieve CR by a first disease evaluation. In some embodiments, about 95.7% of responders achieve CR by a first disease evaluation. The first disease evaluation can occur at about 3 months following the start of treatment. In some embodiments, the CR rate at 6 months is about 60% to about 70%, such as about 66.2%. In some embodiments, the CR rate is calculated using a Kaplan Meier (KM) estimation. In some embodiments, the KM-estimated CR at 12 months is about 40% to about 65%, such as about 52.4%. In some embodiments, the KM-estimated CR at 12 months is about 56.2%. In some embodiments, the KM-estimated CR at 18 months is about 30% to about 65%, such as about 48.3%. In some embodiments, the KM-estimated CR at 18 months is about 52.4%. In some embodiments, the KM-estimated CR at 24 months is about 30% to about 65%, such as about 48.3%. In some embodiments, the KM-estimated CR at 24 months is about 44.7%.

In some embodiments, provided herein is a method of treating bladder cancer in an individual comprising inserting an intravesical system comprising about 256 mg of gemcitabine HCL into the bladder of the individual once every three weeks for at least 24 weeks, wherein such treatment results in a complete response rate that is at least 1.5 fold the complete response rate achieved by the standard of care. In some embodiments, such treatment results in a complete response rate that is between about 1.5 and 1.7, 1.7 and 1.9, 1.9 and 2.1, or 2.1 and 2.3 fold the complete response rate achieved by the standard of care. In some embodiments, such treatment results in a complete response rate that is between about 1.5 and 1.7, 1.7 and 1.9, 1.9 and 2.1, or 2.1 and 2.3 fold the complete response rate achieved by valrubicin. In some embodiments, such treatment results in a complete response rate that is between about 1.5 and 1.7, 1.7 and 1.9, 1.9 and 2.1, or 2.1 and 2.3 fold the complete response rate achieved by a checkpoint inhibitor therapy. In some embodiments, the bladder cancer is HR-NMIBC. In some embodiments, the bladder cancer is BCG-unresponsive HR-NMIBC with CIS with or without papillary tumors. In some embodiments, the bladder cancer is BCG-unresponsive NMIBC with CIS with or without papillary tumors.

In some embodiments, such treatment results in a median duration of response (DOR) that is between about 9 and 30 months. In some embodiments, the median DOR is at least 9 months, at least 10 months, at least 11 months, at least 12 months, at least 13 months, or at least 14 months. In some embodiments, the median DOR is between about 10 and 11 months. In some embodiments, the median DOR is between about 25 and about 27 months. In some embodiments, the median DOR is about 11 months. In some embodiments, the median DOR is about 26 months. In some embodiments, the median DOR is about 25.8 months.

In some embodiments, such treatment results in a 12-month DOR rate of between about 43% and about 67%, such as about 55.7%. In some embodiments, the 12-month DOR rate is about 52.9%. Said differently, in some embodiments, about 52.9% of responses lasted for ≥1 year. In some embodiments, the 12-month DOR rate is about 51%. Said differently, in some embodiments, about 51% of responses lasted for >1 year. In some embodiments, the 3-month DOR rate is between about 90% and about 99%, such as about 93.8%. In some embodiments, the 6-month DOR rate is between about 70% and about 80%, such as about 74.2%. In some embodiments, the 9-month DOR rate is between about 55% and about 65%, such as about 59.2%. In some embodiments, the 15-month DOR rate is between about 30% and about 40%, such as about 35.1%. In some embodiments, the 18-month DOR rate is between about 25% and about 35%, such as about 31.4%. In some embodiments, the 24-month DOR rate is between about 28% and about 54%, such as about 41.6%. Said differently, in some embodiments, about 42% of responses lasted for >2 years. In some embodiments, the 24-month cystectomy-free rate is between about 61% and about 83%, such as about 73.7%.

In some embodiments, a CR is ongoing after between about 9 and about 18 months of follow-up. In some embodiments, a CR is ongoing after at least 9 months, at least 10 months, at least 11 months, at least 12 months, at least 13 months, or at least 14 months of follow-up. In some embodiments, a CR is ongoing after a median of 10.6 months of follow-up. In some embodiments, the median DOR in the population of patients is not reached within 10.6 months. In some embodiments, a CR is ongoing after a median of 48 weeks of follow-up. In some embodiments, the median DOR in the population of patients is not reached within about 48 weeks. In some embodiments, the DOR measurement is calculated from patients who have responded to treatment. In some embodiments, DOR is defined as the date of first CR achieved to the date of first evidence of recurrence or progression or death, using cystoscopy, centrally read bladder biopsy and urine cytology, and imaging, if available.

In some embodiments, the methods described herein results in an overall survival rate at 6 months of about 91% to 99.9%, such as about 98.7%. In some embodiments, the overall survival rate at 12 months is about 86% to about 98%, such as about 94.7%.

In some embodiments, a complete response is observed during a scheduled cystoscopy and cytology assessment during treatment. In some embodiments, the results of the treatments described herein are compared to a standard of care treatment. In some embodiments, the standard of care treatment comprises radical cystectomy. In some embodiments, the standard of care treatment comprises a systemic or intravesical chemotherapy. In some embodiments, the standard of care treatment comprises mitomycin C therapy. In some embodiments, the standard of care treatment comprises Valrubicin. In some embodiments, the standard of care treatment comprises pembrolizumab. In some embodiments, the standard of care treatment comprises paclitaxel. In some embodiments, the standard of care treatment comprises an immune checkpoint therapy such as pembrolizumab. In some embodiments, the standard of care treatment includes an adjuvant chemotherapy such as mitomycin C, epirubicin, or doxorubicin. In some embodiments, the standard of care treatment comprises a systemic or intravesical combination chemotherapy. In some embodiments, the combination intravesical chemotherapy is composed of gemcitabine and docetaxel.

In some embodiments, following any of the treatments described herein, the individual has a biopsy-proven benign or low-grade NMIBC. In some embodiments, the biopsy-proven benign or low-grade NMIBC comprises a low-grade Ta bladder cancer. In some embodiments, the low-grade NMIBC is a non-invasive papillary (Ta) tumor.

In some embodiments, the methods provided herein result in bladder sparing in an individual with bladder cancer. In some embodiments, the bladder sparing methods provided herein result in significant improvements on quality of life for treated individuals. In some embodiments, a quality-of-life score is calculated for treated individuals based on results from the European Organization for Research and Treatment of Cancer-Quality of Life Questionnaire-Core (EORTC-QLQ-C30) or European Organization for Research and Treatment of Cancer-Non-Muscle Invasive Bladder Cancer Questionnaire (EORTC-QLQ-NMIBC24). In some embodiments, the quality-of-life score of the individual is maintained or increases following treatment by the methods provided herein. In some embodiments, quality of life improvements include avoiding the risks of mortality and morbidity associated with radical cystectomy, as well as maintaining continence, sexual function, fertility, and bowel function. In some embodiments, the bladder cancer is HR-NMIBC. In some embodiments, the bladder cancer is BCG-unresponsive HR-NMIBC with CIS with or without papillary tumors. In some embodiments, the bladder cancer is BCG-unresponsive NMIBC with CIS with or without papillary tumors.

The EORTC-QLQ-C30 is a core questionnaire for evaluating the quality of life of participants participating in cancer clinical studies. In some embodiments, the EORTC-QLQ-C30 is a 30-item questionnaire with 9 multi-item subscales and 6 single items. In some embodiments, the EORTC-QLQ-C30 incorporates 5 functional scales (physical, role, cognitive, emotional, and social functioning), 3 symptom scales (fatigue, pain, and nausea or vomiting), and a global health status or quality of life scale. In some embodiments, the methods provided herein result in a mean global health status score of about 55 to about 95, such as about 75. In some embodiments, the methods provided herein result in a mean physical functioning score of about 66 to about 99, such as about 86. In some embodiments, the single items assess dyspnea, appetite loss, sleep disturbance, constipation, diarrhea, and perceived financial impact of disease and treatment. Ratings for each item range from 1 (not at all) to 4 (very much). In some embodiments, the EORTC-QLQ-C30 is designed to be used across cancer populations and takes about 11 minutes to complete.

The EORTC-QLQ-NMIBC24 is a 24-item questionnaire for evaluating the quality of life of participants with superficial (non-muscle invasive) bladder cancer. In some embodiments, the questionnaire is designed to supplement the QLQ-C30 aand incorporates 6 multi-item scales and 5 single items. In some embodiments, ratings for each item in the EORTC-QLQ-NMIBC24 range from 1 (not at all) to 4 (very much). In some embodiments, the scales cover urinary symptoms, malaise, worries about the future, bloating and flatulence, sexual function, and male sexual problems. In some embodiments, the single items assess intravesical treatment issues, sexual intimacy, sexual enjoyment, risk of contaminating partner, and female sexual problems. In some embodiments, the EORTC-QLQ-NMIBC24 takes about 8 minutes to complete.

### Intravesical Systems

The various aspects and embodiments described in this section in the context of a method of treatment comprising administering gemcitabine using an intravesical system also apply to gemcitabine for use according to the methods described herein, and minitablets comprising gemcitabine for use according to the methods described herein. In particular, the aspects and embodiments described in this section also relate to gemcitabine for use with an intravesical system for use in the methods provided herein, wherein the method comprises continuous delivery of gemcitabine to the bladder by an intravesical system comprising the intravesical system, and to gemcitabine for use according to the methods described herein, wherein the gemcitabine is administered by an intravesical system. For example, the intravesical systems described in this section can be used for the treatment of bladder cancer in an individual. In some embodiments, the bladder cancer is HR-NMIBC. In some embodiments, the bladder cancer is BCG-unresponsive HR-NMIBC with CIS with or without papillary tumors. In some embodiments, the bladder cancer is BCG-unresponsive NMIBC with CIS with or without papillary tumors.

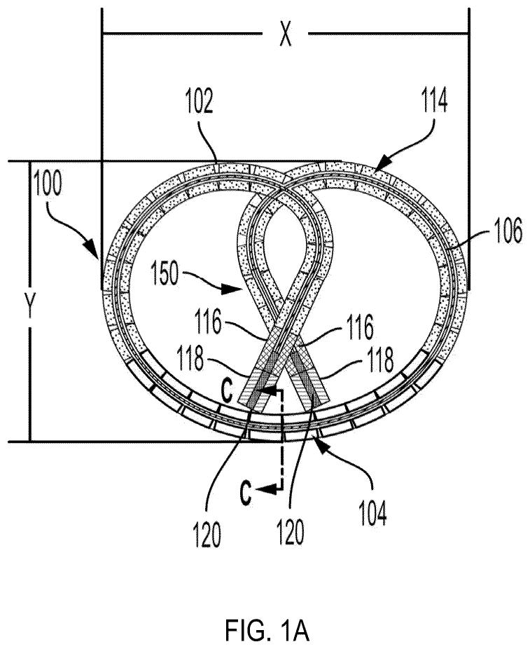

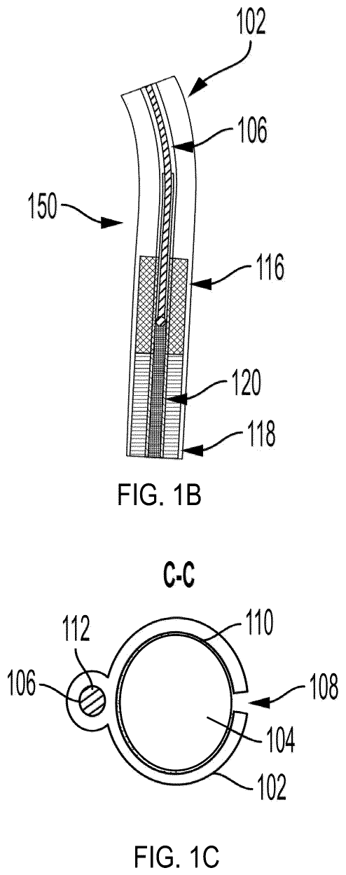

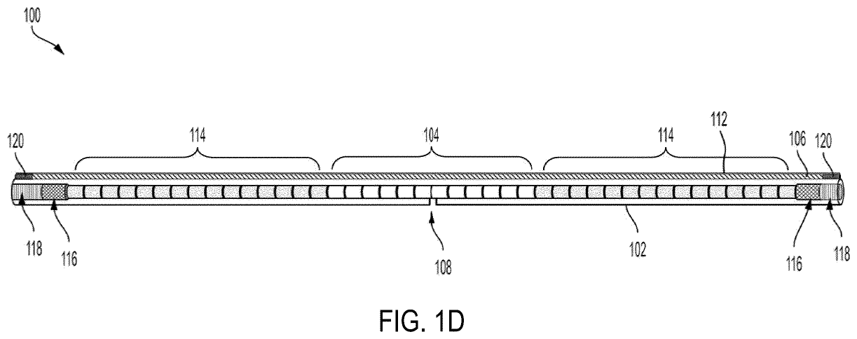

As described herein, administering gemcitabine can include the use of an intravesical system that comprises the gemcitabine and is insertable into the bladder for extended or sustained release of the gemcitabine. The terms “intravesical system”, “intravesical releasing system”, and “intravesical delivery system” may be used interchangeably. An exemplary intravesical system 100 is shown in and described below with respect to FIGS. 1A-1D and may otherwise be referred to herein as “TAR-200”, “intravesical gemcitabine releasing system”, “gemcitabine intravesical system”, or “INLEXZO™”. When inserted into the bladder, the intravesical system is submerged and freely mobile in the urine during the indwelling period. Said differently, the intravesical system does not float in urine, but rather has neutral buoyancy in urine so that it freely moves around throughout the urine in the bladder lumen.

INLEXZO™ is a nucleoside metabolic inhibitor-containing intravesical system, indicated for the treatment of adult patients with Bacillus Calmette Guérin (BCG)-unresponsive, non-muscle invasive bladder cancer (NMIBC) with carcinoma in situ (CIS) with or without papillary tumors.

In some embodiments, the intravesical system is a sterile, non-resorbable, flexible, intravesical system containing 256 mg gemcitabine HCL (equivalent to 225 mg gemcitabine free base). Once administered, the intravesical system retains its bi-oval shape and is free-moving within the bladder.

Gemcitabine HCL (a nucleoside metabolic inhibitor) is also called 2′-deoxy-2′,2′-difluorocytidine monohydrochloride (B-isomer). The molecular formula for gemcitabine HCL is C₉H₁₁F₂N₃O₄·HCl, the molecular weight is 299.66, and the structural formula is:

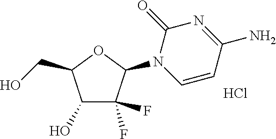

225 mg of gemcitabine is equal to about 256 mg of Gemcitabine HCL as shown below

$$225 mg gem ( \frac{1 g gem}{1000 mg gem} ) ( \frac{1 mol gem}{263.2 g gem} ) ( \frac{1 mol gem HCl}{1 mol gem} ) ( \frac{299.66 g gem FBE}{1 mol gem FBE} ) ( \frac{1000 mg gem FBE}{1 g gem FBE} ) = 256.01 mg gem HCl$$

In some embodiments, the intravesical drug delivery system comprises an almost white to light pink-brown colored gemcitabine component at the center surrounded on each side by off white to light blue-colored osmotic components. In some embodiments, the intravesical system comprises a drug constituent including a drug core comprising gemcitabine minitablets (which may appear as solid material) and osmotic minitablets (for osmotic performance). The gemcitabine minitablets and osmotic minitablets are contained within the bi-oval shaped device constituent. The color of the minitablets may vary but that has no effect on the potency of the intravesical system. In some embodiments, the gemcitabine component is an almost white to light pink-brown color. In some embodiments, the osmotic components are an off white to light blue-color. The minitablets (which may appear fused) contain the following ingredients:

Gemcitabine component (minitablets): containing a total dose of about 225 mg of the active ingredient gemcitabine (about 256 mg gemcitabine HCL), and the following inactive ingredients: polyethylene glycol 8000 (about 5 mg to about 12 mg), povidone K30 (about 10 mg to about 20 mg), and urea (about 35 mg to about 50 mg).

In some embodiments, the total of all gemcitabine minitablets in the intravesical system contain about 5 mg to about 12 mg polyethylene glycol 8000. In some embodiments, the gemcitabine minitablets contain about 6 mg to about 11 mg polyethylene glycol 8000. In some embodiments, the gemcitabine minitablets contain about 6.5 mg to about 10 mg polyethylene glycol 8000. In some embodiments, the gemcitabine minitablets contain about 7 mg to about 9 mg polyethylene glycol 8000. In some embodiments, the gemcitabine minitablets contain about 8 mg polyethylene glycol 8000.

In some embodiments, the total of all gemcitabine minitablets in the intravesical system contain about 10 mg to about 20 mg povidone K30. In some embodiments, the gemcitabine minitablets contain about 10.5 mg to about 17 mg povidone K30. In some embodiments, the gemcitabine minitablets contain about 11 mg to about 15 mg povidone K30. In some embodiments, the gemcitabine minitablets contain about 12.5 mg to about 14 mg povidone K30. In some embodiments, the gemcitabine minitablets contain about 13.4 mg povidone K30.

In some embodiments, the total of all gemcitabine minitablets in the intravesical system contain about 35 mg to about 50 mg urea. In some embodiments, the gemcitabine minitablets contain about 39 mg to about 47 mg urea. In some embodiments, the gemcitabine minitablets contain about 40 mg to about 45 mg urea. In some embodiments, the gemcitabine minitablets contain about 41.5 mg to about 43.5 mg urea. In some embodiments, the gemcitabine minitablets contain about 42.6 mg urea.

In some embodiments, the total of all gemcitabine minitablets in the intravesical system contain a total dose of about 225 mg of the active ingredient gemcitabine (about 256 mg gemcitabine HCl), and the following inactive ingredients: polyethylene glycol 8000 (about 8 mg), povidone K30 (about 13.4 mg), and urea (about 42.6 mg).

In some embodiments, each gemcitabine minitablet comprises about 19 mg to about 24 mg gemcitabine HCL, about 0.5 mg to about 1.5 mg Povidone 30, about 0.4 mg to about 1 mg polyethylene glycol (PEG) 8000, and about 2 mg to about 5 mg urea. Such values can also be expressed as percent weight, such that each gemcitabine minitablet comprises: about 72% to about 89% w/w gemcitabine HCL; about 1.6% to about 6.6% w/w Povidone 30; about 1.3% to about 4.4% w/w polyethylene glycol (PEG) 8000; and about 7% to about 20% w/w urea.

In some embodiments, each gemcitabine minitablet comprises about 21.36 mg gemcitabine HCL, about 1.12 mg Povidone 30, about 0.67 mg polyethylene glycol (PEG) 8000, and about 3.55 mg urea. Such values can also be expressed as percent weight, such that each gemcitabine minitablet comprises: about 80% w/w gemcitabine HCL; about 4.2% w/w Povidone 30; about 2.5% w/w polyethylene glycol (PEG) 8000; and about 13.3% w/w urea.

In some embodiments, the intravesical device further comprises osmotic components (urea minitablets) containing no active ingredient, and the following inactive ingredients: urea (about 630 mg to about 670 mg), polyethylene oxide 600,000 (about 50 mg to about 90 mg), and colorant, such as FD&C Blue No. 1 (about 0.0020 mg to about 0.0060 mg). In some embodiments, each osmotic minitablet comprises about 15 mg to about 25 urea, about 1 mg to about 4 mg polyethylene oxide (PEO), and about 0.0005 mg to about 0.0020 mg colorant, such as FD&C Blue No. 1. Such values can also be expressed as percent weight, such that each osmotic minitablet comprises about 79% to about 96% w/w urea; about 4% to about 20% w/w polyethylene oxide (PEO); and about 0.0001% to about 0.0010% w/w colorant, such as FD&C Blue No. 1.

In some embodiments, the osmotic (urea) minitablets comprise urea (about 648 mg), polyethylene oxide 600,000 (about 72 mg), and colorant (e.g., FD&C Blue No. 1) (about 0.0042 mg). In some embodiments, each osmotic minitablet comprises about 21.6 mg urea, about 2.40 mg polyethylene oxide (PEO), and about 0.0014 mg colorant, such as FD&C Blue No. 1. Such values can also be expressed as percent weight, such that each osmotic minitablet comprises about 90% w/w urea; about 10% w/w polyethylene oxide (PEO); and about 0.0006% w/w colorant, such as FD&C Blue No. 1.

In some embodiments, the intravesical device comprises between about 30-50, e.g., about 35-45, about 38-42, or about 42 total minitablets. In some embodiments, the intravesical device comprises about 25-35, e.g., about 28-32 or about 30 total osmotic minitablets. In some embodiments, the intravesical device comprises about 8-15, e.g., about 10-14 or about 12 gemcitabine minitablets.

In some embodiments, the gemcitabine minitablets have a diameter of about 2.4 mm to about 2.8 mm. In some embodiments, the gemcitabine minitablets have a diameter of about 2.63 mm. In some embodiments, the average thickness (i.e., length) of the gemcitabine minitablets is less than or equal to about 3.4 mm, including about 3.4 mm.

In some embodiments, the osmotic (urea) minitablets have a diameter of about 2.4 mm to about 2.8 mm. In some embodiments, the osmotic (urea) minitablets have a diameter of about 2.63 mm. In some embodiments, the average thickness (i.e., length) of the osmotic (urea) minitablets is less than or equal to about 3.5 mm, including about 3.5 mm.

TAR-200 includes a device constituent. The device constituent can include a bi-oval shaped tube. In some embodiments, the bi-oval shaped tube consists of a dual lumen silicone part (a silicone tube containing two lumens), a superelastic nitinol wire in a predefined shape (wireform), silicone spacers, and silicone adhesive. The minitablets are surrounded by a semipermeable membrane that contains a single delivery orifice.

Referring to FIGS. 1A-D, the intravesical system 100, shown as TAR-200, can include a bi-oval shaped tube or housing 102 that contains the gemcitabine 104. The housing 102 can be flexible for insertion and removal of the intravesical system 100 into and from the bladder. For example, the housing 102 may be composed of a biocompatible elastomeric polymer (e.g., silicone) that has the desired mechanical properties for manipulating the intravesical system 100. The housing 102 may be elastically deformable between a retention shape (e.g., a coiled or bi-oval shape, shown in FIG. 1A) for retaining the intravesical system in the individual's bladder and a deployment shape (e.g., an uncoiled shape, shown in FIG. 1D) for passing the intravesical system through the individual's urethra (e.g., during insertion and removal of the intravesical system 100). For example, the intravesical system 100 can include a retention frame 106 disposed within the housing 102 that has a predefined shape to retain the system in its bi-oval shape once inserted into the bladder. In some examples, the retention frame 106 is a super-elastic metal alloy wire, such as a nitinol wire. Once inserted into the bladder, the intravesical system 100 may be freely mobile within the bladder (e.g., within the urine in the bladder).

As illustrated in FIG. 1C, the housing 102 can include a dual lumen composed of a drug reservoir lumen 110 (e.g., a large lumen) that contains the gemcitabine 104 and a retention frame lumen 112 (e.g., a smaller lumen) that contains the retention frame 106. The drug reservoir lumen 110 may have an inner diameter between about 2 mm and 3 mm. The retention frame lumen 112 may have an inner diameter between about 0.4 mm and 1 mm. Although illustrated as two separate but attached (e.g., monolithic) circular portions in FIG. 1C, it is to be understood that the housing 102 may embody any reasonable shape that includes a dual lumen to separately contain the gemcitabine 104 and the retention frame 106. For example, the outer-most walls of the housing 102 may have a substantially circular or ovular shape which includes the dual lumen.

The intravesical system 100 may comprise about 225 mg of gemcitabine. In some examples, the intravesical system can comprise about 256 mg of gemcitabine hydrochloride (HCl) (equivalent to about 225 mg gemcitabine free base). The gemcitabine 104 contained within the intravesical system 100 may be in a non-liquid form. For example, the gemcitabine may be in the form of minitablets. As shown in FIGS. 1A and 1D, the gemcitabine minitablets 104 can be serially arranged within the housing 102.

Referring to FIGS. 1C and 1D, the intravesical system 100 can include a single delivery orifice 108 that controllably releases the gemcitabine 104 from the drug reservoir lumen 110 through the delivery orifice 108. The delivery orifice 108 may be a laser-drilled delivery orifice. In some examples, the diameter of the delivery orifice 108 can be between about 25-300 μm, such as between about 50-250 μm, or about 100-200 μm (e.g., about 150 μm). In FIG. 1A, the delivery orifice 108 is positioned centrally between the two ovals making up the bi-oval shape of the system for maximum, controlled release of the gemcitabine 104 from the delivery orifice 108, as described in greater detail below.

The intravesical system 100 may be configured to release the gemcitabine 104 into the urine in the bladder by osmotic pressure generated by an osmotic agent 114 (e.g., urea) contained within the housing 102 of the intravesical system 100. For example, the drug reservoir lumen 110 may contain active minitablets 104 that contain the gemcitabine HCL and osmotic minitablets 114 that contain the osmotic agent. As shown in FIG. 1A, the housing 102 may contain a plurality of the active minitablets 104 and a plurality of the osmotic minitablets 114. For example, the housing 102 may contain between about 30-50, e.g., about 35-45, about 38-42, or about 42 total minitablets. In some examples, the housing 102 may contain about 25-35, e.g., about 28-32 or about 30 total osmotic minitablets. In some examples, the housing may contain about 8-15, e.g., about 10-14 or about 12 active minitablets. The minitablets (including the active minitablets and the osmotic minitablets) may be arranged serially within the housing 102 to form a drug core, as shown at least in FIG. 1D. In the arrangement illustrated in FIG. 1D, the plurality of active minitablets 104 are arranged with their longitudinal midpoint located approximately over the release orifice 108. The osmotic minitablets 114 are arranged in two equal groups flanked on either side of the active minitablets 104. As a result, the plurality of active minitablets 104 are centered about the release orifice 108 with an equal number of the osmotic minitablets 114 located on either side of the plurality of active minitablets 104. This arrangement of active minitablets 104 and osmotic minitablets 114 may be beneficial to controlling and/or maximizing osmotic release of the gemcitabine from the delivery orifice 108.

As shown in FIGS. 1A-1B, the end portions 150 of the intravesical system 100 can be sealed. For example, as shown, each end portion 150 can include a spacer 116, such as a silicone spacer or other material that is impermeable to gemcitabine, that closes or plugs each end 150 of the drug reservoir lumen 110. In some embodiments, the opposed ends 150 of the retention frame 106 can overlay the respective spacers 116. In some examples, adhesive 118, such as silicone adhesive or other material impermeable to gemcitabine, can be injected into the ends 150 of the drug reservoir lumen 110 to secure the silicone spacers 116 within the ends 150 of the drug reservoir lumen 110. Separately, the retention frame lumen 112 can be sealed using an adhesive, such as silicone adhesive 120. Once the adhesives 118, 120 are injected into the ends 150 of the housing 102, any extra housing 102 and/or adhesive 118, 120 at the ends can be trimmed.

The drug reservoir lumen 110, as well as the spacers 116 and adhesive 118 located at the opposed ends 150 of the drug reservoir lumen 110, can form a semi-permeable membrane that is permeable to water/urine and impermeable to the gemcitabine and osmotic agent 114 contained therein. In this manner, substantially no amount of the solubilized gemcitabine or the osmotic agent can diffuse through the housing 102 over the dosing cycle in which the intravesical system 100 is indwelling in the bladder. Accordingly, when indwelling the bladder, the intravesical system 100 can operate as an osmotic pump. Water or urine in the individual's bladder can diffuse through the housing 102 into the drug reservoir lumen 110 to contact and solubilize the active minitablets 104 and osmotic minitablets 114, creating an osmotic pressure to drive the solubilized gemcitabine from the intravesical system (drug reservoir lumen 110) through delivery orifice 108. Additional details about the intravesical system can be found in U.S. Pat. No. 10,729,823, the entire contents of which is expressly incorporated herein by reference.

In some examples, the active minitablets 104 including the gemcitabine may further include an osmotic agent (i.e., in addition to the osmotic minitablets 114 including urea). The osmotic agent may include urea. In some examples, each active minitablet 104 may include at least 50 percent by weight gemcitabine HCL, at least 60 percent by weight gemcitabine HCL, at least 75 percent by weight gemcitabine HCL, from about 60 to about 99 percent by weight gemcitabine HCL, or from about 75 to about 95 percent by weight gemcitabine HCL.

In some examples, each osmotic minitablet 114 may include at least 80 percent by weight osmotic agent, e.g., urea, at least 85 percent by weight osmotic agent, at least 90 percent by weight osmotic agent, from about 80 to about 99 percent by weight osmotic agent, or from about 85 to about 95 percent by weight osmotic agent. The remainder of the active minitablets 104 and/or osmotic minitablets 114 may include excipients such as pharmaceutical lubricants, stabilizing agents, or binding agents, for example oil-based lubricants, polyethylene glycol (PEG), or polyvinylpyrrolidone (PVP). The excipients may also include a viscosity enhancing (e.g., release delay) agent, for example polyethylene oxide (PEO). For example, a viscosity enhancing agent could be provided in a portion of the active minitablets 104, a portion of the osmotic minitablets 114, or both the active minitablets 104 and osmotic minitablets 114 to further control release of the gemcitabine. In some embodiments, the active minitablets 104 comprise about 72% to about 89% w/w gemcitabine HCL; about 1.6% to about 6.6% w/w Povidone 30; about 1.3% to about 4.4% w/w polyethylene glycol (PEG) 8000; and about 7% to about 20% w/w urea. In some embodiments, the active minitablets 104 comprise 80% by weight gemcitabine HCL, 13.3% by weight urea, 2.5% by weight polyethylene glycol (PEG), and 4.2% by weight polyvinylpyrrolidone. In some embodiments, the PEG comprises PEG 8000.

As noted above, the intravesical system 100 may be flexible between a bi-oval, or coiled, shape (retention shape), and an uncoiled shape (insertion shape). As shown in FIG. 1A, in the coiled shape, the intravesical system 100 may have a maximum width “X” that extends the width of both ovals and a maximum height “Y” that extends the height of an oval and is perpendicular to the maximum width X. In some embodiments, the intravesical system 100 has a maximum width of about 4-7 cm, about 4.5-6.5 cm, or about 5-6 cm (e.g., 5.5 cm or 6 cm). In some embodiments, the intravesical system 100 has a maximum height of about 3-6 cm, about 3.5-5.5 cm, or about 4-5 cm (e.g., about 4.5 cm or 5 cm). In some embodiments, when in the retention shape, the intravesical system defines a bi-oval coiled shape having a maximum width and a maximum height perpendicular to the maximum width, wherein the maximum width is about 4-7 cm and the maximum height is about 3-6 cm. In some embodiments, the maximum width is about 5.5 cm and the maximum height is about 4.5 cm. As shown in FIG. 1D, in the uncoiled shape, the intravesical system 100 may have a length between about 15-20 cm, such as about 16-19 cm or about 17-18 cm (e.g., 17 cm).

The intravesical system 100 can be deployed into the bladder through the working channel of a catheter, cystoscope, or other deployment instrument positioned in the urethra. Alternatively, the intravesical system 100 can be inserted with a urinary placement catheter that facilitates deployment of the intravesical system 100 into the bladder of an individual.

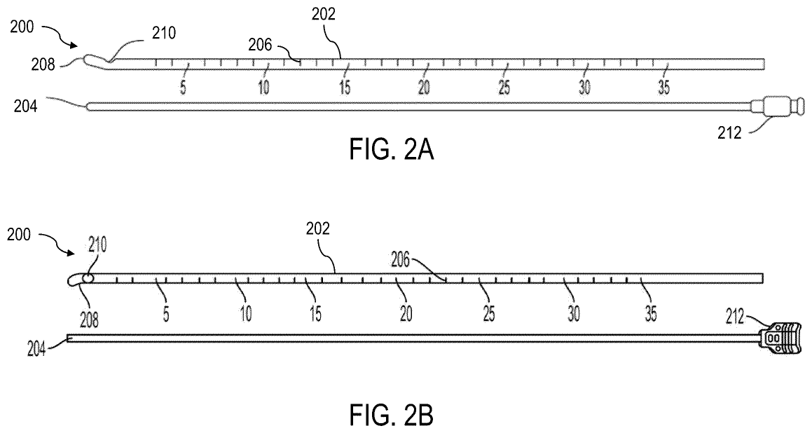

FIGS. 2A-2B show two different versions of an exemplary inserter 200 for the transurethral placement of the intravesical system, TAR-200, into the bladder (not shown). The inserter 200, or “Urinary Placement Catheter,” or “UPC,” can comprise a catheter shaft (also referred to as the urinary catheter) 202 and stylet 204 that fits within the inner channel of the catheter shaft 202. According to some embodiments, the catheter shaft 202 can be clear and the stylet 204 can be colored (e.g., a green stylet). In some embodiments, the catheter shaft 202 can comprises depth markings 206 that can be used to measure the extent to which the catheter shaft 202 is inserted into the urethra. The depth marking 206 can also be aligned with the upward direction of the distal coude tip 208 to aid the health care provider in orienting the distal coudé tip 208 within the bladder. The catheter shaft 202 can include an exit port 210. The healthcare provider can push the TAR-200 through the catheter shaft 202 with the stylet 204 until the TAR-200 exits the catheter shaft 202 via the exit port 210 to deploy TAR-200 into the bladder. The stylet 204 can further comprise a stylet hub 212 that serves as a gripping surface for manipulation of the stylet 204 by the user. In some embodiments, the stylet hub 212 is translucent. In other embodiments, the stylet hub 212 is colored (e.g., green). Additional embodiments and/or details of the inserter 200, as well as additional description of its usage, are described in U.S. Pat. No. 10,064,980, which is incorporated herein by reference in its entirety.

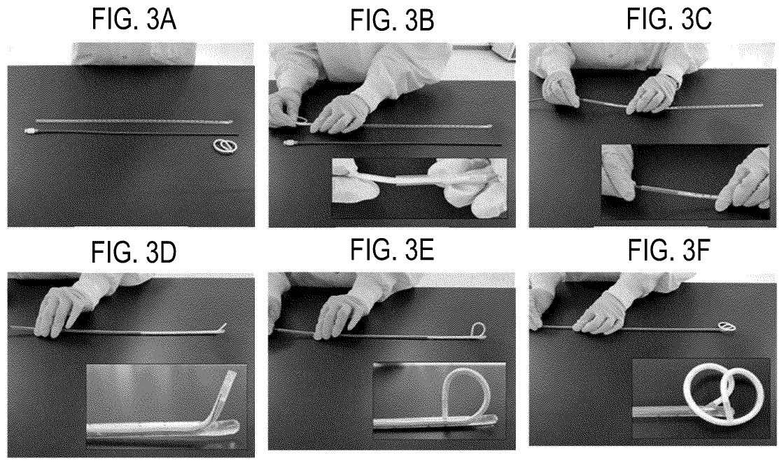

In FIG. 3A, TAR-200 is depicted next to an exemplary urinary placement catheter (UPC) and in the retention shape, suited to retain the system within the bladder. The urinary placement catheter can be inserted into the urethra by a healthcare provider until the distal tip of the catheter shaft enters into the bladder (not shown). FIG. 3B shows a healthcare provider manually uncoiling TAR-200 from its native bi-oval shape and loading TAR-200 into the proximal end of the catheter shaft of the urinary placement catheter. FIG. 3C shows the healthcare provider inserting the stylet into the catheter shaft to advance TAR-200 through the catheter shaft. FIG. 3D shows the stylet manually advancing TAR-200 through the catheter shaft and TAR-200 beginning to emerge from the exit port of the catheter shaft. FIG. 3E shows TAR-200 as it emerges from the exit port of the catheter shaft as it is deployed into the bladder lumen (not depicted). FIG. 3F shows TAR-200 just prior to complete deployment from the catheter shaft into the bladder (not depicted), where TAR-200 returns to its original bi-oval form. The intravesical system 100 can be removed from the bladder transurethrally via cystoscopy and endoscopic graspers.

Also provided herein are kits comprising an intravesical system comprising about 256 mg gemcitabine HCL. A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert.

In one embodiment, a label is used to indicate that the contents are to be used for a specific therapeutic application. The label also indicates directions for use of the contents, such as in the methods described herein.

In certain embodiments, the pharmaceutical compositions are presented in a pack or carton which contains one or more intravesical systems containing one or more compounds provided herein, e.g., TAR-200. In one embodiment, the pack or carton is accompanied by instructions for administration. In one embodiment, the pack or carton is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the product insert. In one embodiment, intravesical systems containing one or more compounds provided herein, e.g., TAR-200 are prepared, placed in an appropriate container, and labeled for treatment of an indicated condition.

In some embodiments, the kit further comprises the inserter (e.g., UPC). In some embodiments, the intravesical system and the inserter are packaged in separate pouches (e.g., Tyvek pouches). In some embodiments, the intravesical system and the inserter are packaged together in the same pouch (e.g., a Tyvek pouch).

In some embodiments, the package insert provides instructions for insertion and/or removal of the intravesical system. In some embodiments, the package insert provides instructions to insert the intravesical system into the bladder via transurethral catheterization using the co-packaged UPC. In some embodiments, the kit further comprises instructions for inserting the intravesical system into the bladder of an individual, and/or instructions for removing the intravesical drug delivery system. In some embodiments, the instructions require use of water-based lubricant, syringes, non-cutting, grasping forceps, and/or a flexible or rigid cystoscope. In some embodiments, the water-based lubricant, syringes, non-cutting, grasping forceps, and/or a flexible or rigid cystoscope are not included in the kit (i.e., are provided separately).

### EMBODIMENTS

The following embodiments are exemplary and are not intended to limit the scope of any invention described herein.

1. A method of treating bladder cancer in an individual comprising inserting an intravesical system comprising about 256 mg of gemcitabine HCL into the bladder of the individual once about every three weeks for at least 24 weeks, wherein each intravesical system is removed from the bladder about three weeks after insertion into the bladder and wherein each intravesical system is removed prior to insertion of another intravesical system, wherein each intravesical system provides sustained release of gemcitabine into bladder urine, wherein about 65% to about 85% of the gemcitabine is released over about the first 7 days after insertion into the bladder and about 85% to about 100% of the gemcitabine is released over about 21 days after insertion into the bladder, and wherein the intravesical system comprises gemcitabine minitablets comprising a total of: i) about 256 mg gemcitabine HCL; ii) about 10 mg to about 20 mg Povidone 30; iii) about 5 mg to about 12 mg polyethylene glycol (PEG) 8000; and iv) about 35 mg to about 50 mg urea.

2. The method of embodiment 1, wherein about 70% of the gemcitabine is released over about the first 7 days after insertion into the bladder and about 90% of the gemcitabine is released over about 21 days after insertion into the bladder, and wherein the percent of gemcitabine released is estimated using an in vitro release model.

3. The method of embodiment 1, wherein about 76.9% of the gemcitabine is released over about the first 7 days after insertion into the bladder and about 99% of the gemcitabine is released over about 21 days after insertion into the bladder, and wherein the percent of gemcitabine released is estimated using in vivo data.

4. The method of embodiment 3, wherein the in vivo data comprise urine gemcitabine related component (GRC) amount-time profiles, wherein the percent of gemcitabine released is estimated using nonlinear mixed effect modeling.

5. A method of delivering gemcitabine to an individual having bladder cancer, the method comprising inserting an intravesical system comprising about 256 mg gemcitabine HCL into the bladder of the individual to target a pharmacokinetic (PK) profile for gemcitabine, wherein the PK profile has: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose; and wherein the intravesical system comprises gemcitabine minitablets comprising a total of: i) about 256 mg gemcitabine HCL; ii) about 10 mg to about 20 mg Povidone 30; iii) about 5 mg to about 12 mg polyethylene glycol (PEG) 8000; and iv) about 35 mg to about 50 mg urea.

6. The method of any one of embodiments 1-5, wherein the individual has non-muscle invasive bladder cancer (NMIBC).

7. The method of embodiment 6, wherein the individual has Bacillus Calmette Guerin (BCG)-unresponsive, high-risk, non-muscle invasive bladder cancer (HR-NMIBC) with carcinoma in situ (CIS) with or without papillary tumors.

8. The method of any one of embodiments 1-7, wherein each intravesical system produces a systemic exposure of gemcitabine in the individual of less than about 0.1 μg/mL.

9. The method of any one of embodiments 1-8, wherein each intravesical system produces a maximum mean amount of about 31.5 mg gemcitabine related components (GRC) in urine on Day 3.

10. The method of any one of embodiments 1-9, wherein each intravesical system releases about 2% to about 20% of the gemcitabine on each of days 1-7 after the intravesical system is inserted into the bladder of the individual.

11. The method of any one of embodiments 1-10, wherein each intravesical system produces a PK profile having a mean excreted GRC of about 2 mg to about 21 mg on day 1 after insertion in the bladder, about 17 mg to about 37 mg on day 2 after insertion in the bladder, about 20 mg to about 40 mg on day 3 after insertion into the bladder, about 12 mg to about 38 mg on day 4 after insertion into the bladder, about 5 mg to about 35 mg on day 5 after insertion into the bladder, about 2 mg to about 27 mg on day 6 after insertion into the bladder, and/or about 1 mg to about 20 mg on day 7 after insertion into the bladder.

12. The method of any one of embodiments 1-11, wherein each intravesical system produces an average urine concentration of about 2 μg/mL to about 15 g/mL on about days 1-7 after insertion into the bladder of the individual.

13. The method of any one of embodiments 1-12, wherein each gemcitabine minitablet comprises: i) about 19 mg to about 24 mg gemcitabine HCL; ii) about 0.5 mg to about 1.5 mg Povidone 30; iii) about 0.4 mg to about 1 mg polyethylene glycol (PEG) 8000; and iv) about 2 mg to about 5 mg urea.

14. The method of embodiment 13, wherein each gemcitabine minitablet comprises: i) about 21.36 mg gemcitabine HCL; ii) about 1.12 mg Povidone 30; iii) about 0.67 mg polyethylene glycol (PEG) 8000; and iv) about 3.55 mg urea.

15. The method of any one of embodiments 1-14, wherein each gemcitabine minitablet comprises: i) about 72% to about 89% w/w gemcitabine HCL; ii) about 1.6% to about 6.6% w/w Povidone 30; iii) about 1.3% to about 4.4% w/w polyethylene glycol (PEG) 8000; and iv) about 7% to about 20% w/w urea.

16. The method of embodiment 15, wherein each gemcitabine minitablet comprises: i) about 80% w/w gemcitabine HCL; ii) about 4.2% w/w Povidone 30; iii) about 2.5% w/w polyethylene glycol (PEG) 8000; and iv) about 13.3% w/w urea.

17. The method of any one of embodiments 1-16, wherein the gemcitabine minitablets of each intravesical system comprise a total of: i) about 256.3 mg gemcitabine HCL; ii) about 13.4 mg Povidone 30; iii) about 8 mg polyethylene glycol (PEG) 8000; and iv) about 42.6 mg urea.

18. The method of any one of embodiments 1-17, wherein each intravesical system comprises osmotic minitablets comprising: i) about 15 mg to about 25 mg urea; and ii) about 1 mg to about 4 mg polyethylene oxide (PEO).

19. The method of embodiment 18, wherein each intravesical system comprises osmotic minitablets comprising: i) about 21.6 mg urea; and ii) about 2.4 mg polyethylene oxide (PEO).

20. The method of any one of embodiments 1-17, wherein each intravesical system comprises osmotic minitablets comprising: i) about 79% to about 96% w/w urea; and ii) about 4% to about 20% w/w polyethylene oxide (PEO).

21. The method of embodiment 20, wherein each intravesical system comprises osmotic minitablets comprising: i) about 90% w/w urea; and ii) about 10% w/w polyethylene oxide (PEO).

22. The method of any one of embodiments 18-21, wherein the osmotic minitablets of each intravesical system comprise a total of: i) about 630 mg to about 670 mg urea; and ii) about 50 mg to about 90 mg polyethylene oxide (PEO).

23. The method of embodiment 22, wherein the osmotic minitablets of each intravesical system comprise a total of: i) about 648 mg urea; and ii) about 72 mg polyethylene oxide (PEO).

24. The method of any one of embodiments 18-23, wherein the osmotic minitablets further comprise colorant.

25. The method of embodiment 24, wherein the colorant is FD&C Blue No. 1 colorant.

26. The method of any one of embodiments 1-25, wherein each intravesical system comprises a flexible bi-oval shaped tube.

27. The method of any one of embodiments 1-26, wherein when in the retention shape, the intravesical system defines a bi-oval coiled shape having a maximum width and a maximum height perpendicular to the maximum width, wherein the maximum width is about 4-7 cm and the maximum height is about 3-6 cm.

28. The method of embodiment 27, wherein the maximum width is about 5.5 cm and the maximum height is about 4.5 cm.

29. The method of any one of embodiments 1-28, wherein each intravesical system comprises 12 gemcitabine minitablets and 30 osmotic agent minitablets.

30. The method of any one of embodiments 1-29, wherein each intravesical system comprises: a) 12 gemcitabine minitablets comprising a total of: i) about 256.3 mg gemcitabine HCL; ii) about 13.4 mg Povidone 30; iii) about 8 mg polyethylene glycol (PEG) 8000; and iv) about 42.6 mg urea; and b) 30 osmotic agent minitablets comprising a total of: i) about 648 mg urea; and ii) about 72 mg polyethylene oxide (PEO).

31. The method of any one of embodiments 1-30, wherein each intravesical system comprises an orifice with an about 25 micron to about 300 micron diameter.

32. The method of embodiment 31, wherein each intravesical system comprises an orifice with an about 150 micron diameter.

33. The method of any one of embodiments 1-32, wherein each intravesical system is configured to be freely mobile in the bladder of the individual.

34. The method of any one of embodiments 1-33, wherein the individual is ineligible for or has elected not to undergo radical cystectomy.

35. A method of treating Bacillus Calmette Guerin (BCG)-unresponsive, high-risk, non-muscle invasive bladder cancer (HR-NMIBC) with carcinoma in situ (CIS) with or without papillary tumors in an individual, the method comprising inserting an intravesical system comprising about 256 mg of gemcitabine hydrochloride into the bladder once every 3 weeks up to 6 months (8 doses), followed by once every 12 weeks for up to 18 months, or until persistent or recurrent HR-NMIBC, disease progression, or unacceptable toxicity.

36. The method of embodiment 35, comprising a total of 14 doses over 2 years of therapy.

37. An intravesical system comprising: a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 256 mg gemcitabine HCl; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

38. An intravesical system comprising gemcitabine minitablets comprising a total of: i) about 256 mg gemcitabine HCL; ii) about 10 mg to about 20 mg Povidone 30; iii) about 5 mg to about 12 mg polyethylene glycol (PEG) 8000; and iv) about 35 mg to about 50 mg urea, wherein, when tested using an in vitro release model, the intravesical system releases about 70% of the gemcitabine over about 7 days and about 90% of the gemcitabine about 21 days.

39. The intravesical system of embodiment 37-38, wherein, when the intravesical system is indwelling in the bladder of an individual, the intravesical system generates a pharmacokinetic (PK) profile having: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose.

40. The intravesical system of any one of embodiments 37-39, further comprising spacers at each end of the housing.

41. The intravesical system of embodiment 40, wherein an adhesive secures the spacers within the ends of the drug reservoir lumen.

42. The intravesical system of any one of embodiments 37-41, wherein the gemcitabine minitablets comprise a total of: i) about 256.3 mg gemcitabine HCL; ii) about 10 mg to about 20 mg Povidone 30; iii) about 5 mg to about 12 mg polyethylene glycol (PEG) 8000; and iv) about 35 mg to about 50 mg urea.

43. The intravesical system of embodiment 42, wherein the gemcitabine minitablets comprise a total of: i) about 256.3 mg gemcitabine HCl; ii) about 13.4 mg Povidone 30; iii) about 8 mg polyethylene glycol (PEG) 8000; and iv) about 42.6 mg urea.

44. The intravesical system of any one of embodiments 37-43, wherein the osmotic minitablets comprise a total of: i) about 630 mg to about 670 mg urea; and ii) about 50 mg to about 90 polyethylene oxide (PEO).

45. The intravesical system of embodiment 44, wherein the osmotic minitablets comprise a total of: i) about 648 mg urea; and ii) about 72 mg polyethylene oxide (PEO).

46. The intravesical system of embodiment 44 or 45, wherein the osmotic minitablets further comprise FD&C Blue No. 1 colorant.

47. The intravesical system of any one of embodiments 37-46, wherein the intravesical system is configured as a flexible bi-oval shaped tube.

48. The intravesical system of any one of embodiments 37-47, wherein when in the retention shape, the intravesical system defines a bi-oval coiled shape having a maximum width and a maximum height perpendicular to the maximum width, wherein the maximum width is about 4-7 cm and the maximum height is about 3-6 cm.

49. The intravesical system of embodiment 48, wherein the maximum width is about 5.5 cm and the maximum height is about 4.5 cm.

50. The intravesical system of any one of embodiments 37-49, wherein the intravesical system is sterile.

51. The intravesical system of any one of embodiments 37-50, wherein the intravesical system is configured to freely mobile in a bladder of an individual.

52. The method of any one of embodiments 1-36, wherein such treatment results in a) a complete response (CR) rate of about 74% to about 91% in a population of patients who have received such treatment; b) a median duration of response (DoR) of about 9 months to about 30 months in a population of patients who have received such treatment; c) a 12-month complete response (CR) rate of about 40% to about 50% in a population of patients who have received such treatment; and/or d) a 12-month duration of response (DoR) rate of about 43% to about 67% in a population of patients who have received such treatment.

53. The method of embodiment 52, wherein such treatment results in a) a complete response (CR) rate of about 84% in a population of patients who have received such treatment; b) a median duration of response (DoR) of about 25.8 months in a population of patients who have received such treatment; c) a 12-month complete response (CR) rate of about 45.9% in a population of patients who have received such treatment; d) a 12-month duration of response (DoR) rate of about 55.7% in a population of patients who have received such treatment; and/or e) a 12-month duration of response (DoR) rate of about 52.9% in a population of patients who have received such treatment.

54. A method of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a median duration of response (DoR) of about 9 months to about 30 months in a population of patients who have received such treatment.

55. The method of embodiment 54, wherein the median DoR is about 25.8 months.

56. A method of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a 12-month complete response (CR) rate of about 40% to about 50% in a population of patients who have received such treatment.

57. The method of embodiment 56, wherein the 12-month CR rate is about 45.9%.

58. A method of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a 12-month duration of response (DoR) rate of about 43% to about 67% in a population of patients who have received such treatment.

59. The method of embodiment 58, wherein the 12-month DoR rate is about 55.7%.

60. The method of embodiment 58, wherein the 12-month DoR rate is about 52.9%.

61. A method of treating Bacillus Calmette Guerin (BCG)-unresponsive, high-risk, non-muscle invasive bladder cancer (HR-NMIBC) with carcinoma in situ (CIS) with or without papillary tumors in an individual comprising inserting an intravesical system comprising about 256 mg of gemcitabine HCL into the bladder of the individual once every three weeks for at least 24 weeks, wherein such treatment results in a median time to onset of response of about 2 months to about 9 months in a population of patients who have received such treatment.

62. The method of embodiment 61, wherein the median time to onset of response is about 2.8 months.

63. The method of any one of embodiments 54-62, further comprising inserting an intravesical system comprising about 256 mg of gemcitabine HCL into the bladder of the individual once every twelve weeks for up to 18 months.

64. A kit comprising an intravesical system and instructions for use to treat Bacillus Calmette Guerin (BCG)-unresponsive, high-risk, non-muscle invasive bladder cancer (HR-NMIBC) with carcinoma in situ (CIS) with or without papillary tumors, the intravesical system comprising a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 256 mg gemcitabine HCL; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

65. The kit of embodiment 64, further comprising a urinary placement catheter adapted for insertion of the intravesical system into the bladder.

66. The kit of embodiment 64 or 65, wherein the intravesical system is sterile.

67. The method of any one of embodiments 1-36 or 52-63, wherein each intravesical system produces a mean urinary excretion on Days 2 through 5 of 21 mg to 32 mg per day as gemcitabine and dFdU.

68. The method of any one of embodiments 1-36 or 52-64, wherein each intravesical system comprises an almost white to light pink-brown colored gemcitabine component at the center surrounded on each side by off white to light blue-colored osmotic components.

69. The intravesical system of any one of embodiments 37-51, comprising an almost white to light pink-brown colored gemcitabine component at the center surrounded on each side by off white to light blue-colored osmotic components.

70. A method of treating bladder cancer in an individual comprising inserting an intravesical system comprising about 225 mg of gemcitabine into the bladder of the individual once about every three weeks for at least 24 weeks, wherein each intravesical system is removed from the bladder about three weeks after insertion into the bladder and wherein each intravesical system is removed prior to insertion of another intravesical system, wherein each intravesical system provides sustained release of gemcitabine into bladder urine, wherein about 65% to about 85% of the gemcitabine is released over about the first 7 days after insertion into the bladder and about 85% to about 100% of the gemcitabine is released over about 21 days after insertion into the bladder, and wherein the intravesical system comprises gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

71. The method of embodiment 70, wherein about 70% of the gemcitabine is released over about the first 7 days after insertion into the bladder and about 90% of the gemcitabine is released over about 21 days after insertion into the bladder, and wherein the percent of gemcitabine released is estimated using an in vitro release model.

72. The method of embodiment 70, wherein about 76.9% of the gemcitabine is released over about the first 7 days after insertion into the bladder and about 99% of the gemcitabine is released over about 21 days after insertion into the bladder, and wherein the percent of gemcitabine released is estimated using in vivo data.

73. The method of embodiment 72, wherein the in vivo data comprise urine gemcitabine related component (GRC) amount-time profiles, wherein the percent of gemcitabine released is estimated using nonlinear mixed effect modeling.

74. A method of delivering gemcitabine to an individual having bladder cancer, the method comprising inserting an intravesical system comprising about 225 mg gemcitabine into the bladder of the individual to target a pharmacokinetic (PK) profile for gemcitabine, wherein the PK profile has: a) a mean Cmax of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose; and wherein the intravesical system comprises gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

75. The method of any one of embodiments 70-74, wherein the individual has non-muscle invasive bladder cancer (NMIBC), optionally wherein the individual has Bacillus Calmette Guerin (BCG)-unresponsive NMIBC with carcinoma in situ (CIS) with or without papillary tumors.

76. The method of embodiment 75, wherein the individual has Bacillus Calmette Guerin (BCG)-unresponsive, high-risk, non-muscle invasive bladder cancer (HR-NMIBC) with carcinoma in situ (CIS) with or without papillary tumors.

77. The method of any one of embodiments 70-76, wherein each intravesical system produces a systemic exposure of gemcitabine in the individual of less than about 0.1 μg/mL.

78. The method of any one of embodiments 70-77, wherein each intravesical system produces a maximum mean amount of about 31.5 mg gemcitabine related components (GRC) in urine on Day 3.

79. The method of any one of embodiments 70-78, wherein each intravesical system releases about 2% to about 20% of the gemcitabine on each of days 1-7 after the intravesical system is inserted into the bladder of the individual.

80. The method of any one of embodiments 70-79, wherein each intravesical system produces a PK profile having a mean excreted GRC of about 2 mg to about 21 mg on day 1 after insertion in the bladder, about 17 mg to about 37 mg on day 2 after insertion in the bladder, about 20 mg to about 40 mg on day 3 after insertion into the bladder, about 12 mg to about 38 mg on day 4 after insertion into the bladder, about 5 mg to about 35 mg on day 5 after insertion into the bladder, about 2 mg to about 27 mg on day 6 after insertion into the bladder, and/or about 1 mg to about 20 mg on day 7 after insertion into the bladder.

81. The method of any one of embodiments 70-80, wherein each intravesical system produces an average urine concentration of about 2 μg/mL to about 15 μg/mL on about days 1-7 after insertion into the bladder of the individual.

82. The method of any one of embodiments 70-81, wherein each gemcitabine minitablet comprises: i) about 19 mg to about 24 mg gemcitabine HCL; ii) about 0.5 mg to about 1.5 mg Povidone 30; iii) about 0.4 mg to about 1 mg polyethylene glycol (PEG) 8000; and iv) about 2 mg to about 5 mg urea.

83. The method of embodiment 82, wherein each gemcitabine minitablet comprises: i) about 21.36 mg gemcitabine HCl; ii) about 1.12 mg Povidone 30; iii) about 0.67 mg polyethylene glycol (PEG) 8000; and iv) about 3.55 mg urea.

84. The method of any one of embodiments 70-83, wherein each gemcitabine minitablet comprises: i) about 72% to about 89% w/w gemcitabine HCL; ii) about 1.6% to about 6.6% w/w Povidone 30; iii) about 1.3% to about 4.4% w/w polyethylene glycol (PEG) 8000; and iv) about 7% to about 20% w/w urea.

85. The method of embodiment 84, wherein each gemcitabine minitablet comprises: i) about 80% w/w gemcitabine HCL; ii) about 4.2% w/w Povidone 30; iii) about 2.5% w/w polyethylene glycol (PEG) 8000; and iv) about 13.3% w/w urea.

86. The method of any one of embodiments 70-85, wherein the gemcitabine minitablets of each intravesical system comprise a total of: i) about 225 mg gemcitabine; ii) about 13.4 mg Povidone 30; iii) about 8 mg polyethylene glycol (PEG) 8000; and iv) about 42.6 mg urea.

87. The method of any one of embodiments 70-86, wherein each intravesical system comprises osmotic minitablets comprising: i) about 15 mg to about 25 mg urea; and ii) about 1 mg to about 4 mg polyethylene oxide (PEO).

88. The method of embodiment 87, wherein each intravesical system comprises osmotic minitablets comprising: i) about 21.6 mg urea; and ii) about 2.4 mg polyethylene oxide (PEO).

89. The method of any one of embodiments 70-86, wherein each intravesical system comprises osmotic minitablets comprising: i) about 79% to about 96% w/w urea; and ii) about 4% to about 20% w/w polyethylene oxide (PEO).

90. The method of embodiment 89, wherein each intravesical system comprises osmotic minitablets comprising: i) about 90% w/w urea; and ii) about 10% w/w polyethylene oxide (PEO).

91. The method of any one of embodiments 87-90, wherein the osmotic minitablets of each intravesical system comprise a total of: i) about 630 mg to about 670 mg urea; and ii) about 50 mg to about 90 mg polyethylene oxide (PEO).

92. The method of embodiment 91, wherein the osmotic minitablets of each intravesical system comprise a total of: i) about 648 mg urea; and ii) about 72 mg polyethylene oxide (PEO).

93. The method of any one of embodiments 87-92, wherein the osmotic minitablets further comprise colorant.

94. The method of embodiment 93, wherein the colorant is FD&C Blue No. 1 colorant.

95. The method of any one of embodiments 70-94, wherein each intravesical system comprises a flexible bi-oval shaped tube.

96. The method of any one of embodiments 70-95, wherein when in the retention shape, the intravesical system defines a bi-oval coiled shape having a maximum width and a maximum height perpendicular to the maximum width, wherein the maximum width is about 4-7 cm and the maximum height is about 3-6 cm.

97. The method of embodiment 96, wherein the maximum width is about 5.5 cm and the maximum height is about 4.5 cm.

98. The method of any one of embodiments 70-97, wherein each intravesical system comprises 12 gemcitabine minitablets and 30 osmotic agent minitablets.

99. The method of any one of embodiments 70-98, wherein each intravesical system comprises: a) 12 gemcitabine minitablets comprising a total of: i) about 225 mg gemcitabine; ii) about 13.4 mg Povidone 30; iii) about 8 mg polyethylene glycol (PEG) 8000; and iv) about 42.6 mg urea; and b) 30 osmotic agent minitablets comprising a total of: i) about 648 mg urea; and ii) about 72 mg polyethylene oxide (PEO).

100. The method of any one of embodiments 70-99, wherein each intravesical system comprises an orifice with an about 25 micron to about 300 micron diameter.

101. The method of embodiment 100, wherein each intravesical system comprises an orifice with an about 150 micron diameter.

102. The method of any one of embodiments 70-101, wherein each intravesical system is configured to be freely mobile in the bladder of the individual.

103. The method of any one of embodiments 70-102, wherein the individual is ineligible for or has elected not to undergo radical cystectomy.

104. A method of treating Bacillus Calmette Guerin (BCG)-unresponsive, non-muscle invasive bladder cancer (NMIBC) with carcinoma in situ (CIS) with or without papillary tumors in an individual, the method comprising inserting an intravesical system comprising about 225 mg of gemcitabine into the bladder once every 3 weeks up to 6 months (8 doses), followed by once every 12 weeks for up to 18 months, or until persistent or recurrent NMIBC, disease progression, or unacceptable toxicity.

105. The method of embodiment 104, comprising a total of 14 doses over 2 years of therapy.

106. An intravesical system comprising: a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 225 mg gemcitabine; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

107. An intravesical system comprising gemcitabine minitablets comprising a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea, wherein, when tested using an in vitro release model, the intravesical system releases about 70% of the gemcitabine over about 7 days and about 90% of the gemcitabine about 21 days.

108. The intravesical system of embodiment 106 or 107, wherein, when the intravesical system is indwelling in the bladder of an individual, the intravesical system generates a pharmacokinetic (PK) profile having: a) a mean Cₘₐₓ of gemcitabine of about 10 μg/mL to about 25 μg/mL; b) a Tmax of gemcitabine of about 2 days to about 4 days; and/or c) a mean cumulative excreted gemcitabine of about 30% to about 60%, wherein the mean cumulative excreted gemcitabine is the percentage of the administered dose.

109. The intravesical system of any one of embodiments 106-108, further comprising spacers at each end of the housing.

110. The intravesical system of embodiment 109, wherein an adhesive secures the spacers within the ends of the drug reservoir lumen.

111. The intravesical system of any one of embodiments 106-110, wherein the gemcitabine minitablets comprise a total of: i) about 225 mg gemcitabine; ii) about 10 mg to about 20 mg Povidone 30; iii) about 5 mg to about 12 mg polyethylene glycol (PEG) 8000; and iv) about 35 mg to about 50 mg urea.

112. The intravesical system of embodiment 111, wherein the gemcitabine minitablets comprise a total of: i) about 225 mg gemcitabine; ii) about 13.4 mg Povidone 30; iii) about 8 mg polyethylene glycol (PEG) 8000; and iv) about 42.6 mg urea.

113. The intravesical system of any one of embodiments 106-112, wherein the osmotic minitablets comprise a total of: i) about 630 mg to about 670 mg urea; and ii) about 50 mg to about 90 polyethylene oxide (PEO).

114. The intravesical system of embodiment 113, wherein the osmotic minitablets comprise a total of: i) about 648 mg urea; and ii) about 72 mg polyethylene oxide (PEO).

115. The intravesical system of embodiment 113 or 114, wherein the osmotic minitablets further comprise FD&C Blue No. 1 colorant.

116. The intravesical system of any one of embodiments 106-115, wherein the intravesical system is configured as a flexible bi-oval shaped tube.

117. The intravesical system of any one of embodiments 106-116, wherein when in the retention shape, the intravesical system defines a bi-oval coiled shape having a maximum width and a maximum height perpendicular to the maximum width, wherein the maximum width is about 4-7 cm and the maximum height is about 3-6 cm.

118. The intravesical system of embodiment 117, wherein the maximum width is about 5.5 cm and the maximum height is about 4.5 cm.

119. The intravesical system of any one of embodiments 106-118, wherein the intravesical system is sterile.

120. The intravesical system of any one of embodiments 106-119, wherein the intravesical system is configured to freely mobile in a bladder of an individual.

121. The method of any one of embodiments 70-105, wherein such treatment results in a complete response (CR) rate of about 72% to about 90% in a population of patients who have received such treatment; a median duration of response (DoR) of about 9 months to about 30 months in a population of patients who have received such treatment; a 12-month complete response (CR) rate of about 40% to about 50% in a population of patients who have received such treatment; and/or a 12-month duration of response (DoR) rate of about 43% to about 67% in a population of patients who have received such treatment.

122. The method of embodiment 121, wherein such treatment results in a complete response (CR) rate of about 82% in a population of patients who have received such treatment; a median duration of response (DoR) of about 25.8 months in a population of patients who have received such treatment; a 12-month complete response (CR) rate of about 45.9% in a population of patients who have received such treatment; and/or a 12-month duration of response (DoR) rate of about 51% in a population of patients who have received such treatment.

123. A method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a median duration of response (DoR) of about 9 months to about 30 months in a population of patients who have received such treatment.

124. The method of embodiment 123, wherein the median DoR is about 25.8 months.

125. A method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a 12-month complete response (CR) rate of about 40% to about 50% in a population of patients who have received such treatment.

126. The method of embodiment 125, wherein the 12-month CR rate is about 45.9%.

127. A method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a 12-month duration of response (DoR) rate of about 43% to about 67% in a population of patients who have received such treatment.

128. The method of embodiment 127, wherein the 12-month DoR rate is about 51%.

129. A method of treating Bacillus Calmette Guerin (BCG)-unresponsive, non-muscle invasive bladder cancer (NMIBC) with carcinoma in situ (CIS) with or without papillary tumors in an individual comprising inserting an intravesical system comprising about 225 mg of gemcitabine into the bladder of the individual once every three weeks for at least 24 weeks, wherein such treatment results in a median time to onset of response of about 2 months to about 9 months in a population of patients who have received such treatment.

130. The method of embodiment 129, wherein the median time to onset of response is about 2.8 months.

131. The method of any one of embodiments 123-130, further comprising inserting an intravesical system comprising about 225 mg of gemcitabine into the bladder of the individual once every twelve weeks for up to 18 months.

132. A kit comprising an intravesical system and instructions for use to treat Bacillus Calmette Guerin (BCG)-unresponsive, non-muscle invasive bladder cancer (NMIBC) with carcinoma in situ (CIS) with or without papillary tumors, the intravesical system comprising a housing configured for intravesical insertion, the housing defining a drug reservoir lumen and a retention frame lumen, the drug reservoir lumen having a release orifice with an about 150 micron diameter; a retention frame comprising a nitinol wire located in the retention frame lumen; 12 gemcitabine minitablets located within the drug reservoir lumen, the 12 gemcitabine minitablets comprising a total of about 225 mg gemcitabine; 30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets; wherein the gemcitabine minitablets are located closer to the release orifice than the osmotic minitablets; wherein the housing is configured to release gemcitabine from the drug reservoir lumen through the release orifice via osmotic pressure generated by the osmotic minitablets; wherein the housing is elastically deformable between a retention shape configured to retain the system in the individual's bladder and a deployment shape configured for passage of the system through the individual's urethra.

133. The kit of embodiment 132, further comprising a urinary placement catheter adapted for insertion of the intravesical system into the bladder.

134. The kit of embodiment 132 or 133, wherein the intravesical system is sterile.

135. The method of any one of embodiments 70-105 or 121-131, wherein each intravesical system produces a mean urinary excretion on Days 2 through 5 of 21 mg to 32 mg per day as gemcitabine and dFdU.

136. The method of any one of embodiments 70-105 or 121-132, wherein each intravesical system comprises an almost white to light pink-brown colored gemcitabine component at the center surrounded on each side by off white to light blue-colored osmotic components.

137. The intravesical system of any one of embodiments 106-120, comprising an almost white to light pink-brown colored gemcitabine component at the center surrounded on each side by off white to light blue-colored osmotic components.

138. A method of treating bladder cancer in an individual comprising administering to the individual an approved drug product comprising gemcitabine.

139. The method of embodiment 138, wherein the approved drug product is described by the approved drug product label of Example 9.

140. The method of embodiment 138 or 139, wherein such treatment results in a complete response (CR) rate of about 72% to about 90% in a population of patients who have received such treatment; a median duration of response (DoR) of about 9 months to about 30 months in a population of patients who have received such treatment; a 12-month complete response (CR) rate of about 40% to about 50% in a population of patients who have received such treatment; and/or a 12-month duration of response (DoR) rate of about 43% to about 67% in a population of patients who have received such treatment.

141. The method of embodiment 140, wherein such treatment results in a complete response (CR) rate of about 82% in a population of patients who have received such treatment; a median duration of response (DoR) of about 25.8 months in a population of patients who have received such treatment; a 12-month complete response (CR) rate of about 45.9% in a population of patients who have received such treatment; and/or a 12-month duration of response (DoR) rate of about 51% in a population of patients who have received such treatment.

142. A method of improving complete response (CR) rate in a population of individuals, the method comprising administering to the population of individuals an approved drug product comprising gemcitabine.

143. The method of embodiment 142, wherein the CR rate is about 82%.

144. A method of improving median duration of response in a population of individuals, the method comprising administering to the population of individuals an approved drug product comprising gemcitabine.

145. The method of embodiment 144, wherein the median duration of response is about 25.8 months.

146. A method of improving 12-month complete response (CR) rate in a population of individuals, the method comprising administering to the population of individuals an approved drug product comprising gemcitabine.

147. The method of embodiment 146, wherein the 12-month CR rate is about 45.9%.

148. A method of improving 12-month duration of response (DoR) rate in a population of individuals, the method comprising administering to the population of individuals an approved drug product comprising gemcitabine.

149. The method of embodiment 148, wherein the 12-month DoR rate is about 51%.

150. The method of any one of embodiments 138-149, comprising inserting an intravesical system comprising about 225 mg of gemcitabine into the bladder once every 3 weeks up to 6 months (8 doses), followed by once every 12 weeks for up to 18 months (6 doses), or until persistent or recurrent NMIBC, disease progression, or unacceptable toxicity.

151. The method of embodiment 150, wherein each intravesical system is removed after a 3-week indwelling period.

152. A kit comprising: an intravesical device comprising 225 mg gemcitabine, co-packaged with a urinary catheter and stylet, and instructions for use according to any one of embodiments 1-36, 52-63, 70-105, 121-132, or 138-151, or example 10.

153. The method of embodiment 138 or 139, wherein such treatment results in: a) a 24-month duration of response (DoR) rate of about 28% to about 54% in a population of patients who have received such treatment; and/or b) a 24-month cystectomy-free rate of about 61% to about 83% in a population of patients who have received such treatment.

154. The method of embodiment 153, wherein: a) the 24-month DoR rate is about 41.6% in a population of patients who have received such treatment; and/or b) the 24-month cystectomy-free rate is about 73.7% in a population of patients who have received such treatment.

### EXAMPLES

The following examples are exemplary and are not intended to limit the scope of any invention or inventions described herein.

### Example 1: In Vitro Release Rate of Gemcitabine Following Administration of TAR-200

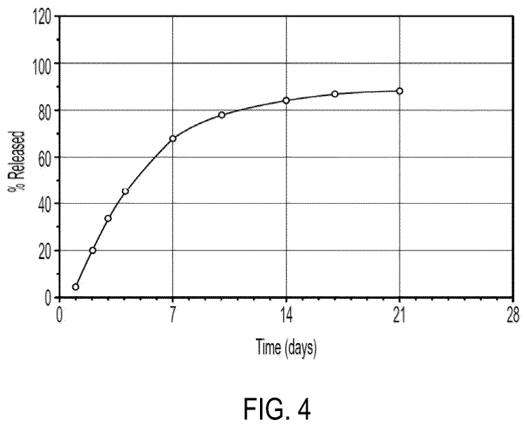

FIG. 4 depicts the percent of gemcitabine released from the TAR-200 intravesical system in vitro. The intravesical system containing 225 mg gemcitabine (256 gemcitabine HCl) was placed in 300 mL ultrapure water at 37 degrees C. 1 mL samples were taken at 1, 2, 3, 4, 7, 10, 14, 17, and 21 days after the system was placed in water. Samples were then analyzed using an Acquity UPLC® with a Charged Surface Hybrid C18 column (1.7-μm particle size, 50×2.1 mm, Waters Corporation, Milford, MA) to quantify gemcitabine released from the intravesical system. The results show that approximately 70% of the gemcitabine was released from the intravesical system by day 7, and that approximately 90% of the gemcitabine was released by day 21.

### Example 2: Bladder Concentrations of Gemcitabine in Minipigs Following Administration of TAR-200

Nonclinical studies have demonstrated that bladder concentrations of gemcitabine following administration of TAR-200 in minipigs are sufficient to achieve tissue concentrations that are expected to be tumoricidal.

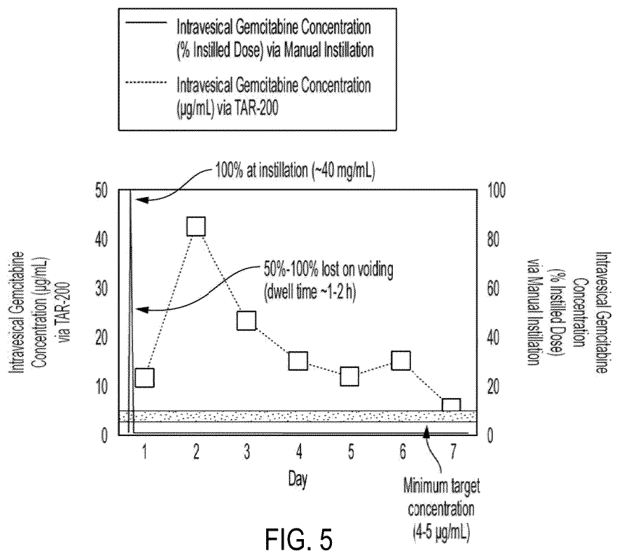

A non-Good Laboratory Practice (non-GLP) toxicology study of TAR-200 in minipigs demonstrated that average bladder urine concentrations of 10 μg/mL and above were achieved over a 7-day dosing cycle (FIG. 5) resulting in no or low plasma levels of gemcitabine. In this study, minimal changes in hematological tests, clinical chemistry tests, and urinalyses were noted in animals treated with 1 deployment of TAR-200 or 2 sequential 7-day deployments of TAR-200. Administration of 2 TAR-200 simultaneously in this study resulted in greater urinary concentrations and more pronounced clinical and histopathological findings.

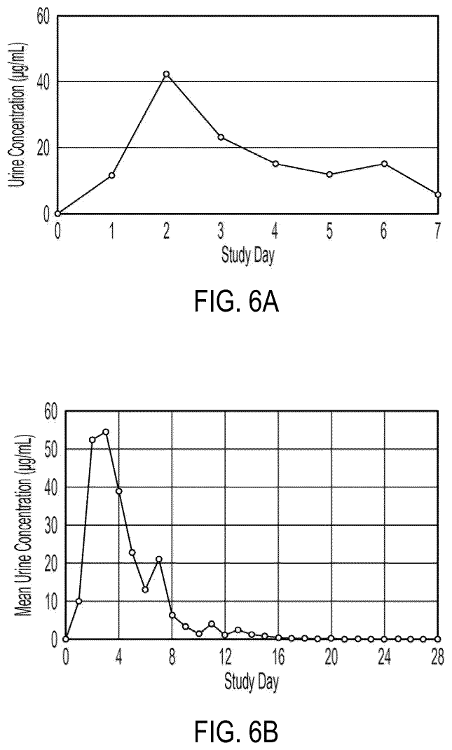

A GLP toxicology study comparing TAR-200 to placebo demonstrated that quantifiable urine gemcitabine concentrations occurred within 24 hours and levels at or above 10 μg/mL were generally maintained over a 7-day dosing cycle in minipigs (FIG. 6A). No quantifiable levels of gemcitabine were observed in plasma. Administration of TAR-200 was associated primarily with mild urothelial inflammation in the bladder based on the hematology, clinical chemistry, gross pathology, and microscopic observations. The urothelial changes tended to be mild in severity and transient as complete resolution was observed following the 14-day recovery period.

Use of TAR-200 for up to 21 days is based on the biphasic release properties observed in vitro and confirmed by in vivo minipig studies. In minipigs, during the first 7-day period, approximately 70% to 80% of the total gemcitabine content in TAR-200 is released, with <20% being released over the following 14-day period (FIG. 6B).

### Example 3: Phase 2b Clinical Study Evaluating Efficacy and Safety of TAR-200 Monotherapy in Participants with BCG-Unresponsive, HR-NMIBC with CIS, with or without Papillary Tumors

This study was an open-label, parallel-group, multi-center study evaluating the efficacy and safety of intravesical TAR-200 monotherapy, systemic intravenous (IV) cetrelimab monotherapy, or intravesical TAR-200 in combination with systemic IV cetrelimab in participants with HR NMIBC (CIS with or without papillary disease) unresponsive to prior intravesical BCG therapy, who are either ineligible for or have elected not to undergo radical cystectomy (RC) after making an informed decision. Participants were randomly assigned (1:1:2) to receive intravesical TAR-200 in combination with systemic IV cetrelimab, intravesical TAR-200 monotherapy, or IV cetrelimab monotherapy.

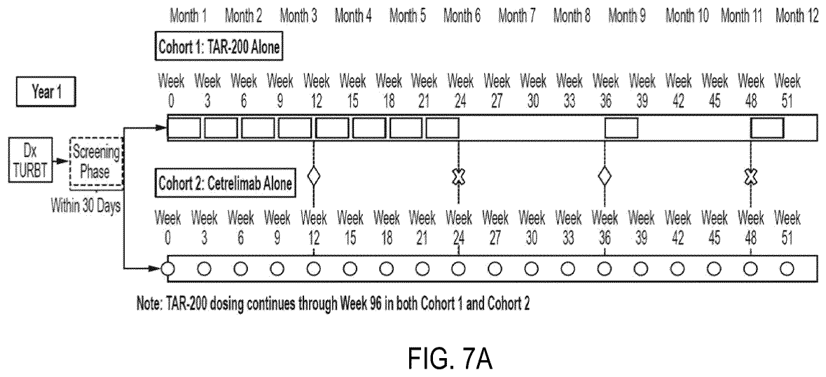

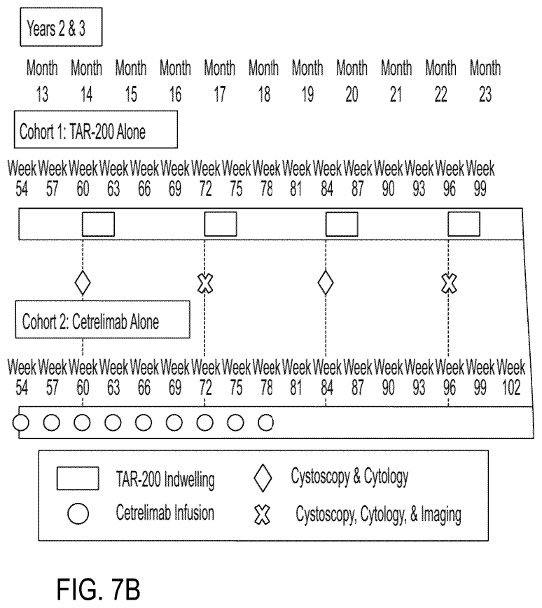

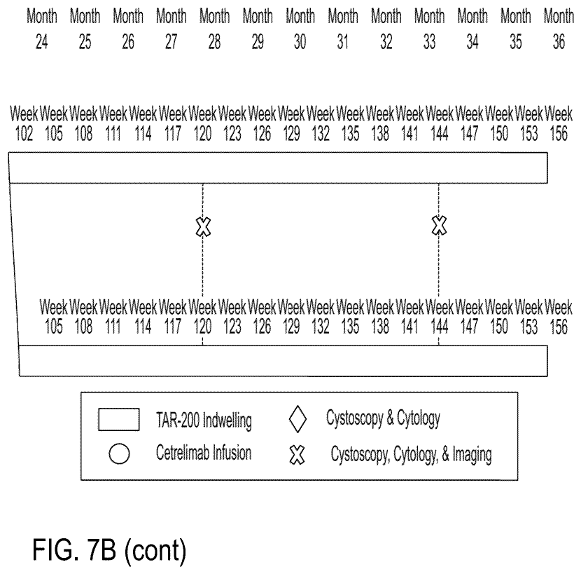

For TAR-200 monotherapy, TAR-200 was dosed every 3 weeks (Q3W) for up to the first 24 weeks (6 months) on study, as shown in FIG. 7A. Dosing of TAR-200 then occurred every 12 weeks through Week 99 (Year 2, as shown in FIG. 7B). Each TAR-200 was removed after a 3-week dwell time through Week 99 (Year 2). For cetrelimab monotherapy, cetrelimab was dosed at 360 mg IV Q3W through Week 78 (18 months), as shown in FIGS. 7A and 7B. For intravesical TAR-200 in combination with systemic IV cetrelimab, TAR-200 was dosed Q3W for up to the first 24 weeks (6 months), as shown in FIG. 7A. Dosing of TAR-200 then occurred every 12 weeks through Week 99 (Year 2, as shown in FIG. 7B). Each TAR-200 was removed after a 3-week dwell time through Week 99 (Year 2). Cetrelimab was dosed at 360 mg IV Q3W through Week 78 (18 months). Dosing of TAR-200 then occurred every 12 weeks through Week 99 (Year 2).

### Study Primary Objective

The primary study objective was to evaluate the complete response (CR) rate in participants treated with TAR-200 monotherapy or IV cetrelimab monotherapy with Carcinoma in Situ (CIS), with or without concomitant high-grade Ta or T1 papillary disease.

### Study Primary Endpoint

The primary efficacy endpoint was complete response rate, defined as the percentage of patients achieving a complete response at any time post-treatment. Complete response was defined as at least one of the following: (1) negative cystoscopy and negative (including atypical) urine cytology, or (2) positive cystoscopy with biopsy-proven benign or low-grade NMIBC and negative (including atypical) urine cytology. For example, the 12-month CR rate is defined as the proportion of patients who have achieved a CR that lasts for at least 12 months.

### Study Secondary Objectives

The secondary objectives of this study included the following. 1) To evaluate the duration of response (DOR) in participants treated with TAR-200 monotherapy or IV cetrelimab monotherapy with CIS (with or without concomitant Ta or T1 papillary disease) who achieve a CR. 2) To determine the overall survival (OS) in all participants, defined as the time from the date of first dose of study treatment to death; if a participant has not died at the time of analysis, the participant will be censored at the date last known alive. 3) To evaluate the pharmacokinetics (PK) of gemcitabine and major metabolite 2′,2′ difluorodeoxyuridine (dFdU) in urine and plasma in TAR-200 monotherapy. 4) To evaluate the PK and immunogenicity of IV cetrelimab in serum in cetrelimab monotherapy. 5) To evaluate health-related quality of life (HRQOL) in all participants. 6) To assess the safety and tolerability of participants receiving TAR-200 monotherapy or IV cetrelimab alone monotherapy.

DOR rate is defined as the proportion of patients who have achieved a DOR that lasts for at least a set time period. For example, the 12-month DOR rate is defined as the proportion of patients who have achieved a DOR that lasts for at least 12 months.

Response was determined by quarterly cystoscopy, quarterly central cytology, mandated bladder biopsy by central assessment at Weeks 24 and 48, and local imaging Q24W. The study protocol did not allow re-induction for nonresponders, consistent with US FDA guidance.

Safety analyses were performed on the treated analysis set defined as all randomized participants who received at least one dose of any study intervention. Summaries of treatment-emergent adverse events (TEAEs), serious TEAEs, TEAEs by toxicity grade, study drug/device/procedure related TEAEs, and TEAEs leading to treatment discontinuation or death, if any, are provided.

Patient characteristics, safety results, and efficacy results for the TAR-200 monotherapy arm are provided below in Table 1.

TABLE 1

| | TAR-200 Monotherapy | |
|---|---|---|
| Characteristics | (N = 85) | |
| Age, years, median (range) | 71 | (40-88) |
| Sex, male, % | 80.0 | |
| Race, % | | |
| White | 87.1 | |
| Asian | 9.4 | |
| Black or African American | 2.4 | |
| Not reported/unknown | 1.2 | |
| Nicotine use, % | | |
| Current | 8.2 | |
| Former | 58.8 | |
| Never | 32.9 | |
| ECOG PS 0, % | 91.8 | |
| Tumor stage, % | | |
| CIS only | 67.1 | |
| CIS + papillary disease | 32.9 | |
| CIS + Ta | 22.4 | |
| CIS + T1 | 10.6 | |
| Total doses of prior BCG, n, median (range) | 12 | (7-42) |
| Time from last BCG to CIS diagnosis, months, | 3.2 | (0-22) |
| median (range) | | |
| Reason for not receiving RC, % | | |

| | |
|---|---|
| Declined | 96.5 |
| Ineligible | 3.5 |

### Results

At a clinical cutoff of Sep. 4, 2024, 85 patients were assigned to TAR-200 monotherapy; nine (10.6%) had completed study treatment and 68 (80.0%) were still on study.

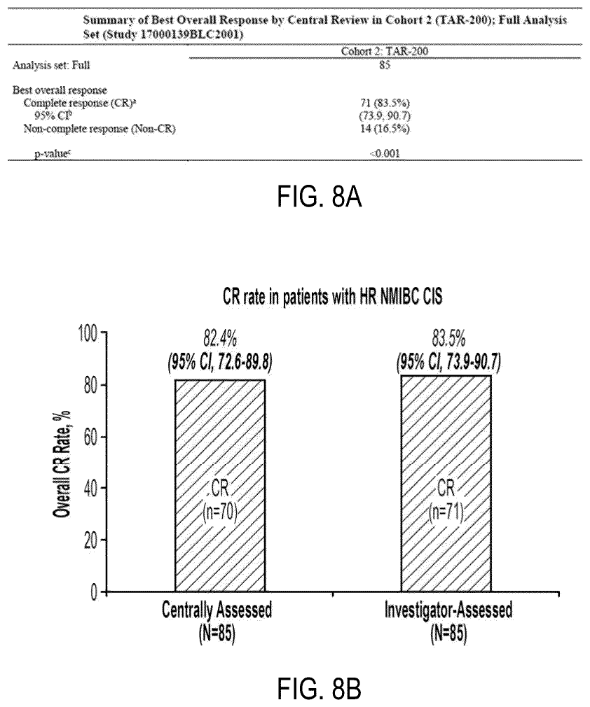

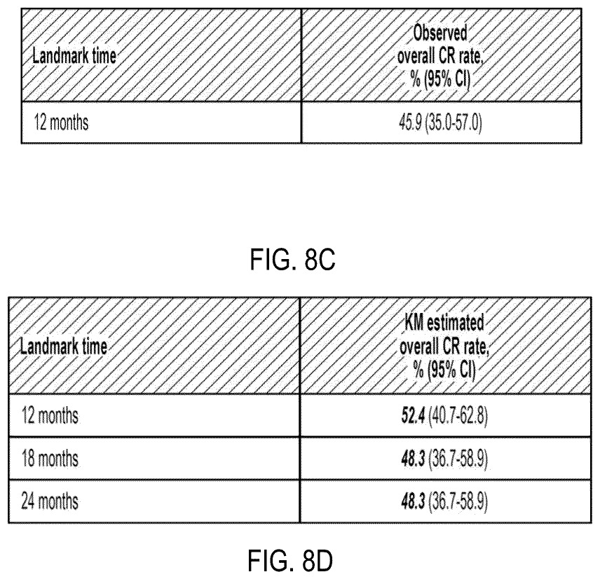

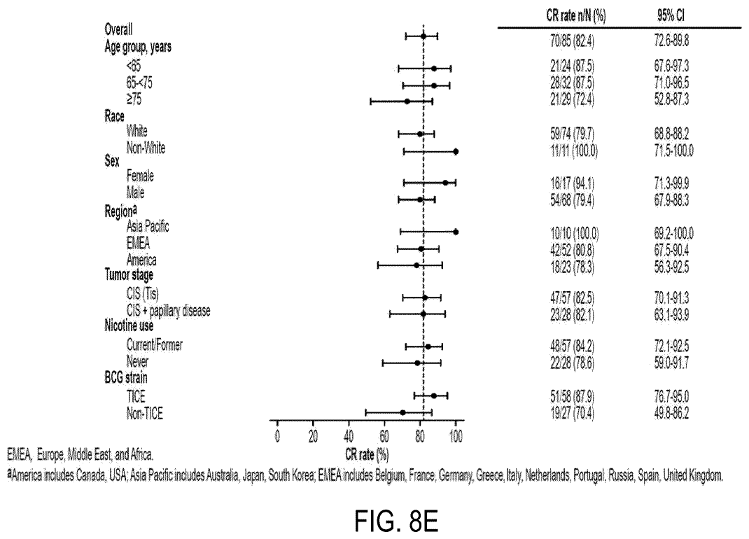

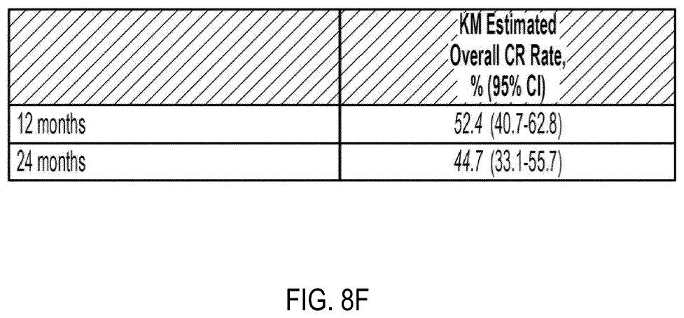

Of 85 patients, 71 (83.5% [95% CI, 73.9 to 90.7]) had a centrally confirmed complete response (FIG. 8A). Investigator-assessed complete response rate was 83.5% (95% CI, 73.9 to 90.7), and the overall concordance rate was 93.8%. Median time to response was 2.8 months (range, 1.4 to 8.3). Landmark complete response rates at 12 and 18 months were 32.9% (95% CI, 23.1-44.0) and 15.3% (95% CI, 8.4-24.7), respectively (FIGS. 8C and 8D).

Median duration of response was 25.8 months (95% CI, 8.6 to NE). At clinical cutoff, 38 responses (53.5%) were ongoing with no event after median follow-up of 13.9 months (range, 5 to 41). Overall survival rates at 6- and 12-months were 98.7% (95% CI, 91.2 to 99.8) and 93.4% (95% CI, 83.2 to 97.5), respectively.

Mean global health status (75 [standard deviation [SD], 16.7]) and physical functioning (86 [SD, 17.3]) scores were high at baseline and LS mean changes from baseline did not exceed the clinically meaningful threshold (≥10 points) during treatment.

Median duration of TAR-200 exposure was 38.9 weeks (range, 1 to 108), with median of 8 (1 to 14) TAR-200 insertions and median indwelling duration of 22 days (5 to 25).

Treatment-related adverse events of any grade occurred in 71 patients (83.5%). Adverse events resolved after a median of 2.9 weeks (range, 0.1 to 137.3). The most frequent treatment-related adverse events were primarily low-grade lower urinary tract events, including pollakiuria (40.0%), dysuria (36.5%), micturition urgency (21.2%), and urinary tract infection (21.2%). Grade ≥3 treatment-related adverse events were reported in 10 patients (11.8%) and included urinary tract pain (4.7%), acute kidney injury, bladder pain, cystitis, urinary retention, urinary tract disorder, urinary tract infection, and urosepsis (in one patient each). Treatment-related serious adverse events occurred in five patients (5.9%); grade ≥3 treatment-related serious adverse events included acute kidney injury, urinary tract infection, urinary tract pain, and urosepsis, reported in one patient each. Frequent concomitant medications used to manage adverse events included antibacterial (97.6%) and urological (69.4%) agents.

Treatment-related adverse events leading to TAR-200 interruption occurred in 27 patients (31.8%). Three patients (3.5%) discontinued treatment due to treatment-related adverse events, including 2 (2.4%) with noninfective cystitis, 1 (1.2%) with pollakiuria, and 1 (1.2%) with urinary tract disorder.

No treatment-related adverse events leading to death were reported among patients who received TAR-200 monotherapy, however, five deaths unrelated to treatment occurred (adverse events [cognitive disorder and sepsis], surgical complications, and progressive disease).

At a clinical cutoff of Feb. 18, 2025, 85 patients with BCG-unresponsive carcinoma in situ were enrolled in the TAR-200 monotherapy cohort. Median age was 71 years; most patients were male (80.0%) and white (87.1%) (Table 1). One third of patients (32.9%) had concurrent papillary disease (high-grade Ta or T1). Patients had median 12 prior BCG doses, and most refused radical cystectomy (96.5%).

The centrally confirmed complete response rate was 82.4% (95% CI, 72.6 to 89.8), with 70 of 85 patients achieving complete response (FIG. 8B). Investigator-assessed complete response rate was 83.5% (95% CI, 73.9 to 90.7), and the overall concordance between central and investigator-assessed complete response rate was 95.0%. Ninety-six percent of responses were achieved at first disease evaluation (median time to response, 2.8 months). At 3-, 6-, and 12-month response assessments, complete response rates were 78.8% (95% CI, 68.6 to 86.9), 58.8% (95% CI, 47.6 to 69.4), and 45.9% (95% CI, 35.0 to 57.0). Subgroup analyses showed a consistently high complete response rate across all clinically relevant subgroups, including patients with and without concurrent papillary disease (82.1% and 82.5%, respectively) (FIG. 8E).

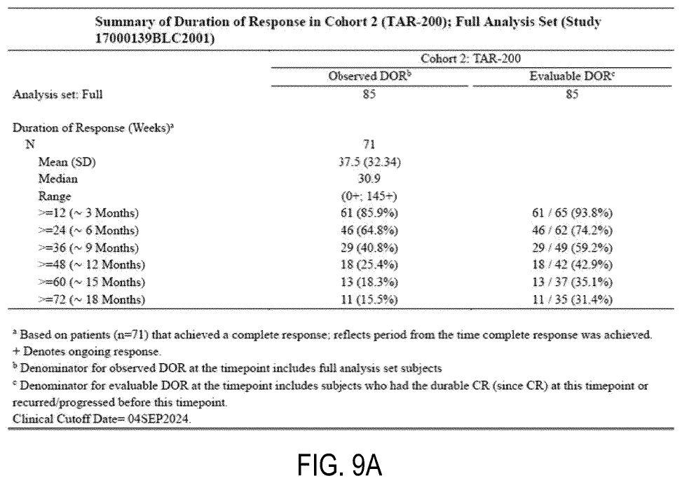

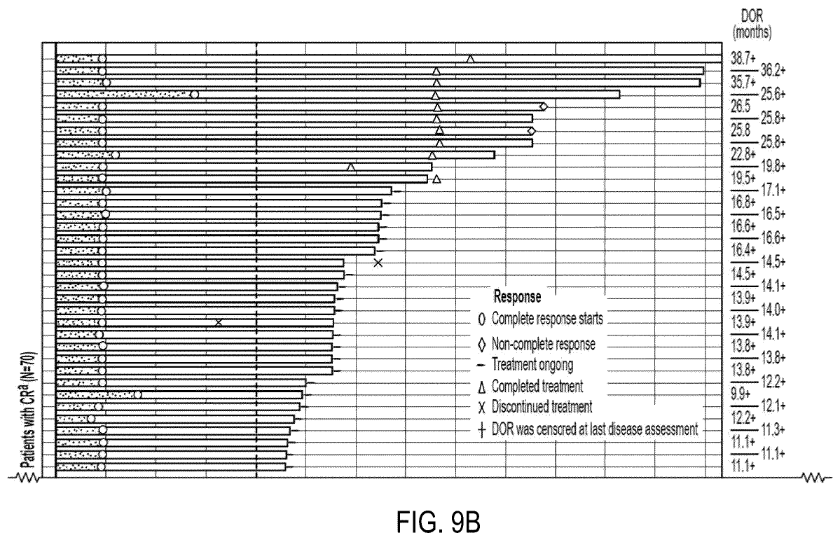

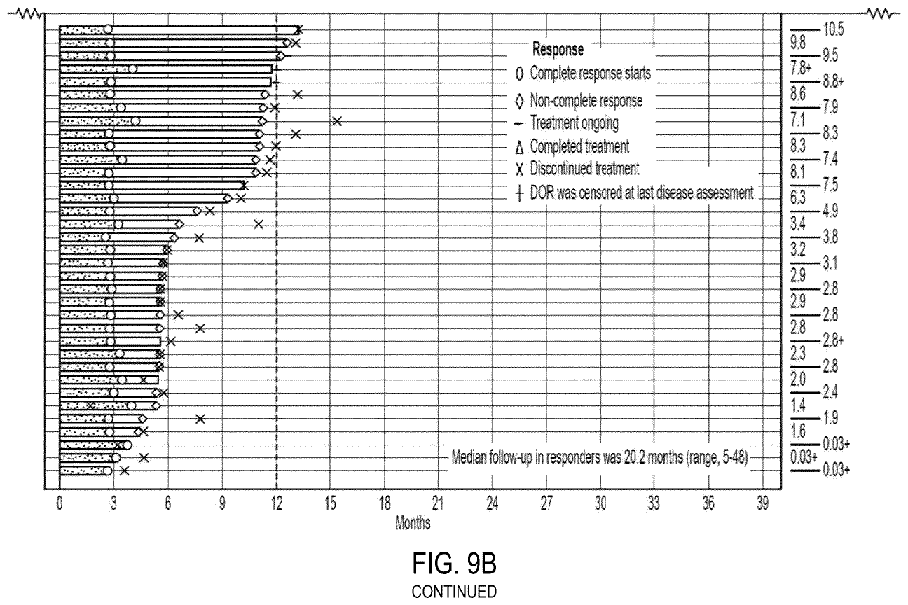

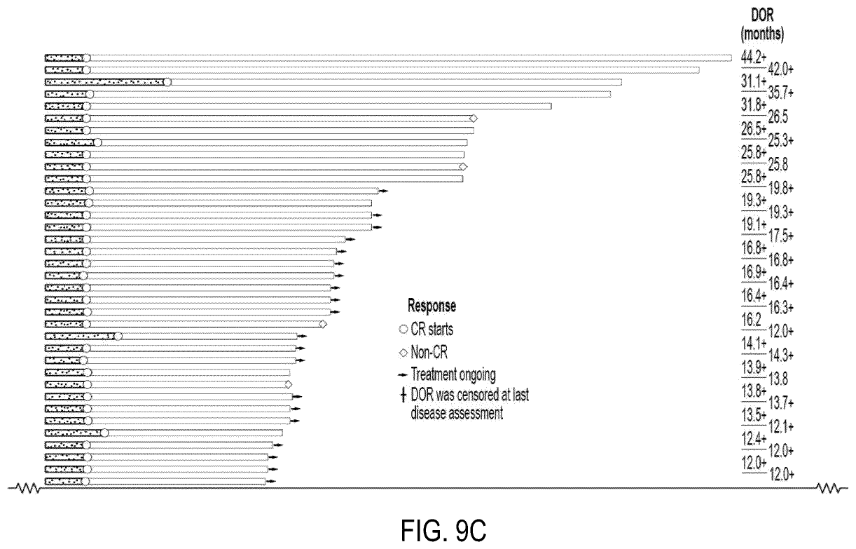

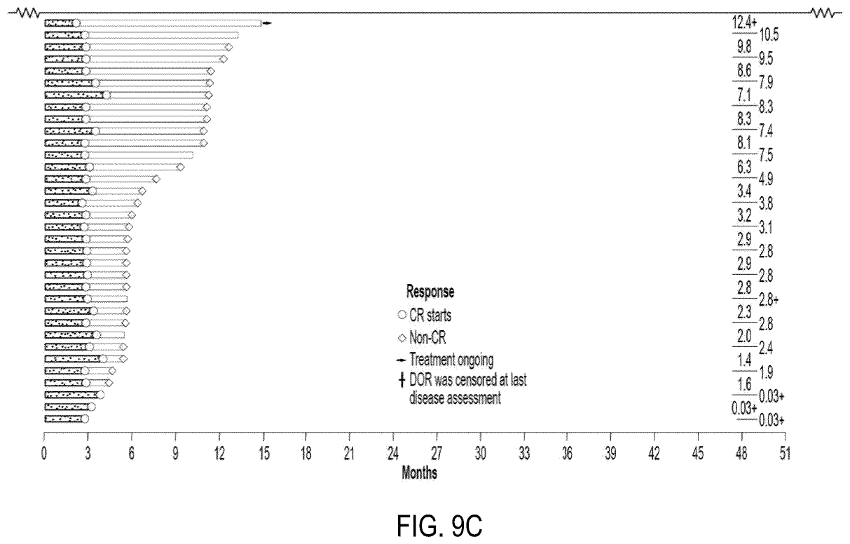

After median follow-up in responders of 20.2 months (range, 5 to 48), median duration of response was 25.8 months (95% CI, 8.3 to NE) (FIG. 9B). Twelve-month duration of response rate was 55.7% (95% CI, 42.9 to 66.8). Thirty-nine of 70 responders remained in complete response at clinical cutoff, with 11 patients having completed 2 years of treatment. Among responders, 28 (40.0%) had recurrence, with 19 with high-risk NMIBC only, two progressed to MIBC (both T2), one to lymph node positive disease, and one to metastasis. Moreover, 25 patients received subsequent treatment, including 17 patients (20.0%) who underwent radical cystectomy. The median time to cystectomy was not estimable. The 12-month radical cystectomy-free rate was 86.6% (95% CI, 76.5 to 92.6).

Overall survival rates at 6 and 12 months were 98.7% (95% CI, 91.2 to 99.8) and 94.7% (95% CI, 86.5 to 98.0), respectively, with a total of 6 deaths (7.1% of 85 patients). No deaths were treatment-related.

Treatment-related adverse events are summarized in Table 2 below. Treatment-related adverse events of any grade occurred in 71 patients (83.5%). The most frequent treatment-related adverse events were low-grade lower urinary tract events, including pollakiuria (40.0%), dysuria (37.6%), micturition urgency (22.4%), and urinary tract infection (22.4%). Adverse events resolved after a median of 3.1 weeks (range, 0.1+ to 167.0+). Grade ≥3 treatment-related adverse events were reported in 12 patients (14.1%), with urinary tract pain most frequent (4.7%). Treatment-related serious adverse events occurred in five patients (5.9%), with cystitis with bladder pain (grade 2), pseudomonal cystitis (grade 2), urinary tract infection (grade 3), urosepsis with acute kidney injury (grade 3), and urinary tract pain (grade 3) occurring in 1 patient each.

Treatment-related adverse events leading to TAR-200 interruption occurred in 27 patients (31.8%), with urinary tract pain (5.9%), urinary tract infection (4.7%), and pollakiuria (4.7%) most frequent. Most interruptions were limited to 1-2 doses, and most patients resumed treatment. Four patients (4.7%) discontinued TAR-200 due to treatment-related adverse events, including noninfective cystitis (2 patients), pollakiuria and urinary tract disorder (“other [burning]”) (1 patient), and anal incontinence (1 patient).

TABLE 2

| | TAR-200 Monotherapy | | |
|---|---|---|---|
| | (N = 85) | | |
| Adverse events | Any grade | | Grade ≥3 |
| ≥1 Treatment-related adverse events | 71 | (83.5) | 12 (14.1) |
| Most frequent treatment-related adverse | | | |
| events | | | |
| Pollakiuria | 34 | (40.0) | 0 |
| Dysuria | 32 | (37.6) | 0 |
| Micturition urgency | 19 | (22.4) | 0 |
| Urinary tract infection | 19 | (22.4) | 1 (1.2) |
| Hematuria | 14 | (16.5) | 0 |
| Urinary tract pain | 9 | (10.6) | 4 (4.7) |
| Bladder pain | 6 | (7.1) | 1 (1.2) |
| Noninfective cystitis | 6 | (7.1) | 0 |
| Bladder spasm | 5 | (5.9) | 0 |
| Lower urinary tract symptoms | 5 | (5.9) | 0 |
| Urinary incontinence | 5 | (5.9) | 1 (1.2) |
| Nocturia | 4 | (4.7) | 0 |
| Urethral pain | 4 | (4.7) | 0 |
| Urinary retention | 4 | (4.7) | 1 (1.2) |
| Constipation | 3 | (3.5) | 0 |
| Cystitis | 3 | (3.5) | 1 (1.2) |
| Pelvic pain | 3 | (3.5) | 0 |
| Abdominal pain | 2 | (2.4) | 0 |
| Abdominal pain lower | 2 | (2.4) | 0 |
| Asthenia | 2 | (2.4) | 0 |
| Fatigue | 2 | (2.4) | 0 |
| Penile pain | 2 | (2.4) | 0 |
| Perineal pain | 2 | (2.4) | 0 |
| Urethral injury | 2 | (2.4) | 0 |
| Vulvovaginal pain | 2 | (2.4) | 0 |
| Treatment-related adverse events leading to | 27 (31.8%) | | |
| TAR-200 interruption | | | |
| Treatment-related adverse events leading to | 4 (4.7%) | | |
| TAR-200 discontinuation | | | |

Mean global health status (75.0 [standard deviation [SD], 16.7]) and physical functioning (86.2 [SD, 17.3]) scores were high at baseline and were maintained during treatment (did not exceed clinically meaningful change threshold of ≥10 points).

### Conclusion

These results establish TAR-200 monotherapy as the first intravesical drug-releasing system with demonstrated efficacy and safety in the treatment of localized bladder cancer. TAR-200 monotherapy provides sustained localized delivery of intravesical gemcitabine with a favorable benefit-risk profile, supporting TAR-200 as a novel bladder-sparing treatment option for patients with BCG-unresponsive high-risk NMIBC.

### Example 4: Urine Concentration-Time Profile of Gemcitabine in Individuals Following Administration of TAR-200

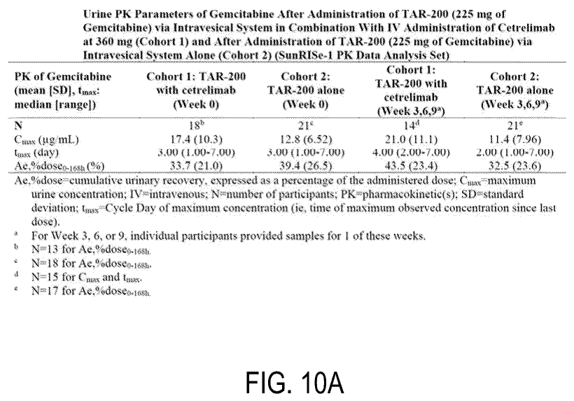

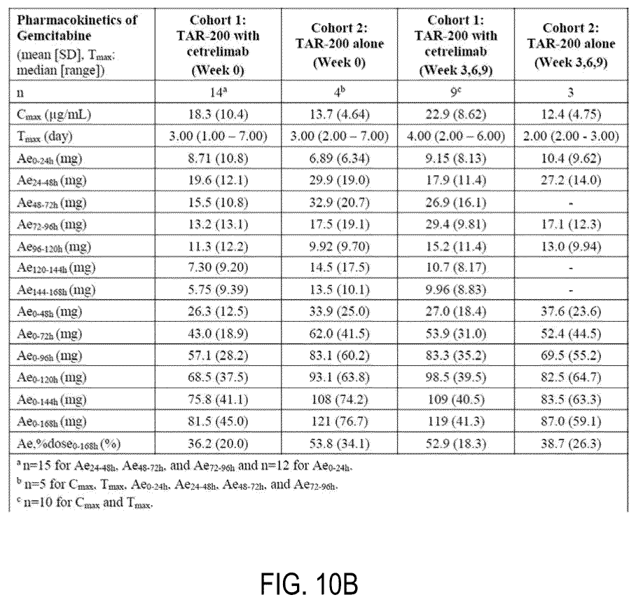

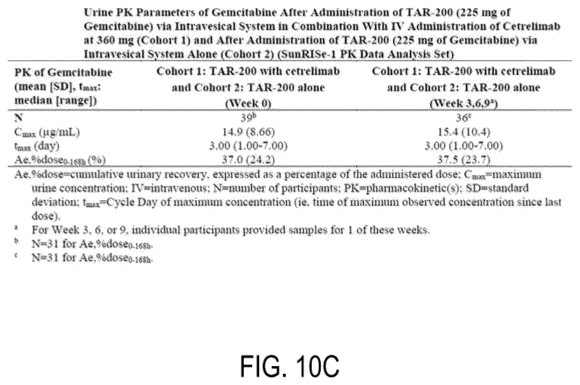

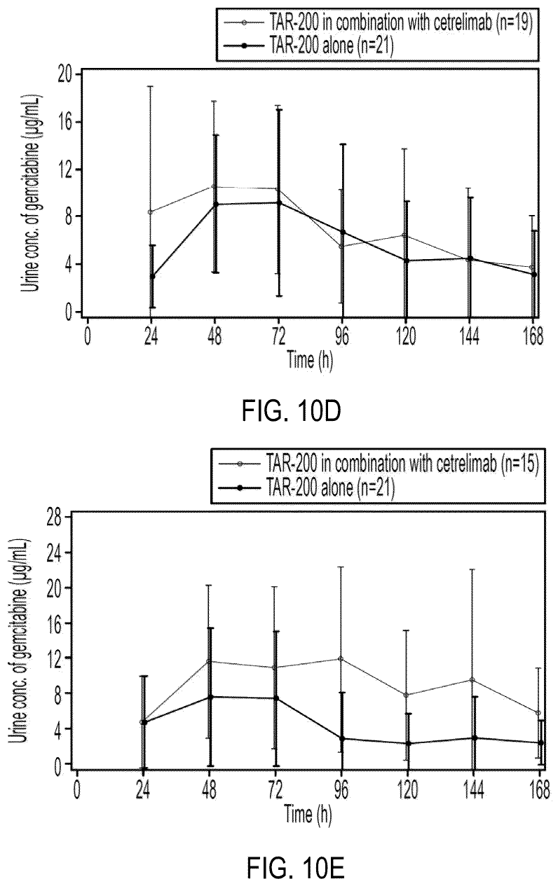

Pharmacokinetics of gemcitabine in urine were measured in individuals having non-muscle invasive bladder cancer (NMIBC) and receiving TAR-200 (225 mg gemcitabine; 256 mg gemcitabine HCL) or TAR-200 in combination with intravenous cetrelimab (360 mg) for three-week dosing cycles of gemcitabine. After administration of gemcitabine via the TAR-200 intravesical system, maximum gemcitabine concentration in urine was reached at a median Tₘₐₓ of 2 to 4 days and appeared to be similar for TAR-200+cetrelimab and TAR-200 monotherapy. Mean gemcitabine concentration in urine exceeded the tumoricidal concentrations of 3 μg/mL (FIGS. 10A-10E). Mean cumulative excreted gemcitabine in urine expressed as percentage of the administered dose from 0-168 hours ranged between 36.2-53.8%. Mean cumulative excreted gemcitabine amount in urine expressed as percentage of the administered dose from 0 to 168 hours was 37.0% during the first dose. Mean urine Cₘₐₓ of gemcitabine was 14.9 μg/mL (CV % 58.1%) with a median tₘₐₓ of 3 days.

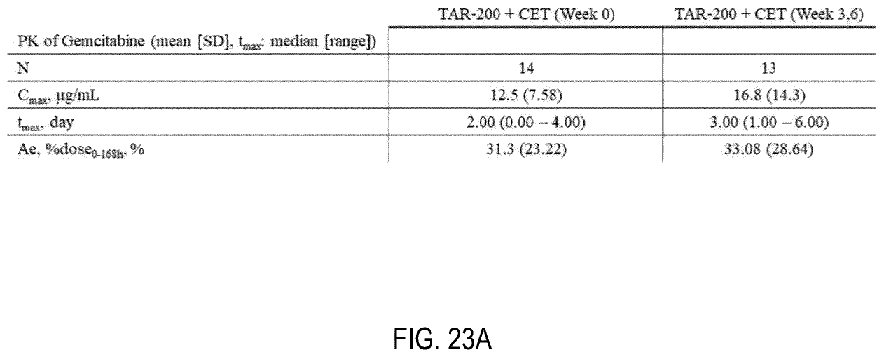

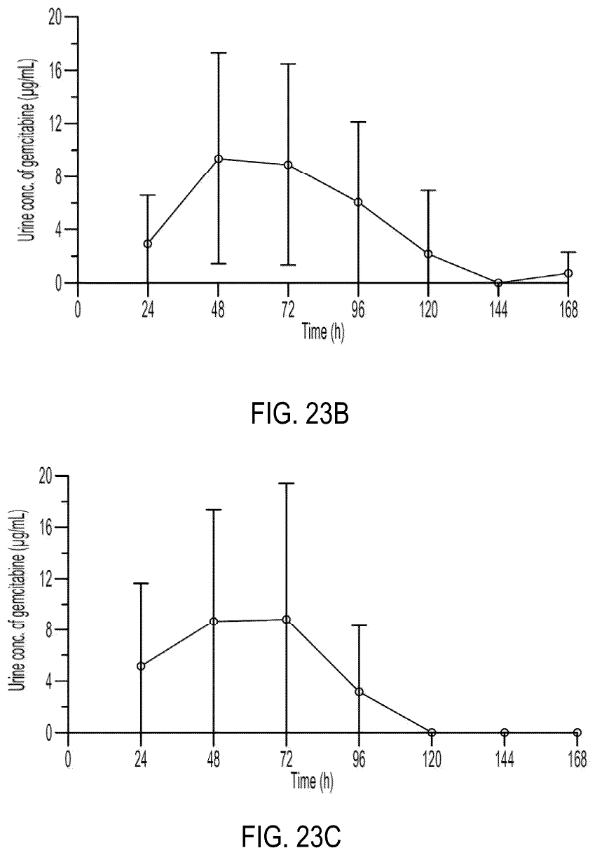

Additionally, pharmacokinetics of gemcitabine in urine were measured in individuals having muscle invasive bladder cancer (MIBC) and receiving TAR-200 (225 mg gemcitabine; 256 mg gemcitabine HCL) in combination with intravenous cetrelimab (360 mg) for three-week dosing cycles of gemcitabine. After administration of gemcitabine via the TAR-200 intravesical system, maximum gemcitabine concentration in urine was reached at a median Tₘₐₓ of 2 to 3 days. Mean gemcitabine concentration in urine exceeded the tumoricidal concentrations of 3 μg/mL (FIGS. 23A-23C). Mean cumulative excreted gemcitabine amount in urine expressed as percentage of the administered dose from 0 to 168 hours was approximately 31.3% during the first dose. Mean urine Cₘₐₓ of gemcitabine was 12.5 μg/mL (CV % 60.6%) with a median tₘₐₓ of 2 days.

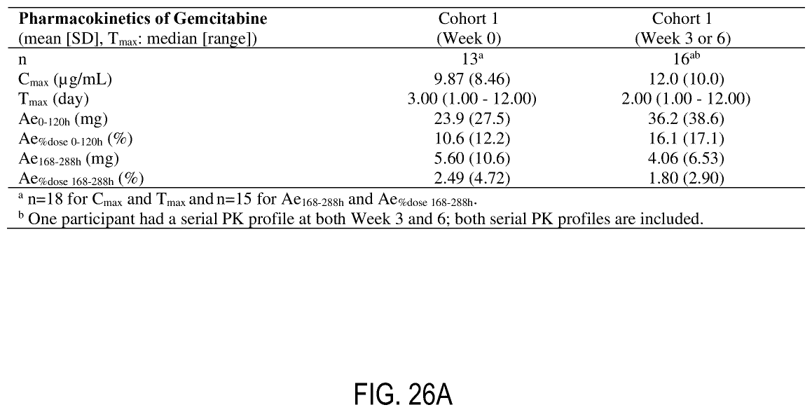

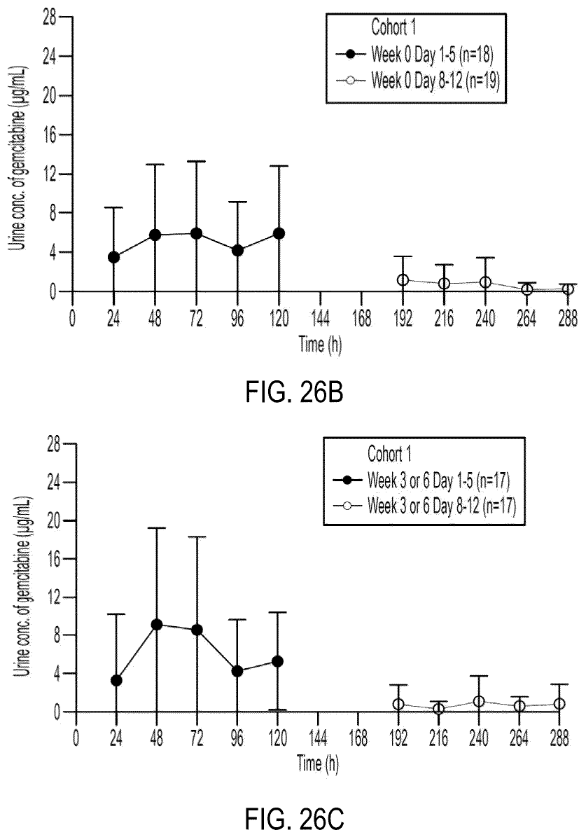

Pharmacokinetics of gemcitabine in urine were also measured in individuals having muscle invasive bladder cancer (MIBC) and receiving TAR-200 (225 mg gemcitabine; 256 mg gemcitabine HCL) in combination with intravenous cetrelimab (360 mg) for three-week dosing cycles of gemcitabine prior to radical cystectomy. After administration of gemcitabine via the TAR-200 intravesical system, maximum gemcitabine concentration in urine was reached at a median Tₘₐₓ of 3 days. Mean gemcitabine concentration in urine exceeded the tumoricidal concentrations of 3 μg/mL (FIGS. 26A-26C). Mean cumulative excreted gemcitabine amount in urine expressed as percentage of the administered dose was approximately 10.6% from 0 to 120 hours (Day 1 to Day 5 since the last dose) and 2.49% from 168 to 288 hours (Day 8 to Day 12 since the last dose). Mean urine Cₘₐₓ of gemcitabine was 9.87 μg/mL (CV % 85.8%) with a median tₘₐₓ of 3 days.

### Example 5: Urine Concentration-Time Profile of dFdU in Individuals Following Administration of TAR-200

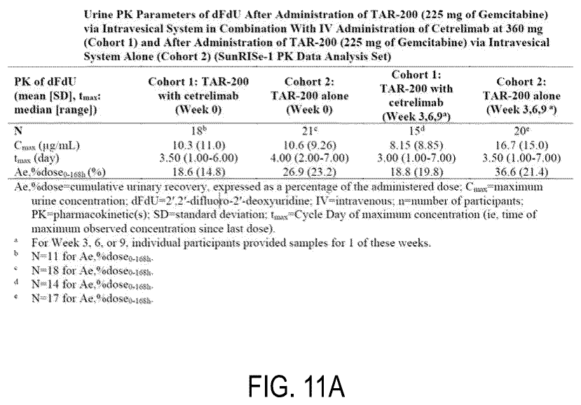

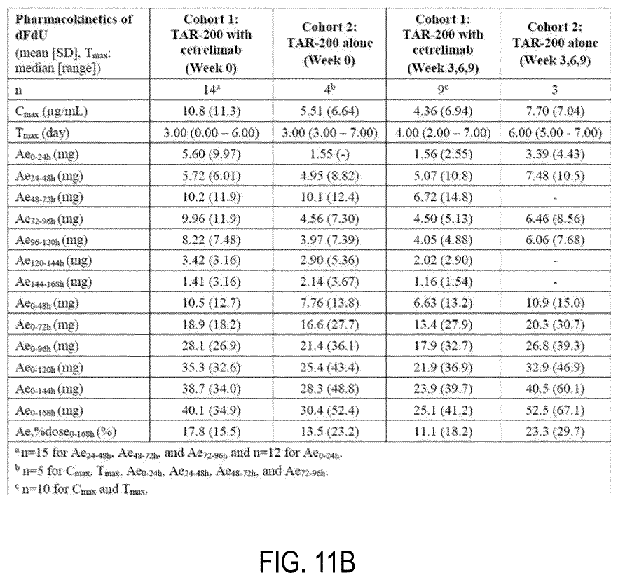

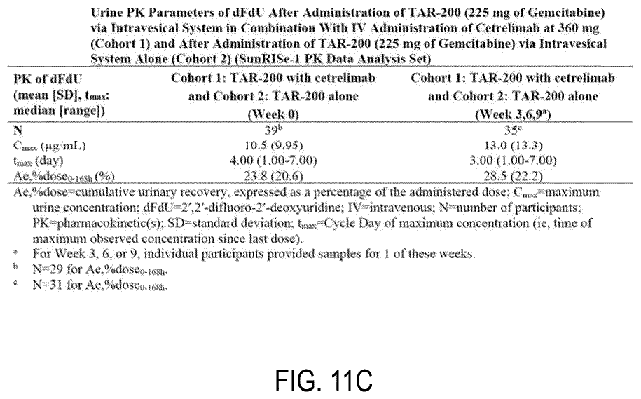

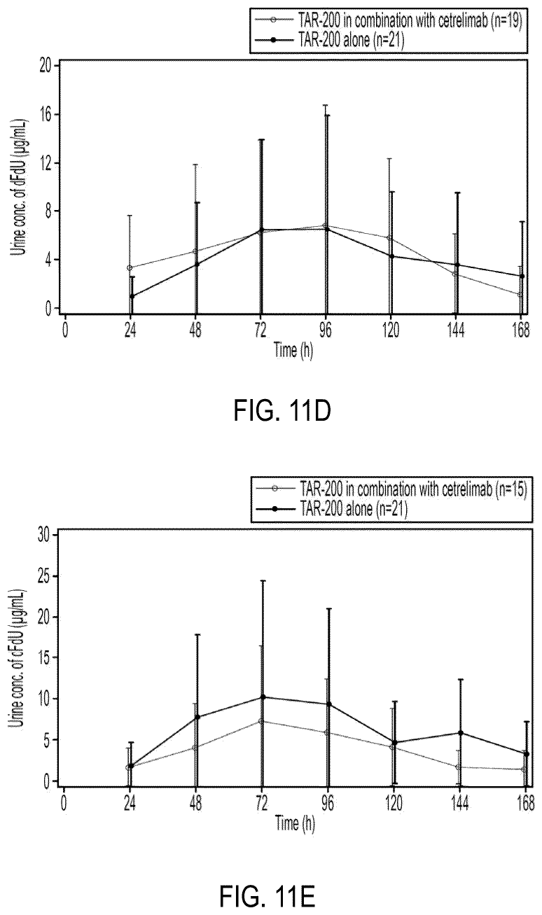

dFdU is an inactive metabolite, generated by deamination of gemcitabine. Pharmacokinetics of dFdU in urine were measured in individuals having NMIBC and receiving TAR-200 (225 mg gemcitabine; 256 mg gemcitabine HCL) or TAR-200 in combination with intravenous cetrelimab (360 mg) for three-week dosing cycles of gemcitabine. Maximum dFdU concentration in urine was reached at median Tₘₐₓ between 3 and 6 days. Maximum mean dFdU concentration in urine for Cohort 1 at Week 3, 6, 9 and Cohort 2 at Week 0, appeared to be comparable but slightly lower than the other weeks. Mean cumulative excreted dFdU amount in urine expressed as percentage of the administered dose from 0-168 hours ranged between 11.1-23.3%. (FIGS. 11A-11E). Mean urine Cₘₐₓ of dFdU was 10.5 μg/mL (CV % 94.8%) with a median tₘₐₓ of 4 days. Mean cumulative excreted dFdU amount in urine expressed as percentage of the administered dose from 0 to 168 hours was 23.8% for the dose administered on Week 0.

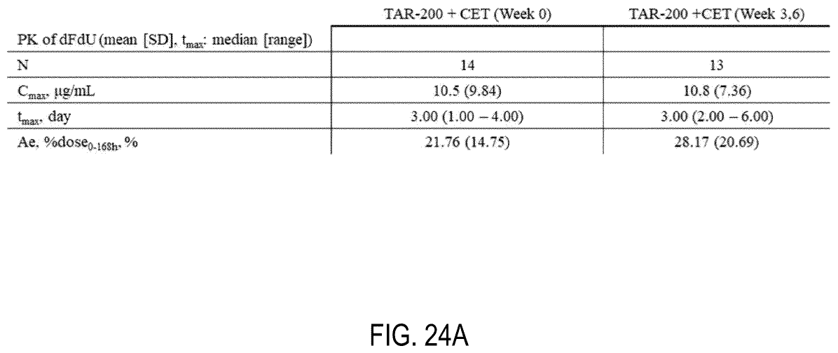

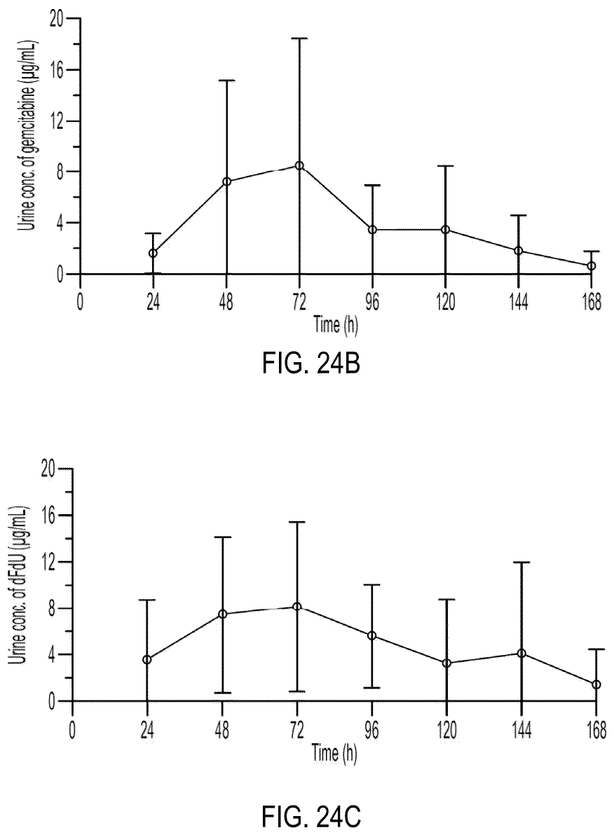

Additionally, pharmacokinetics of dFdU in urine were measured in individuals having MIBC and receiving TAR-200 (225 mg gemcitabine; 256 mg gemcitabine HCL) in combination with intravenous cetrelimab (360 mg) for three-week dosing cycles of gemcitabine. Maximum dFdU concentration in urine was reached at median Tₘₐₓ of 3 days. Mean cumulative excreted dFdU amount in urine expressed as percentage of the administered dose from 0-168 hours ranged between 21.76-28.17%. (FIGS. 24A-24C). Mean urine Cₘₐₓ of dFdU was between 10.5-10.8 μg/mL with a median tₘₐₓ of 3 days. Mean cumulative excreted dFdU amount in urine expressed as percentage of the administered dose from 0 to 168 hours was about 21.76% for the dose administered on Week 0.

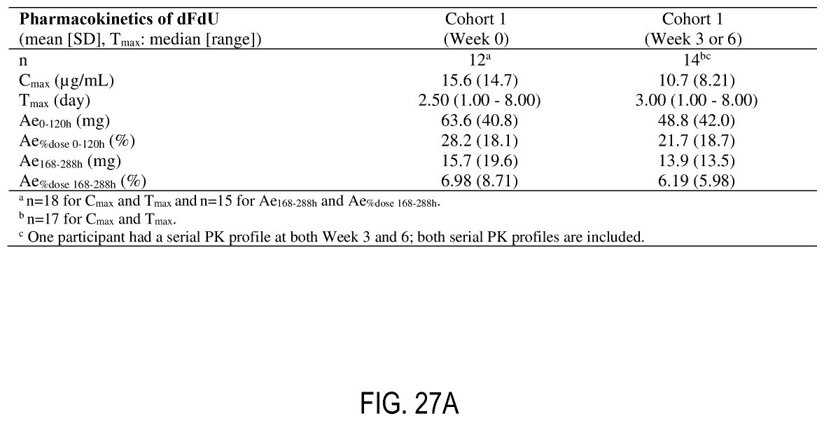

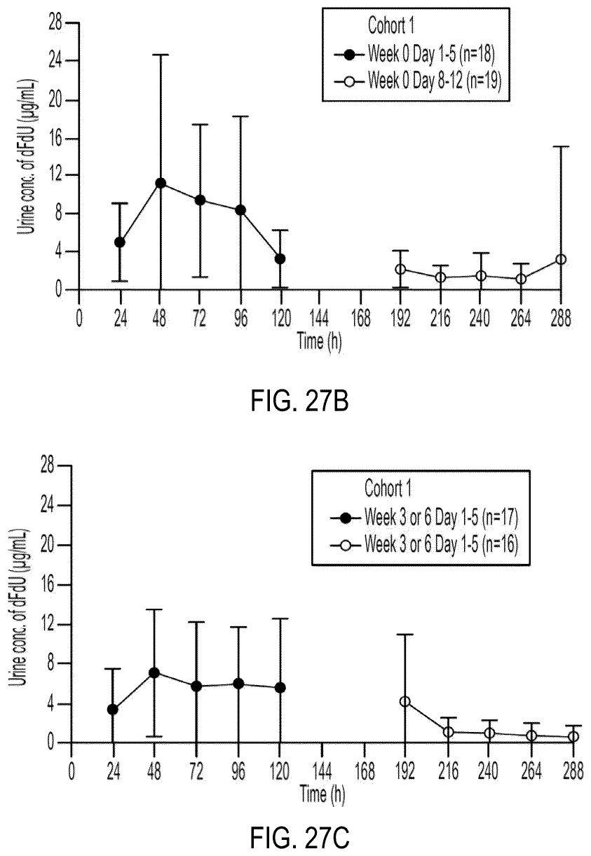

Pharmacokinetics of dFdU in urine were also measured in individuals having MIBC and receiving TAR-200 (225 mg gemcitabine; 256 mg gemcitabine HCL) in combination with intravenous cetrelimab (360 mg) for three-week dosing cycles of gemcitabine prior to radical cystectomy. Maximum dFdU concentration in urine was reached at median Tₘₐₓ of 2.5-3 days. Mean cumulative excreted dFdU amount in urine expressed as percentage of the administered dose was approximately 28.2% from 0 to 120 hours (Day 1 to Day 5 since the last dose) and 6.98% from 168 to 288 hours (Day 8 to Day 12 since the last dose). (FIGS. 27A-27C). Mean urine Cₘₐₓ of dFdU was between 10.7-15.6 μg/mL with a median tₘₐₓ of 2.5-3 days.

### Example 6: Urine Concentration-Time Profile of GRC in Individuals Following Administration of TAR-200

From the data collected in Examples 4 and 5, the total excreted gemcitabine and dFdU was further converted to GRC in mg-equivalents gemcitabine (mg-Eq. gemcitabine) and are calculated as follows: GRC (mg-Eq. gemcitabine)=Gemcitabine in mg+dFdU in mg * (263.20/264.18).

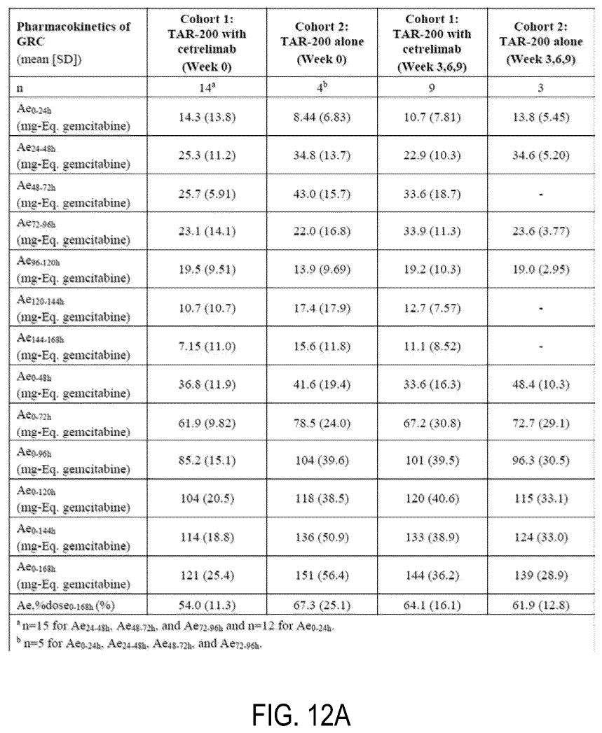

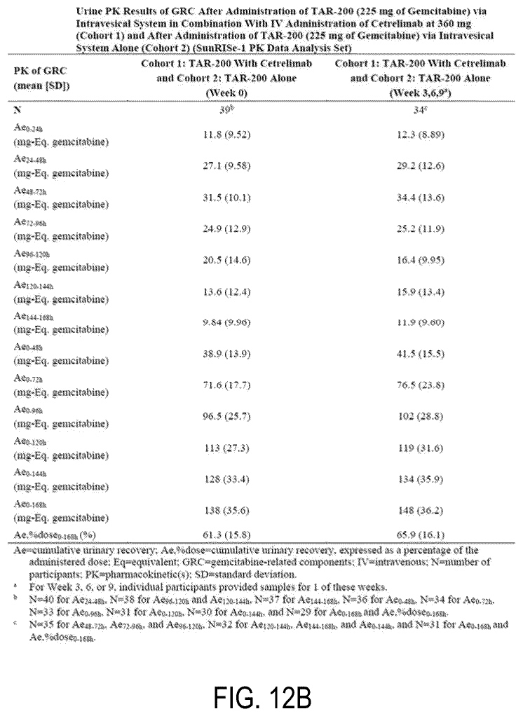

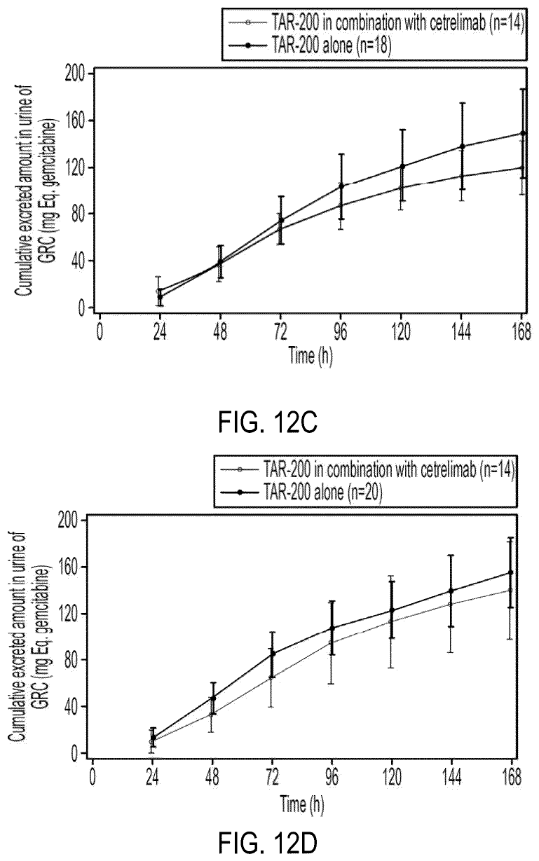

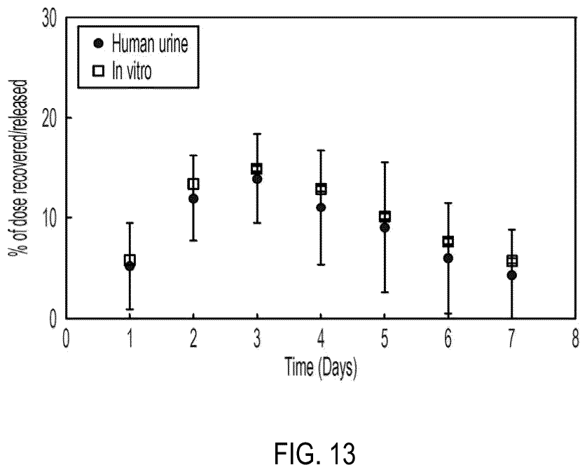

In individuals having NMIBC, mean cumulative excreted GRC amount in urine expressed as percentage of the administered dose from 0-168 hours ranged between 54.0-67.3%. (FIGS. 12A-12D). Plasma gemcitabine concentrations of all available samples were below the limit of quantitation [LOQ 0.1 μg/mL] which confirms that there is no systemic exposure of gemcitabine. Plasma dFdU concentrations of all except 2 samples were below the limit of quantitation [LOQ 0.1 μg/mL] which confirms that there is negligible systemic exposure of dFdU (≤0.397 μg/mL). Mean cumulative excreted GRC amount in urine appeared to be comparable between different dosing cycles. Mean cumulative excreted GRC amount in urine expressed as percentage of the administered dose from 0 to 168 hours was 61.3% for the dose administered on Week 0. Systemic exposure to gemcitabine and dFdU was negligible.

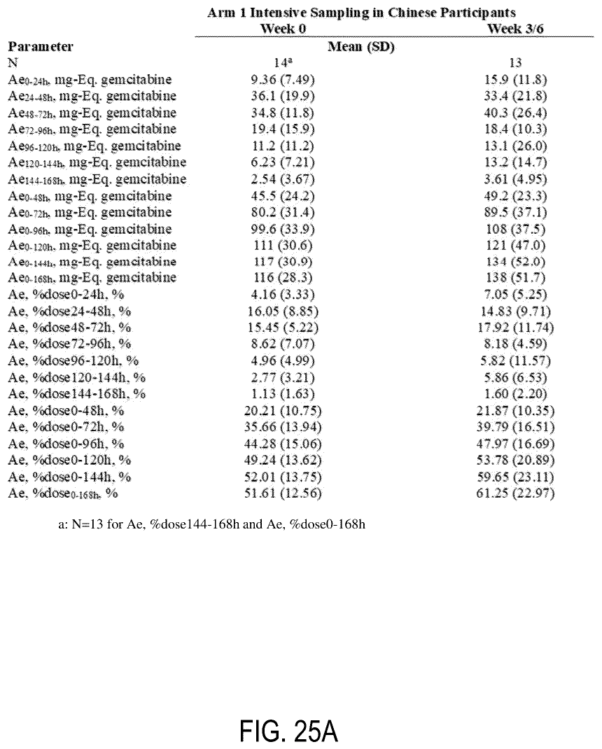

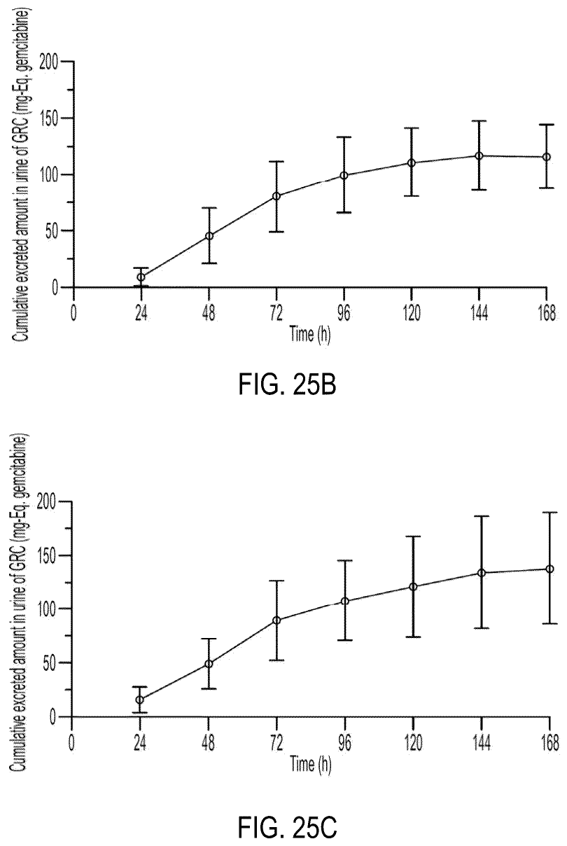

In individuals having MIBC and not scheduled from radical cystectomy, mean cumulative excreted GRC amount in urine expressed as percentage of the administered dose from 0-168 hours was approximately 51.6%. (FIGS. 25A-25C). Plasma gemcitabine concentrations of all available samples were below the limit of quantitation [LOQ 0.1 μg/mL] which confirms that there is no systemic exposure of gemcitabine. Plasma dFdU concentrations were detected in approximately 24.9% of individuals, which confirms that there is negligible systemic exposure of dFdU (≤0.397 μg/mL) in most individuals and low levels (≤1.21 μg/mL) in all individuals. Mean cumulative excreted GRC amount in urine appeared to be comparable between different dosing cycles. Mean cumulative excreted GRC amount in urine expressed as percentage of the administered dose from 0 to 168 hours was approximately 51.61% for the dose administered on Week 0.

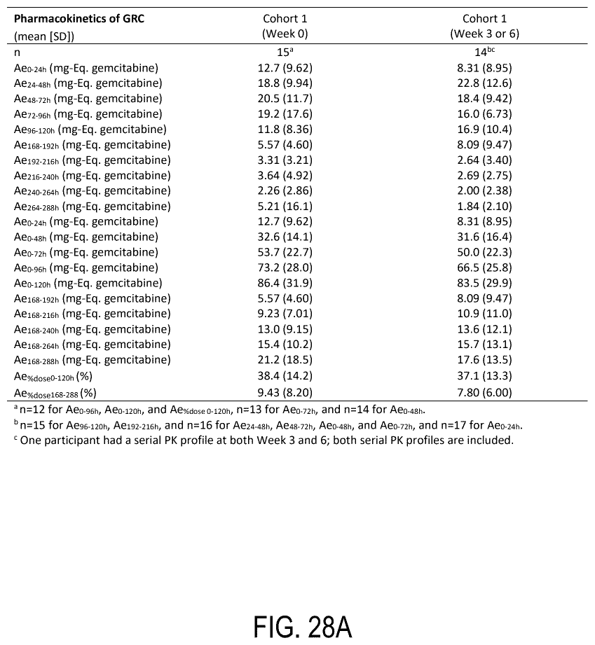

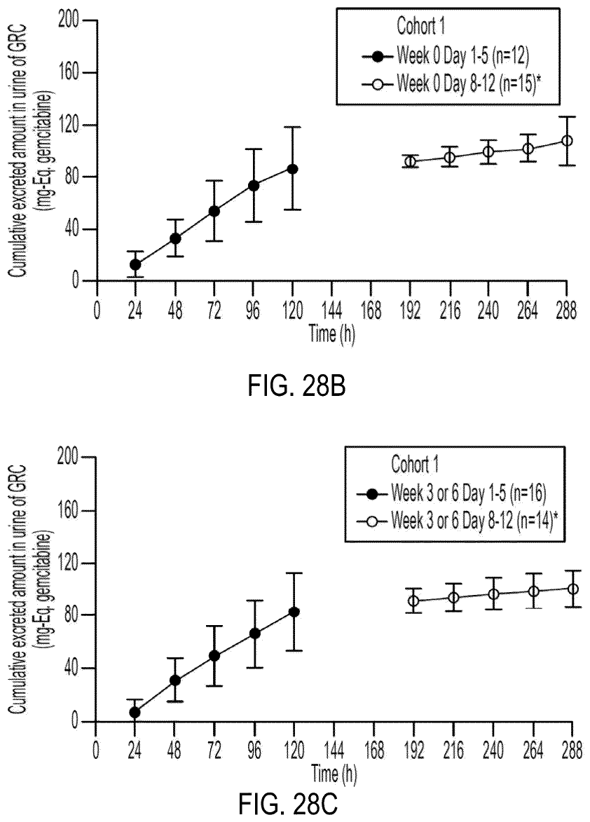

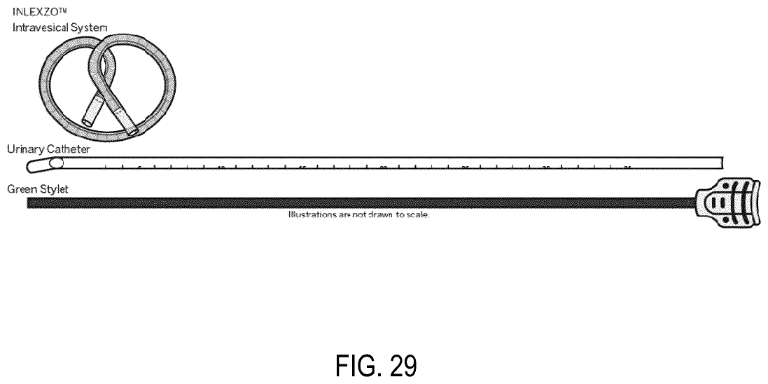

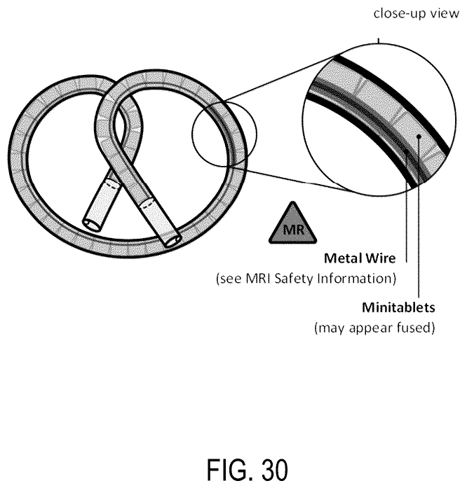

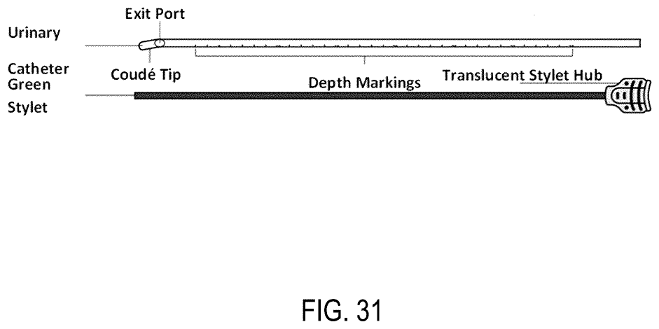

In individuals having MIBC and scheduled from radical cystectomy, mean cumulative excreted GRC amount in urine expressed as percentage of the administered dose was approximately 38.4% from Day 1 to Day 5 and 9.43% from Day 8 to Day 12. (FIGS. 28A-28C). Plasma gemcitabine concentrations of all but one sample were below the limit of quantitation [LOQ 0.1 μg/mL] which confirms that there is no systemic exposure of gemcitabine. Plasma dFdU concentrations were detected in approximately 18.6% of individuals, which confirms that there is negligible systemic exposure of dFdU (≤0.397 μg/mL) in most individuals and low levels (≤1.05 μg/mL) in all individuals. Mean cumulative excreted GRC amount in urine appeared to be comparable between different dosing cycles.

### Example 7: Development of a Population PK Model

The primary objective of the Population PK (popPK) analysis was to develop a popPK model to characterize the PK and variability of urine GRC following administration of TAR-200.

The popPK analysis was performed using pooled data from individuals treated with TAR-200 in combination with cetrelimab and individuals treated with TAR-200 monotherapy from the study described in Example 3 with a PK cutoff date of 15 Apr. 2024. In total, there were 618 PK evaluable urine samples, out of which 23 (4%) were below quantification limit (BQL) for both gemcitabine and dFdU, and 177 (29%) were BQL for either gemcitabine or dFdU. Urine samples of both analytes or either analyte being BQL were excluded from popPK modeling. The popPK analysis included 416 urine samples from 97 participants.

The model development started with a first-order release model from TAR-200 and was updated to account for the delay of drug release. Inter-individual variability (IIV) on drug release parameters were modeled assuming a log-normal distribution except for the proportion of gemcitabine available for release (BIO) parameter, which was assumed to have a logit-normal distribution to restrain individual BIO between 0 and 1. The daily GRC amount data were modeled on the logarithmic scale with an additive residual error model (equivalent to a proportional error model on the normal scale). Model selection was based on model diagnostic plots and objective function value (OFV). PK simulations were performed to derive the 7-day cumulative urine GRC amount based on individual post-hoc parameters to support exposure subgroup analysis and exposure-response (E-R) analysis. Exposure across different subgroups such as age, sex, weight, race, ethnicity, and renal function were compared using model-predicted 7-day cumulative GRC amount and observed daily urine GRC and gemcitabine exposure (ie, gemcitabine concentration, gemcitabine amount, and GRC amount).

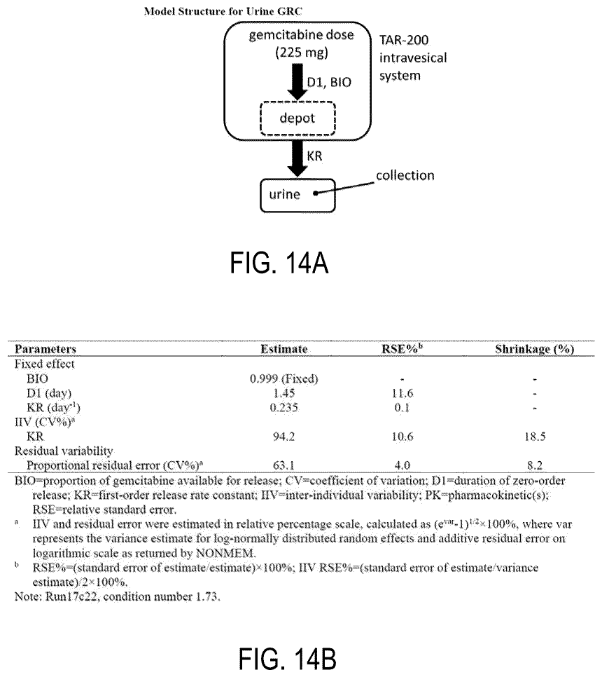

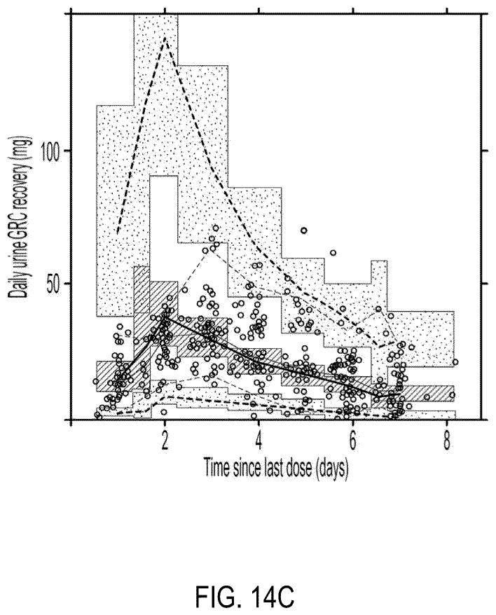

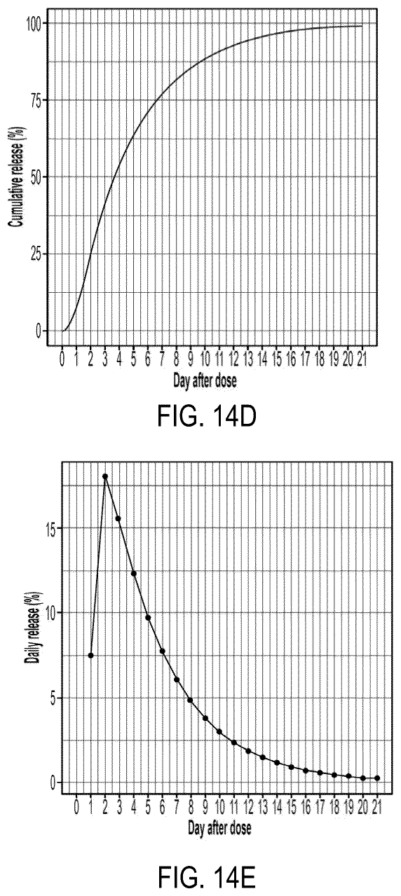

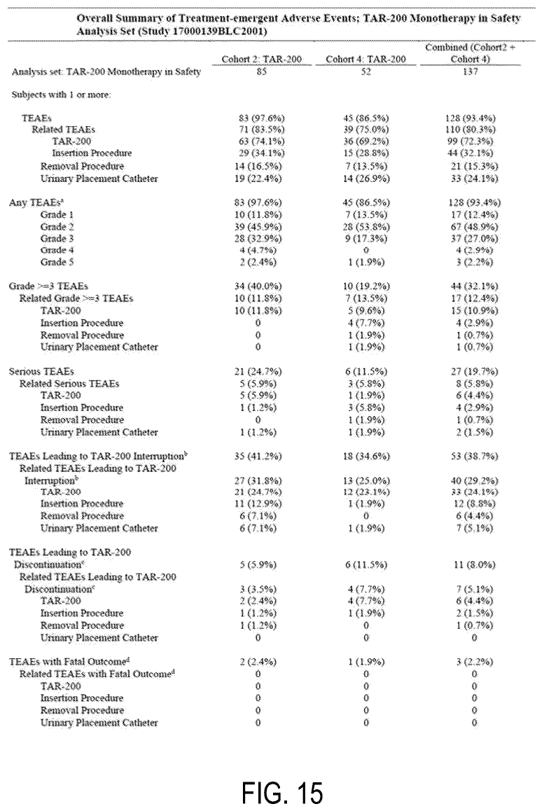

The release of gemcitabine from TAR-200 was adequately described by a sequential zero- and first-order release. The model structure was presented in FIG. 14A. The model was parameterized with proportion of gemcitabine available for release (BIO), duration of zero-order release (D1), and first-order release rate constant (KR). The parameter estimates of the final popPK model are summarized in FIG. 14B.

All model parameters were estimated with adequate precision with relative standard error (RSE)<12%. Because the study collected urine data up to Day 7 of the dosing cycle, resulting in incomplete information for the estimation of BIO, the population BIO was fixed at 0.999. This was supported by both an in vitro study and the post-use assessment of TAR-200 in minipig, which concluded that gemcitabine was nearly completely released by Day 21. IIV was quantified on KR. IIV of other release parameters such as D1, BIO was attempted but was not supported by the data (ie, model failed to converge). The residual variability was high, which could be because of incomplete collection of urine samples and/or incorrect collection time, measurement error, as well as model misspecification. The variability of urine GRC amount was largely attributed to residual unexplained variability and the IIV on KR.

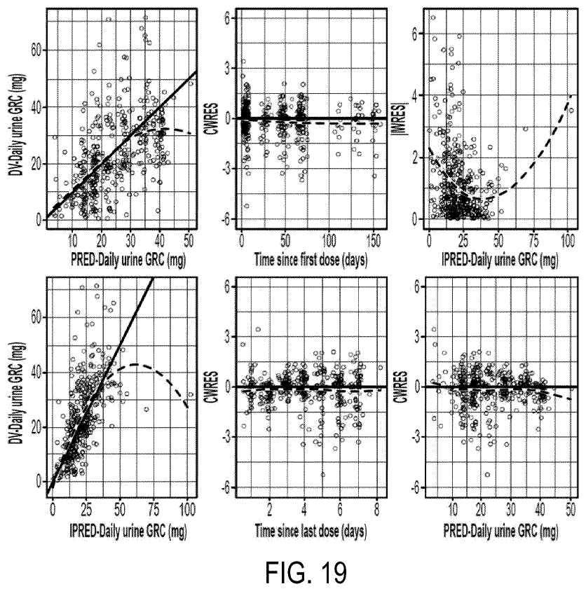

Basic goodness-of-fit (GOF) plots for the final model showed no strong bias (FIG. 19). A trend of overprediction at gemcitabine-related component (GRC)>30 mg was shown in the population prediction (PRED) versus dependent variable (DV) plot and conditional weighted residuals (CWRES) versus PRED plot, but visual predictive check (VPC) showed that the model was able to capture the central tendency of the data (FIG. 14C). Additionally, individual prediction (IPRED) agreed with DV and CWRES was distributed evenly around zero over time.

Visual predictive check (VPC) indicates that the model was able to capture the general trend of the urine GRC amount over time (FIG. 14C) and the model tended to underpredict the daily urine GRC amount. The model appeared to overpredict the overall variability of urine GRC amount.

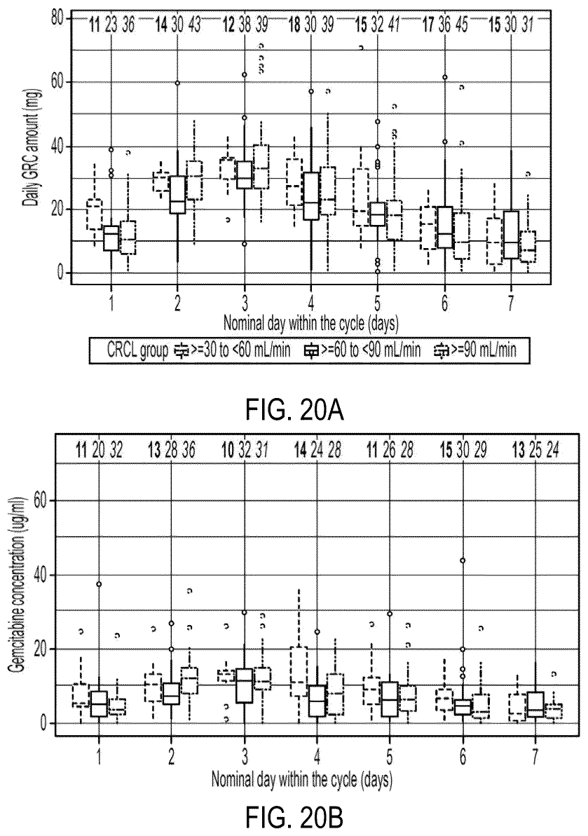

Exposure subgroup analysis was conducted using both model-predicted and observed exposure. The subgroup analysis of observed urine exposure, including GRC amount and gemcitabine concentration confirmed that urine exposure was similar across subgroups of sex, age, weight, race, ethnicity, and renal function (FIG. 20A and FIG. 20B), including mild (creatinine clearance 60 to 90 mL/min) and moderate (creatine clearance 30 to 60 mL/min) renal impairment. Urine exposure was also expected to be not affected by hepatic function as TAR-200 delivers gemcitabine locally in the bladder and results in negligible systemic exposure.

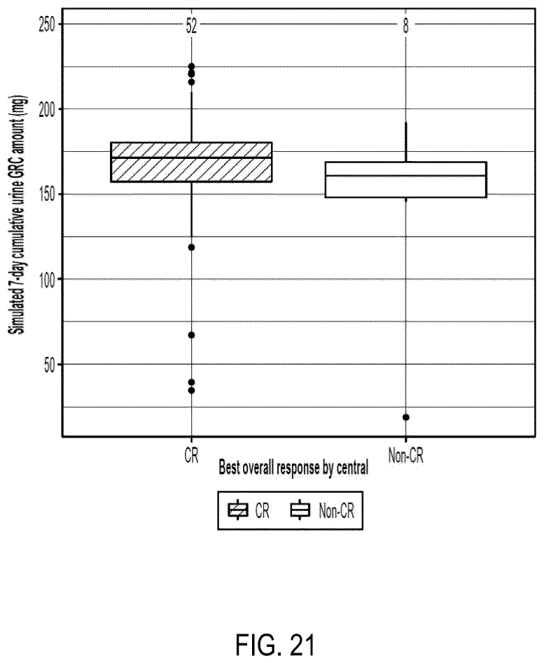

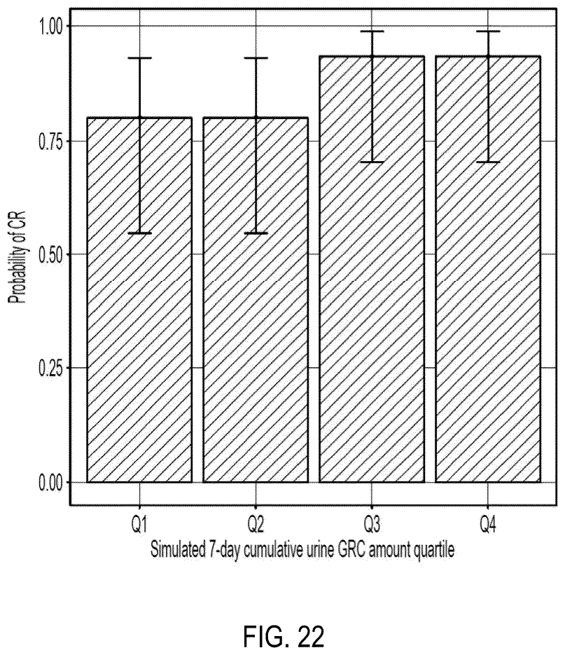

The popPK model-predicted 7-day cumulative GRC amount was comparable between participants with CR versus non-CR (FIG. 21). A barplot of probability of CR versus quartile of popPK model-predicted 7-day cumulative GRC amount (FIG. 22) also shows that CR rate was similar across different quartiles, indicating a flat exposure-response (E-R) relationship.

Based on the simulated typical profiles of cumulative release (FIG. 14D), 76.9% and 99.0% of gemcitabine were released by Day 7 and Day 21, respectively. The simulated cumulative release was consistent with the in vitro release profile (approximately 70% of gemcitabine released by Day 7, and approximately 90% by Day 21) and post-use assessment of TAR-200 in minipig, suggesting a reasonable model fit. Based on the simulated typical profile of daily release, 18.0% and 15.6% of gemcitabine was released on Day 2 and Day 3, respectively, with Day 2 being the peak release. This was generally consistent with the observed data (FIG. 14E).

### Conclusion

The release of gemcitabine from TAR-200 was characterized using a popPK model with sequential zero- and first-order release. A flat urine exposure-efficacy relationship (comparable urine GRC amount, gemcitabine concentration, and gemcitabine amount) for CR rate supports the choice of the 225 mg gemcitabine dose.

### Example 8: Update to Phase 2b Clinical Study

The following analysis of the study described in Example 3 was performed on updated data from patients treated through Mar. 31, 2025 (clinical cut-off date).

At the clinical cutoff of Mar. 31, 2025, 85 patients with BCG-unresponsive carcinoma in situ were enrolled in the TAR-200 monotherapy cohort.

The centrally confirmed complete response rate was 82.4% (95% CI, 72.6 to 89.8), with 70 of 85 patients achieving complete response (FIG. 8B). Investigator-assessed complete response rate was 83.5% (95% CI, 73.9 to 90.7), and the overall concordance between central and investigator-assessed complete response rate was 95.0%. Ninety-six percent of responses were achieved at first disease evaluation (median time to response, 2.8 months). At a 12-month response assessment, complete response rate was 45.9% (95% CI, 35.0 to 57.0) (FIG. 8C). The proportion of all patients who achieved and maintained CR for ≥12 months was 44%. In other words, 44% of patients maintained at least 12 months of DOR from the onset of CR. Subgroup analyses showed a consistently high complete response rate across all clinically relevant subgroups, including patients with and without concurrent papillary disease (82.1% and 82.5%, respectively) (FIG. 8E).

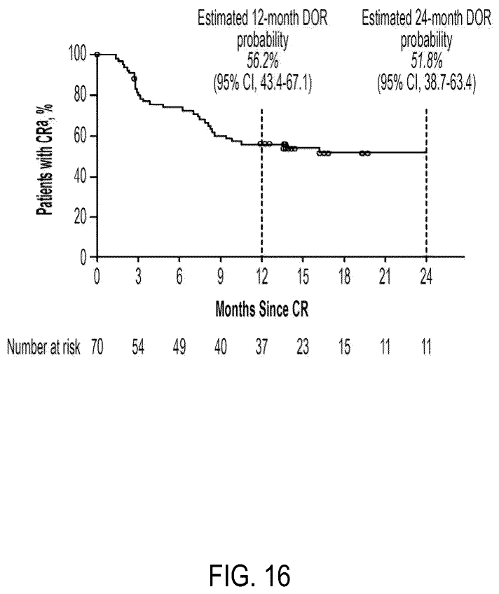

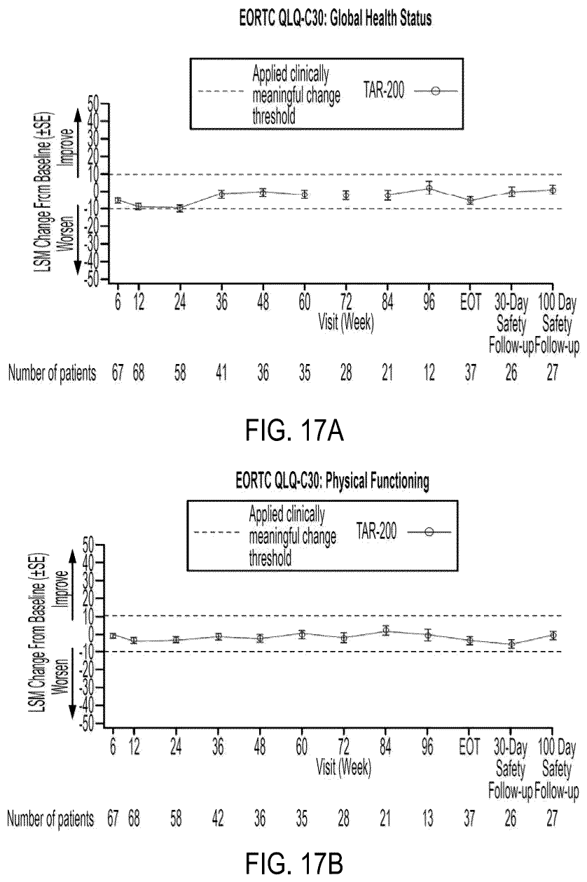

After median follow-up in responders of 20.2 months (range, 2 to 48), median duration of response was 25.8 months (95% CI, 8.3 to NE) (FIG. 9C). Twelve-month duration of response rate was 52.9%. Estimated twelve-month duration of response rate was 56.2% (95% CI, 43.4 to 67.1), and estimated 24-month duration of response rate was 51.8% (95% CI, 38.7 to 63.4) (FIG. 16). Thirty-three of 70 responders (47.1%) remained in complete response at clinical cutoff, with 11 patients having completed 2 years of treatment, 9 patients remain in response. Among responders, there were three deaths (unrelated to treatment), 23 (32.9%) had HR-NMIBC recurrence, and four (5.7%) progressed to MIBC. 25 patients received subsequent treatment, including 18 (21.2%) who underwent radical cystectomy. Median time to cystectomy was not estimable. The 12- and 24-month radical cystectomy-free rates were 86.6% (95% CI, 76.6-92.6) and 75.5% (95% CI, 63.4-84.1).

Overall survival rates at 6 and 12 months were 98.7% (95% CI, 91.2-99.8) and 94.7% (95% CI, 86.5-98.0), respectively, with seven deaths (8.2%) occurring on study. No deaths were treatment-related.

Treatment-related AEs leading to TAR-200 interruption occurred in 27 patients (31.8%), with urinary tract pain (5.9%), hematuria (4.7%), and pollakiuria (4.7%) being the most frequent TAR-200-related AEs leading to interruption. Most interruptions were limited to one to two doses, and most patients resumed treatment. Treatment-related adverse events are summarized in Table 3B below. Treatment-related adverse events of any grade occurred in 71 patients (83.5%). Insertion success rate of TAR-200 was 99% (745 of 755). The most frequent treatment-related adverse events were low-grade lower urinary tract events, including pollakiuria (43.5%), dysuria (40.0%), micturition urgency (24.7%), and urinary tract infection (21.2%). Adverse events resolved after a median of 3.0 weeks (range, 0.1+ to 150.3+). Grade ≥3 treatment-related adverse events were reported in 11 patients (12.9%), with urinary tract pain most frequent (4.7%). Treatment-related serious adverse events occurred in five patients (5.9%)—one event each of acute kidney injury, bladder pain, cystitis, cystitis pseudomonal, urinary tract infection, urinary tract pain, and urosepsis. Patients may have had ≥1 serious TRAE.

Three patients (3.5%) discontinued TAR-200 due to treatment-related adverse events. TRAEs leading to discontinuation were noninfective cystitis (n=2), bladder pain (n=1), pollakiuria (n=1), and urinary tract disorder (n=1). Patients who discontinued may have had ≥1 TRAE.

TABLE 3A

| | TAR-200 Monotherapy | | |
|---|---|---|---|
| | (N = 85) | | |
| Adverse events | Any grade | | Grade ≥3 |
| ≥1 Treatment-related adverse events | 71 | (83.5) | 11 (12.9) |
| Most frequent treatment-related adverse | | | |
| events | | | |
| Pollakiuria | 37 | (43.5) | 0 |
| Dysuria | 34 | (40.0) | 0 |
| Micturition urgency | 21 | (24.7) | 0 |
| Urinary tract infection | 19 | (22.4) | 1 (1.2) |
| Hematuria | 14 | (16.5) | 0 |
| Urinary tract pain | 9 | (10.6) | 4 (4.7) |
| Bladder pain | 7 | (8.2) | 1 (1.2) |
| Bladder spasm | 7 | (8.2) | 0 |
| Noninfective cystitis | 6 | (7.1) | 0 |
| Lower urinary tract symptoms | 5 | (5.9) | 0 |
| Urinary incontinence | 5 | (5.9) | 1 (1.2) |
| Nocturia | 4 | (4.7) | 0 |
| Urethral pain | 4 | (4.7) | 0 |
| Urinary retention | 4 | (4.7) | 1 (1.2) |
| Constipation | 3 | (3.5) | 0 |
| Cystitis | 3 | (3.5) | 1 (1.2) |
| Pelvic pain | 3 | (3.5) | 0 |
| Abdominal pain | 2 | (2.4) | 0 |
| Abdominal pain lower | 2 | (2.4) | 0 |
| Asthenia | 2 | (2.4) | 0 |
| Fatigue | 2 | (2.4) | 0 |
| Penile pain | 2 | (2.4) | 0 |
| Perineal pain | 2 | (2.4) | 0 |
| Urethral injury | 2 | (2.4) | 0 |
| Vulvovaginal pain | 2 | (2.4) | 0 |
| Treatment-related adverse events leading to | 27 (31.8%) | | |
| TAR-200 interruption | | | |
| Treatment-related adverse events leading to | 3 (3.5%) | | |
| TAR-200 discontinuation | | | |

TABLE 3B

| | TAR-200 Monotherapy | | |
|---|---|---|---|
| | (N = 85) | | |
| Adverse events | Any grade | | Grade ≥3 |
| ≥1 Treatment-related adverse events | 71 | (83.5) | 11 (12.9) |
| Most frequent treatment-related adverse | | | |
| events | | | |
| Pollakiuria | 37 | (43.5) | 0 |
| Dysuria | 34 | (40.0) | 0 |
| Micturition urgency | 21 | (24.7) | 0 |
| Urinary tract infection | 18 | (21.2) | 1 (1.2) |
| Hematuria | 14 | (16.5) | 0 |
| Urinary tract pain | 9 | (10.6) | 4 (4.7) |
| Bladder pain | 7 | (8.2) | 2 (2.4) |
| Bladder spasm | 7 | (8.2) | 0 |
| Noninfective cystitis | 6 | (7.1) | 0 |
| Lower urinary tract symptoms | 5 | (5.9) | 0 |
| Urinary incontinence | 5 | (5.9) | 0 |
| Nocturia | 4 | (4.7) | 0 |
| Urethral pain | 4 | (4.7) | 0 |
| Urinary retention | 4 | (4.7) | 1 (1.2) |
| Constipation | 3 | (3.5) | 0 |
| Cystitis | 3 | (3.5) | 1 (1.2) |
| Pelvic pain | 3 | (3.5) | 0 |
| Abdominal pain | 2 | (2.4) | 0 |
| Abdominal pain lower | 2 | (2.4) | 0 |
| Asthenia | 2 | (2.4) | 0 |
| Fatigue | 2 | (2.4) | 0 |
| Penile pain | 2 | (2.4) | 0 |
| Perineal pain | 2 | (2.4) | 0 |
| Urethral injury | 2 | (2.4) | 0 |
| Vulvovaginal pain | 2 | (2.4) | 0 |
| Treatment-related adverse events leading to | 27 (31.8%) | | |
| TAR-200 interruption | | | |
| Treatment-related adverse events leading to | 3 (3.5%) | | |
| TAR-200 discontinuation | | | |

Mean global health status (75.0 [standard deviation [SD], 16.7]) and physical functioning (86.2 [SD, 17.3]) scores were high at baseline and were maintained during treatment (did not exceed clinically meaningful change threshold of ≥10 points).

### Conclusion

These results establish TAR-200 monotherapy as the first intravesical drug-releasing system with demonstrated efficacy and safety in the treatment of localized bladder cancer. TAR-200 monotherapy provides sustained localized delivery of intravesical gemcitabine with a favorable benefit-risk profile, supporting TAR-200 as a novel bladder-sparing treatment option for patients with BCG-unresponsive high-risk NMIBC.

In particular, TAR-200 monotherapy provides the highest CR rate reported in patients with BCG-unresponsive HR NMIBC (82.4%). The onset of response was rapid without the need for reinduction (95.7% of CRs achieved at the first disease assessment) TAR-200 monotherapy responses were durable, with a median DOR of 25.8 months and 52.9% of the responses lasting for >1 year. Few responders had disease progression. The Overall health status and high physical functioning were maintained while on TAR-200 treatment and TAR-200 monotherapy was well tolerated, with serious TRAEs and TRAEs leading to discontinuation being rare.

### Example 9: Approved Drug Product Label

### Highlights of Prescribing Information

These highlights do not include all the information needed to use INLEXZO™ safely and effectively. See full prescribing information for INLEXZO™.
INLEXZO™ (gemcitabine intravesical system)
Initial U.S. Approval: 2025

### -Indications and Usage-

INLEXZO™ is a nucleoside metabolic inhibitor-containing intravesical system, indicated for the treatment of adult patients with Bacillus Calmette-Guérin (BCG)-unresponsive, non-muscle invasive bladder cancer (NMIBC) with carcinoma in situ (CIS) with or without papillary tumors. (1)

### -Dosage and Administration-

### For Intravesical Administration Only

Insert INLEXZO™ (225 mg of gemcitabine) into the bladder once every 3 weeks up to 6 months (8 doses), followed by once every 12 weeks (6 doses). (2.2)
Insert into the bladder using the co-packaged urinary catheter and stylet only. (2.1)
Remove INLEXZO™ after each 3-week indwelling period. (2.2)
See Full Prescribing Information and Instructions for Use for insertion and removal procedures. (2.3)

### -Dosage Forms and Strengths-

One single-dose 225 mg gemcitabine intravesical system (3)

### -Contraindications-

Perforation of the bladder (4, 5.1)
Prior hypersensitivity reaction to gemcitabine or any component of the product (4)

### -Warnings and Precautions-

Risks in Patients with Perforated Bladder: Evaluate the bladder before the intravesical insertion of INLEXZO™. Do not administer to patients with a perforated bladder or in whom the integrity of the bladder mucosa has been compromised. (4, 5.1)
Risk of Metastatic Bladder Cancer with Delayed Cystectomy: Delaying cystectomy can lead to the development of metastatic bladder cancer, which can be lethal. (5.2)
Magnetic Resonance Imaging (MRI) Safety: INLEXZO™ can only be safely scanned with MRI under certain conditions. (5.3)
Embryo-Fetal Toxicity: Can cause fetal harm. Advise patients of the potential risk to a fetus and to use effective contraception. (5.4, 8.1, 8.3)

### -Adverse Reactions-

The most common (>15%) adverse reactions, including laboratory abnormalities, are urinary frequency, urinary tract infection, dysuria, micturition urgency, decreased hemoglobin, increased lipase, urinary tract pain, decreased lymphocytes, hematuria, increased creatinine, increased potassium, increased AST, decreased sodium, bladder irritation, and increased ALT. (6.1)

### -Use in Specific Populations-

Lactation: Advise not to breastfeed. (8.2)
To report SUSPECTED ADVERSE REACTIONS, contact Janssen Biotech, Inc. at 1-800-526-7736 (1-800-JANSSEN) or FDA at 1-800-FDA-1088 or www.fda.gov/medwatch.
See 17 for PATIENT COUNSELING INFORMATION and FDA-approved patient labeling.
   Issued: Sepember 2025

### Full Prescribing Information: Contents*

1 INDICATIONS AND USAGE
2 DOSAGE AND ADMINISTRATION
   2.1 Important Administration Instructions
   2.2 Recommended Dosage
   2.3 Preparation and Intravesical Administration
3 DOSAGE FORMS AND STRENGTHS
4 CONTRAINDICATIONS
5 WARNINGS AND PRECAUTIONS
   5.1 Risks in Patients with Perforated Bladder
   5.2 Risk of Metastatic Bladder Cancer with Delayed Cystectomy
   5.3 Magnetic Resonance Imaging (MRI) Safety
   5.4 Embryo-Fetal Toxicity
6 ADVERSE REACTIONS
   6.1 Clinical Trials Experience
8 USE IN SPECIFIC POPULATIONS
   8.1 Pregnancy
   8.2 Lactation
   8.3 Females and Males of Reproductive Potential
   8.4 Pediatric Use
   8.5 Geriatric Use
11 DESCRIPTION
12 CLINICAL PHARMACOLOGY
   12.1 Mechanism of Action
   12.2 Pharmacodynamics
   12.3 Pharmacokinetics
13 NONCLINICAL TOXICOLOGY
   13.1 Carcinogenesis, Mutagenesis, Impairment of Fertility
14 CLINICAL STUDIES
   14.1 BCG-unresponsive NMIBC
15 REFERENCES
16 HOW SUPPLIED/STORAGE AND HANDLING
17 PATIENT COUNSELING INFORMATION
Sections or subsections omitted from the full prescribing information are not listed.

### Full Prescribing Information

### 1 Indications and Usage

INLEXZO™ is indicated for the treatment of adult patients with Bacillus Calmette-Guérin (BCG)-unresponsive, non-muscle invasive bladder cancer (NMIBC) with carcinoma in situ (CIS), with or without papillary tumors.

### 2 Dosage and Administration

### 2.1 Important Administration Instructions

Administer INLEXZO™ intravesically only. Do NOT administer by any other route. INLEXZO™ is co-packaged with a urinary catheter and stylet used to insert INLEXZO™ through the urinary catheter into the bladder. Administer using the co-packaged urinary catheter and stylet only.

INLEXZO™ should be inserted and removed by a trained healthcare provider. Healthcare providers should become thoroughly familiar with the insertion and removal instructions before attempting insertion or removal of INLEXZO™.

Prophylactic antibiotics may be used at the discretion of the treating healthcare provider with each INLEXZO™ insertion and removal.

### 2.2 Recommended Dosage

Insert INLEXZO™ (225 mg of gemcitabine) into the bladder once every 3 weeks for up to 6 months (8 doses), followed by once every 12 weeks for up to 18 months (6 doses), or until persistent or recurrent NMIBC, disease progression, or unacceptable toxicity.

Remove INLEXZO™ after each 3-week indwelling period.

### Missed Dose

If a dose is missed, it should be administered as closely as possible to the original treatment schedule.

### 2.3 Preparation and Intravesical Administration

See the Instructions for Use enclosed in the carton for complete information on preparation, intravesical administration, and removal of INLEXZO™.

INLEXZO™ is a hazardous drug. Follow applicable special handling and disposal procedures while handling INLEXZO™ and during the insertion and removal procedure.¹

Instruct patients to drink approximately 1500 mL of fluids per day during therapy with INLEXZO™ to ensure adequate urine production for drug release.

Instruct patients not to empty the bladder immediately prior to the insertion procedure. Presence of urine in the bladder can facilitate deployment of INLEXZO™. Patients can resume micturition after the insertion procedure.

During indwelling period of approximately 3 weeks, advise patients to avoid urine contact with skin, to void urine sitting on a toilet, to wash hands with soap and water and to wash their genital area with water after each urination, and to flush the toilet after use.

### 3 Dosage Forms and Strengths

One single-dose 225 mg strength gemcitabine intravesical system consisting of a flexible bi-oval shaped tube containing an almost white to light pink-brown colored component at the center surrounded by off white to light blue components.

### 4 Contraindications

INLEXZO™ is contraindicated in patients with:

Perforation of the bladder [see Warnings and Precautions (5.1)].

Prior hypersensitivity reactions to gemcitabine or any component of the product.

### 5 Warnings and Precautions

5.1 Risks in Patients with Perforated Bladder

INLEXZO™ may lead to systemic exposure to gemcitabine and to severe adverse reactions if administered to patients with a perforated bladder or to those in whom the integrity of the bladder mucosa has been compromised.

Evaluate the bladder before the intravesical administration of INLEXZO™ and do not administer to patients with a perforated bladder or mucosal compromise until bladder integrity has been restored [see Contraindications (4)].

5.2 Risk of Metastatic Bladder Cancer with Delayed Cystectomy

Delaying cystectomy in patients with BCG-unresponsive CIS could lead to development of muscle invasive or metastatic bladder cancer, which can be lethal. The risk of developing muscle invasive or metastatic bladder cancer increases the longer cystectomy is delayed in the presence of persisting CIS.

Of the 83 evaluable patients with BCG-unresponsive CIS treated with INLEXZO™ in Cohort 2 of SunRISe-1, seven patients (8%) progressed to muscle invasive (T2 or greater) bladder cancer. Three patients (3.5%) had progression determined at the time of cystectomy. The median time between determination of persistent or recurrent CIS or T1 and progression to muscle invasive disease was 94 days.

### 5.3 Magnetic Resonance Imaging (MRI) Safety

INLEXZO™ may be used with MRI only under the specific predefined conditions provided below to avoid potential safety hazards or severe adverse reactions.

Based on clinical experience in patients treated with INLEXZO™ indwelling in the bladder who underwent MRI scans and nonclinical testing, INLEXZO™ is MR Conditional. A patient with INLEXZO™ indwelling in the bladder can be scanned in an MR system under the following conditions:
Static magnetic field of 1.5-Tesla and 3-Tesla, only.
Maximum spatial gradient magnetic field of 5000 Gauss/cm or less.
Maximum magnetic resonance system reported, whole body averaged specific absorption rate of 2-W/kg for 60 minutes of continuous scanning (i.e., per pulse sequence or back-to-back sequences without breaks) in the Normal Operating Mode of operation for the MR system.

Under the scan conditions defined, INLEXZO™ is expected to produce a maximum temperature rise of 2°C after 15 minutes of continuous scanning.

In nonclinical testing, the image artifact caused by INLEXZO™ extends approximately 2 mm from INLEXZO™ using a gradient echo pulse sequence and a 3-Tesla MR system.

### 5.4 Embryo-Fetal Toxicity

Based on animal data and its mechanism of action, INLEXZO™ can cause fetal harm when administered to a pregnant woman if systemic exposure occurs [see Clinical Pharmacology (12.1)]. In animal reproduction studies, systemic administration of gemcitabine was teratogenic, embryotoxic, and fetotoxic in mice and rabbits.

Advise pregnant women and females of reproductive potential of the potential risk to a fetus. Advise females of reproductive potential to use effective contraception during treatment and for 6 months after final removal of INLEXZO™. Advise male patients with female partners of reproductive potential to use effective contraception during treatment and for 3 months after final removal of INLEXZO™ [see Use in Specific Populations (8.1, 8.3)].

### 6 Adverse Reactions

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety of INLEXZO™ monotherapy was evaluated in Cohort 2 of SunRISe-1, a multi-center, open-label study in 85 adult patients with BCG-unresponsive NMIBC with CIS, with or without papillary tumors [see Clinical Studies (14.1)].

Patients received INLEXZO™ (225 mg of gemcitabine) inserted into the bladder every 3 weeks for 6 months, followed by once every 12 weeks for up to 18 months, or until unacceptable toxicity, disease persistence, recurrence, or progression [see Dosage and Administration (2.2)].

The median number of doses of INLEXZO™ administered to patients was 9 (range: 1 to 14) doses. The median duration of exposure to INLEXZO™ was 41 weeks (range: 1 to 108 weeks).

Serious adverse reactions occurred in 24% of patients receiving INLEXZO™. Serious adverse reactions that occurred in >2% of patients included urinary tract infection, hematuria, pneumonia, and urinary tract pain. Fatal adverse reactions occurred in 1.2% of patients who received INLEXZO™, including cognitive disorder.

Permanent discontinuation of INLEXZO™ due to an adverse reaction occurred in 7% of patients. Adverse reactions which resulted in permanent discontinuation of INLEXZO™ in >1% of patients included bladder irritation, urinary frequency, cognitive disorder, hydronephrosis, and urinary tract disorder.

Dosage interruptions of INLEXZO™ due to an adverse reaction occurred in 41% of patients. Adverse reactions which required dosage interruption in >3% of patients included urinary tract infection, urinary tract pain, hematuria, urinary frequency, micturition urgency, dysuria, and genital pain.

The most common (>15%) adverse reactions, including laboratory abnormalities, were urinary frequency, urinary tract infection, dysuria, micturition urgency, decreased hemoglobin, increased lipase, urinary tract pain, decreased lymphocytes, hematuria, increased creatinine, increased potassium, increased AST, decreased sodium, bladder irritation, and increased ALT.

Table 1 summarizes the adverse reactions in SunRISe-1.

TABLE 1
Adverse Reactions Occurring in >15% of Patients in SunRISe-1

INLEXZO ™
N = 85

| | All Grades | Grade 3 |
|---|---|---|
| Adverse Reaction | % | or 4% |
| Urinary frequency | 48 | 0 |
| Urinary tract infection¹ | 44 | 6 |
| Dysuria | 42 | 0 |
| Micturition urgency¹ | 34 | 0 |
| Urinary tract pain¹ | 26 | 7 |
| Hematuria¹ | 24 | 2.4 |
| Bladder irritation¹ | 16 | 0 |

¹Includes other related terms

Other clinically significant adverse reactions (<15%) included fatigue (14%), genital pain (12%), diarrhea (11%), urinary incontinence (9%), urinary retention (7%), and nocturia (4.7%).

TABLE 2
Select Laboratory Abnormalities (>15%) That Worsened from Baseline
in Patients Who Received INLEXZO ™ in SunRISe-1

INLEXZO ™¹

| | All Grades | Grade 3 or 4 |
|---|---|---|
| Laboratory Abnormality | (%) | (%) |
| Hematology | | |
| Decreased Hemoglobin | 31 | 1.2 |
| Decreased Lymphocytes | 24 | 4.8 |
| Chemistry | | |
| Increased Lipase | 28 | 12 |
| Increased Creatinine | 24 | 0 |
| Increased Potassium | 22 | 1.2 |
| Increased AST | 17 | 1.2 |
| Decreased Sodium | 16 | 4.8 |
| Increased ALT | 16 | 1.2 |

¹The denominator used to calculate the rate varied from 82 to 83 based on the number of patients with a baseline value and at least one post-treatment value.

### 8 Use in Specific Populations

### 8.1 Pregnancy

### Risk Summary

Based on animal data and its mechanism of action, INLEXZO™ can cause fetal harm when administered to a pregnant woman if systemic exposure occurs [see Clinical Pharmacology (12.1)]. There are no available data on the use of INLEXZO™ in pregnant women to inform a drug-associated risk. In animal reproduction studies, systemic administration of gemcitabine was teratogenic, embryotoxic, and fetotoxic in mice and rabbits (see Data). Advise pregnant women and females of reproductive potential of the potential risk to a fetus.

In the U.S. general population, the estimated background risk of major birth defects and miscarriages in clinically recognized pregnancies is 2 to 4% and 15 to 20%, respectively.

### Data

### Animal Data

Gemcitabine is embryotoxic in mice. Daily systemic dosing of gemcitabine to pregnant mice increased the incidence of fetal malformation (cleft palate, incomplete ossification) at doses of 1.5 mg/kg/day. Gemcitabine was embryotoxic and fetotoxic in rabbits. Daily systemic dosing of gemcitabine to pregnant rabbits resulted in fetotoxicity (decreased fetal viability, reduced litter sizes, and developmental delays) and increased the incidence of fetal malformations (fused pulmonary artery, absence of gall bladder) at doses of 0.1 mg/kg/day.

### 8.2 Lactation

### Risk Summary

There is no information regarding the presence of gemcitabine or its metabolites in human milk, or their effects on the breastfed infant or milk production following INLEXZO™ administration. Because of the potential for serious adverse reactions in breastfed infants, advise women not to breastfeed during treatment and for 1 week after final removal of INLEXZO™.

### 8.3 Females and Males of Reproductive Potential

INLEXZO™ can cause fetal harm when administered to a pregnant woman [see Use in Specific Populations (8.1)].

### Pregnancy Testing

Verify pregnancy status in females of reproductive potential prior to initiating INLEXZO™

### Contraception

### Females

Advise females of reproductive potential to use effective contraception during treatment and for 6 months after final removal of INLEXZO™.

### Males

Because of the potential for genotoxicity, advise males with female partners of reproductive potential to use effective contraception during treatment and for 3 months after final removal of INLEXZO™ [see Nonclinical Toxicology (13.1)].

### Infertility

### Males

Based on animal studies, INLEXZO™ may impair fertility in males of reproductive potential [see Nonclinical Toxicology (13.1)]. It is not known whether these effects on fertility are reversible.

### 8.4 Pediatric Use

Safety and effectiveness of INLEXZO™ in pediatric patients have not been established.

### 8.5 Geriatric Use

Of the patients given INLEXZO™ monotherapy in Cohort 2 of SunRISe-1, 72% were 65 years of age or older and 34% were 75 years or older. There were insufficient numbers of patients <65 years of age to determine if these patients respond differently to patients 65 years of age and older.

### 11 Description

INLEXZO™ contains gemcitabine hydrochloride, a nucleoside metabolic inhibitor. Gemcitabine hydrochloride is 2′-deoxy-2′,2′-difluorocytidine monohydrochloride (β-isomer) with a molecular formula of C9H11F2N3O4·HCl, and a molecular weight of 299.66. The structural formula is:

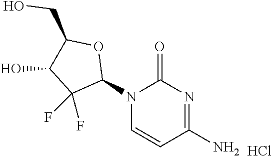 [SMILES: Nc1ccn([C@@H]2O[C@H](CO)[C@@H](O)C2(F)F)c(=O)n1 ; Cl]

INLEXZO™ is a sterile, non-resorbable intravesical system containing the equivalent of 225 mg gemcitabine (present as 256.3 mg of gemcitabine hydrochloride).

### Gemcitabine Intravesical System

INLEXZO™ is a bi-oval-shaped tube containing an almost white to light pink-brown colored gemcitabine component at the center surrounded on each side by off white to light blue-colored osmotic components.
The gemcitabine component contains 225 mg of gemcitabine and the following inactive ingredients: polyethylene glycol 8000 (8.0 mg), povidone K30 (13.4 mg), and urea (42.6 mg).
The osmotic components contain the following inactive ingredients: FD&C Blue No. 1 (0.0042 mg), polyethylene oxide 600,000 (72.0 mg), and urea (648.0 mg).

The silicone tube contains two lumens, the larger one containing the drug components and silicone spacers, and the smaller one containing a superelastic nitinol wire in a predefined shape (wireform). Both lumens are capped with a silicone adhesive. The lumen containing the gemcitabine and osmotic components has a single delivery orifice. INLEXZO™'s coiled dimensions are approximately 5.5 cm wide×4.5 cm high.

### Urinary Catheter and Stylet

INLEXZO™ is co-packaged with a sterile urinary catheter and a sterile stylet, required for transurethral insertion into the bladder. The urinary catheter and stylet are made of thermoplastic elastomer and polyethene, and consist of the following components:

A semi-transparent urinary catheter, with a rounded blunt distal tip that includes a coudé tip, a product exit port near the distal tip, and a lumen which extends from the exit port to an open proximal end. The outer diameter of the urinary catheter is 5.82 mm (17.5 Fr). Printed depth markings are placed on the urinary catheter to indicate insertion depth and orientation of the coudé tip to assist the user during insertion.

A green stylet with a hub at the proximal end is used to advance INLEXZO™ through the urinary catheter lumen and into the bladder. The stylet length and proximal hub prevent the stylet's distal end from advancement beyond the exit port.

### 12 Clinical Pharmacology

### 12.1 Mechanism of Action

Gemcitabine kills cells undergoing DNA synthesis and blocks the progression of cells through the G1/S-phase boundary. Gemcitabine is metabolized by nucleoside kinases to diphosphate (dFdCDP) and triphosphate (dFdCTP) nucleosides. Gemcitabine diphosphate inhibits ribonucleotide reductase, an enzyme responsible for catalyzing the reactions that generate deoxynucleoside triphosphates for DNA synthesis, resulting in reductions in deoxynucleotide concentrations, including dCTP. Gemcitabine triphosphate competes with dCTP for incorporation into DNA. The reduction in the intracellular concentration of dCTP by the action of the diphosphate enhances the incorporation of gemcitabine triphosphate into DNA (self-potentiation). After the gemcitabine nucleotide is incorporated into DNA, only one additional nucleotide is added to the growing DNA strands, which eventually results in the initiation of apoptotic cell death.

### 12.2 Pharmacodynamics

The exposure-response relationship and time-course of pharmacodynamic response for the safety and effectiveness of INLEXZO™ have not been fully characterized.

### 12.3 Pharmacokinetics

The systemic exposure of gemcitabine and the inactive uracil metabolite (2′-deoxy-2′,2′-difluorouridine [dFdU]), were evaluated between Days 2 and 7 during the indwelling period. The plasma gemcitabine concentrations were below the lower limit of quantification (0.1 μg/mL) in all patients at all time points. Eighteen patients (17%) had at least one quantifiable plasma dFdU concentration above the lower limit of quantification (0.1 μg/mL) and the maximum observed plasma dFdU concentration was 0.4 μg/mL. Plasma concentrations of both gemcitabine and dFdU are estimated to be less than 1% of the expected Cₘₐₓ after intravenous administration of gemcitabine.

### Excretion

Gemcitabine and dFdU are excreted in urine throughout the indwelling period for INLEXZO™ Of the total gemcitabine dose, 77% was excreted by Day 7 and 99% was excreted by Day 21 in urine as gemcitabine and dFdU.

Mean urinary excretion on Days 2 through 5 ranged from 21 to 32 mg per day excreted in the urine as gemcitabine and dFdU.

### 13 Nonclinical Toxicology

### 13.1 Carcinogenesis, Mutagenesis, Impairment of Fertility

Long-term animal studies to evaluate the carcinogenic potential of gemcitabine have not been conducted. Gemcitabine was mutagenic in an in vitro mouse lymphoma (L5178Y) Assay and was clastogenic in an in vivo mouse micronucleus assay.

Dedicated animal fertility studies have not been conducted with gemcitabine intravesical system. Gemcitabine intraperitoneal doses of 0.5 mg/kg/day in male mice resulted in moderate to severe hypospermatogenesis, decreased fertility, and decreased implantations. In female mice, fertility was not affected but maternal toxicities were observed at 1.5 mg/kg/day administered intravenously and fetotoxicity or embryo lethality were observed at 0.25 mg/kg/day administered intravenously.

### 14 Clinical Studies

### 14.1 BCG-Unresponsive NMIBC

The efficacy of INLEXZO™ was evaluated in Cohort 2 of SunRISe-1 (NCT04640623), a single-arm, multi-center trial in 83 adults with BCG-unresponsive, NMIBC with CIS, with or without papillary tumors (T1, or high-grade Ta) following transurethral resection.

BCG-unresponsive NMIBC CIS was defined as persistent or recurrent CIS alone or with Ta/T1 disease within 12 months of adequate BCG therapy. Adequate BCG therapy was defined as a minimum administration of at least five of six doses of an initial induction course plus either of: at least two of three doses of maintenance therapy or at least two of six doses of a second induction course. Prior to treatment, all patients had undergone transurethral resection of bladder tumor (TURBT) to remove all resectable disease (Ta and T1 components). Residual CIS (Tis components) not amenable to complete resection was permitted. The trial included patients who were ineligible for or who had elected not to undergo radical cystectomy and excluded patients with extra-vesical (i.e., urethra, ureter, or renal pelvis), muscle invasive (T2-T4), locally advanced, or metastatic urothelial carcinoma.

Patients received INLEXZO™ (225 mg of gemcitabine) into the bladder every 3 weeks for 6 months, followed by once every 12 weeks for up to 18 months, or until unacceptable toxicity, persistence or recurrence of CIS and/or high-grade papillary disease, or progression [see Dosage and Administration (2.2)]. Tumor status was assessed every 12 weeks during the initial two years of treatment, after which cystoscopy was performed at least every 24 weeks. Mandatory biopsies were performed 24 and 48 weeks after treatment initiation.

The major efficacy outcome measures were complete response rate at any time (defined as negative results for cystoscopy [with TURBT and centrally-reviewed biopsies as applicable] and centrally-reviewed urine cytology) and duration of response.

The median age of patients was 71 years (range: 40 to 88); 80% male; 87% White, 10% Asian, 2.4% Black or African American and 1.2% race not reported; 10% were of Hispanic or Latino ethnicity, 89% identified as not Hispanic or Latino, and 1.2% were ethnicity unknown or not reported. Baseline ECOG performance status was 0 (92%) or 1 (8%). Tumor pattern at study entry was CIS only (67%), CIS with high-grade Ta only (22%), and CIS with T1 (11%). All patients had predominant urothelial carcinoma including 1 patient (1.2%) with squamous differentiation. The median number of prior instillations of BCG was 12 (range: 7 to 42).

Efficacy results are summarized in Table 3.

TABLE 3
Efficacy Results in SunRISe-1

| | INLEXZO ™ |
|---|---|
| Endpoint | N = 83 |
| Complete Response Rate (95% CI) | 82% (72, 90) |
| Duration of Response^{a} | |
| Range in months | 0+, 44+ |
| % (n) with duration ≥ 12 months | 51% (35) |

CI= confidence interval.
^{a}Based on patients (n=68) with a complete response at any time; reflects period from the time complete response was achieved.
+Denotes ongoing response

### 15 References

1. OSHA Hazardous Drugs. Occupational Safety and Health Administration. Available at: https://www.osha.gov/hazardous-drugs (Accessed: 6 Sep. 2024).

### 16 how Supplied/Storage and Handling

### How Supplied

INLEXZO™ (gemcitabine intravesical system) contains 225 mg gemcitabine.
INLEXZO™ (NDC #57894-225-01) is available in a carton containing:
One sterile single-dose of INLEXZO™ in two clear laminate sleeves and packaged in an inner pouch. The inner pouch and a desiccant are packaged in an outer foil pouch. The outside surfaces of the inner and outer pouches are not sterile.

One sterile urinary catheter and one sterile stylet packaged together in a pouch.

### Storage

Store in the original carton at 20°C to 25°C (68°F to 77°F); with excursions permitted between 15°C to 30°C (59°F to 86°F) [see USP Controlled Room Temperature].

### Handling

INLEXZO™ is a hazardous drug. Follow applicable special handling and disposal procedures.¹

### 17 Patient Counseling Information

Advise patients to read the FDA-approved patient labeling (Patient Information).

Risk of Metastatic Bladder Cancer with Delayed Cystectomy

Inform patients that delaying cystectomy could lead to development of metastatic bladder cancer. Discuss the risk of metastatic bladder cancer and that the risk increases the longer cystectomy is delayed in the presence of persistent CIS [see Warnings and Precautions (5.2)].

### Magnetic Resonance Imaging (MRI) Safety

Inform patients that INLEXZO™ can only be safely scanned with MRI under specific conditions [see Warnings and Precautions (5.3)]. Instruct patients who will have an MRI to tell their healthcare provider that they have INLEXZO™.

This information is included in the MRI Safety Information Card. Complete the MRI Safety Information Card and give it to the patient.

### Embryo-Fetal Toxicity

Advise females of reproductive potential of the potential risk to a fetus and to inform their healthcare provider of a known or suspected pregnancy [see Warnings and Precautions (5.4) and Use in Specific Populations (8.1)].

Advise females of reproductive potential to use effective contraception during treatment and for 6 months after final removal of INLEXZO™ [see Use in Specific Populations (8.3)].

Advise males with female partners of reproductive potential to use effective contraception during treatment and for 3 months after final removal of INLEXZO™ [see Use in Specific Populations (8.3)].

### Lactation

Advise women not to breastfeed during treatment and for 1 week after final removal of INLEXZO™ [see Use in Specific Populations (8.2)].

### Infertility

Advise males of reproductive potential that INLEXZO™ may impair fertility [see Use in Specific Populations (8.3)].

### Important Post-Treatment Instructions

Instruct patients not to empty the bladder immediately prior to the insertion procedure.

Advise patients to avoid contact with urine while INLEXZO™ is indwelling in the bladder and for at least 24 hours post-removal [see Clinical Pharmacology (12.3)].

Advise patients to avoid urine contact with skin by voiding sitting on a toilet, flushing the toilet after use, and to wash hands with soap and water and to wash their genital area with water after each urination.

Advise patients to wash clothing soiled with urine promptly and separately from other clothing [see Dosage and Administration (2.3)].
Manufactured for:
Janssen Biotech, Inc.
Horsham, PA 19044, USA
U.S. License Number 1864
For patent information: www.janssenpatents.com
© Johnson & Johnson and its affiliates 2025

PATIENT INFORMATION
INLEXZO ™ (inn-lex-zo)
(gemcitabine intravesical system)

What is INLEXZO ™?
INLEXZO ™ is a prescription medicine for the treatment of adults with a type of cancer of the lining of the
bladder called non-muscle invasive bladder cancer (NMIBC), that has not spread to other parts of the body,
and that did not respond to treatment with Bacillus Calmette-Guerin (BCG).
It is not known if INLEXZO ™ is safe and effective for use in children.
Do not receive INLEXZO ™ if you:
have a tear or hole (perforation) of your bladder.
have had an allergic reaction to gemcitabine or any of the ingredients in INLEXZO ™. See the end of this
Patient Information leaflet for a complete list of ingredients in INLEXZO ™
Before receiving INLEXZO ™, tell your healthcare provider about all of your medical conditions,
including if you:
are pregnant, or plan to become pregnant. INLEXZO ™ can harm your unborn baby. You should not become
pregnant during treatment with INLEXZO ™. Tell your healthcare provider right away if you become
pregnant or think you may be pregnant during treatment with INLEXZO ™.
Females who are able to become pregnant:
Your healthcare provider will check to see if you are pregnant before starting treatment with
INLEXZO ™.
Use effective birth control (contraception) during treatment with INLEXZO ™ and for 6 months after
final removal of INLEXZO ™.
Males treated with INLEXZO ™:
If you have a female partner who is able to become pregnant, you should use effective birth control
(contraception) during treatment with INLEXZO ™ and for 3 months after final removal of
INLEXZO ™.
are breastfeeding, or plan to breastfeed. It is not known if INLEXZO ™ passes into your breastmilk. Do not
breastfeed during treatment with INLEXZO ™ and for 1 week after final removal of INLEXZO ™.
Tell your healthcare provider about all the medicines you take, including prescription and over-the-counter
medicines, vitamins, and herbal supplements.
How will I receive INLEXZO ™?
INLEXZO ™ will be inserted and removed by your healthcare provider.
INLEXZO ™ is inserted into your bladder through a tube called a urinary catheter 1 time every 3 weeks for
up to 6 months (8 doses) and then 1 time every 12 weeks for up to 18 months (6 doses).
INLEXZO ™ is removed after 3 weeks of being inserted (3-week indwelling period).
Your healthcare provider will decide how many INLEXZO ™ treatments you will receive.
Your healthcare provider may give you antibiotics before INLEXZO ™ is inserted or removed.
It is very important that you go to all of your appointments. If you miss any appointments, call your
healthcare provider as soon as possible to schedule your appointment.
Before receiving INLEXZO ™:
Do not empty your bladder right before your procedure to insert INLEXZO ™.
After receiving INLEXZO ™:
Drink about 6 to 7 cups (1500 mL) of fluids per day during treatment with INLEXZO ™ to make sure you
produce enough urine for the medicine to be released into the bladder.
You can urinate normally. There is no need to hold your urine.
Avoid contact between your skin and urine.
To urinate, males and females should sit on the toilet and flush after each use.
Wash your hands with soap and water and wash your genital area with water after each time you urinate.
Wash clothing soiled with urine right away and separately from other clothing.
After your healthcare provider removes INLEXZO ™:
Avoid contact between your skin and urine for at least 24 hours after INLEXZO ™ is removed.
What should I avoid after INLEXZO ™ is inserted?
Tell your healthcare provider that you have INLEXZO ™ before having a type of scan called Magnetic
Resonance Imaging (MRI).
INLEXZO ™ may be used under specific conditions. Your healthcare provider will give you an MRI Safety
Information Card. Keep the card in a safe place and show it to all of your healthcare providers. The card contains
important information in case you need to have an MRI. Your healthcare provider will review the information on
the MRI Safety Information Card and determine the conditions they can safely do an MRI scan while
INLEXZO ™ is in your bladder.
What are the possible side effects of INLEXZO ™?
The most common side effects of INLEXZO ™ include:

| | |
|---|---|
| frequent need to pass urine more often than usual | decreased lymphocytes |
| urinary tract infection | blood in urine |
| pain or burning sensation when passing urine | increased creatinine |
| urgent need to pass urine | increased potassium |
| decreased hemoglobin | increased aspartate aminotransferase |
| increased lipase | decreased sodium |
| pain in the urinary tract (felt in the lower stomach area | bladder irritation |
| or lower back) | increased alanine transaminase |

INLEXZO ™ may cause fertility problems in males, which may affect your ability to have children. It is
unknown if these side effects of fertility are reversible. Talk to your healthcare provider if this is a problem for
you.
Tell your healthcare provider if you have any side effect that bothers you or does not go away.
These are not all the possible side effects of INLEXZO ™.
Call your healthcare provider for medical advice about side effects. You may report side effects to FDA at 1-800-
FDA-1088.
General information about the safe and effective use of INLEXZO ™.
Medicines are sometimes prescribed for purposes other than those listed in this Patient Information leaflet. You
can ask your pharmacist or healthcare provider for information about INLEXZO ™ that is written for healthcare
professionals.
What are the ingredients in INLEXZO ™?
Active ingredient: gemcitabine hydrochloride
Inactive ingredients for gemcitabine component: polyethylene glycol 8000, povidone K30, and urea.
Inactive ingredients for osmotic components: FD&C Blue No. 1, polyethylene oxide 600,000, and urea.
Manufactured for:
Janssen Biotech, Inc.
Horsham, PA 19044, USA
U.S. License Number 1864
For patent information: www.janssenpatents.com
©Johnson & Johnson and its affiliates 2025
For more information, call 1-800-526-7736 or go to www.INLEXZO.com
This Patient Information has been approved by the U.S. Food and Drug Administration Issued: September2025

Attorney Docket No.: 76166-20036.00 Substitute Specification-Clean

### Example 10: Instructions for Use

### Instructions for Use

INLEXZO™
[inn-lex-zo]
(gemcitabine intravesical system)

### For Intravesical Administration Only

### Intended Use

INLEXZO™ releases gemcitabine into bladder urine during the indwelling period. See INLEXZO™ Prescribing Information (PI) for additional details.

The contents of the Instructions for Use provide instructions for both the insertion and removal of INLEXZO™. Ensure that the Instructions for Use are available and reviewed prior to insertion and removal of INLEXZO™.

### User

INLEXZO™ should be inserted and removed by a trained healthcare provider.

MRI Safety Information
INLEXZO ™ is MR Conditional. A patient with INLEXZO ™
may be safely scanned under the following conditions. Failure to
follow these conditions may result in injury to the patient.

| Parameter | Condition of Use/Information |
|---|---|
| Nominal Values of Static Magnetic | 1.5-Tesla and 3.0-Tesla |
| Field (T) | |
| Maximum Spatial Field Gradient | 50-T/m (5,000-gauss/cm) |
| (T/m and gauss/cm) | |
| Type of RF Excitation | Circularly Polarized (CP) (i.e., Quadrature |
| | Transmission) |
| Transmit RF Coil | Any transmit RF coil may be used. |
| Receive RF Coil | Any receive-only RF coil may be used |
| Maximum Whole Body Averaged | 2-W/kg (Normal Operating Mode) |
| SAR (W/kg) | |
| Limits on Scan Duration | Whole body averaged SAR of 2-W/kg for 60 minutes of |
| | continuous RF exposure (i.e., per pulse sequence or |
| | back-to-back sequences/series without breaks) |
| MR Image Artifact | The presence of INLEXZO ™ produces an imaging |
| | artifact. Therefore, carefully select pulse sequence |
| | parameters if INLEXZO ™ is located in the area of |
| | interest. |

### Precautions

INLEXZO™ is a hazardous drug. Follow applicable special handling and disposal procedures while handling INLEXZO™ and during the insertion and removal procedure. Dispose of the used urinary catheter and stylet, INLEXZO™, and its packaging per facility procedures and per applicable federal, state, and local regulations.

Wear gloves, and take appropriate precautions, per local guidelines for handling hazardous drugs, to prevent skin or mucus membrane exposure while handling INLEXZO™ and during the insertion and removal procedure.

If contact with INLEXZO™ is suspected, immediately wash the skin thoroughly or rinse the mucosa with copious amounts of water.

Advise patients and caregivers to exercise caution when handling urine during indwelling period of approximately 3 weeks. See INLEXZO™ PI for details.

INLEXZO™ contains one single-dose 225 mg strength gemcitabine intravesical system consisting of a flexible bi-oval shaped tube containing an almost white to light pink-brown colored component at the center surrounded by off white to light blue osmotic components.

### Important Information

Use aseptic technique during insertion and removal of INLEXZO™.

Follow these instructions carefully, to avoid patient injury and ensure proper functioning.

### Insertion

To ensure proper insertion of INLEXZO™ and to avoid damage to INLEXZO™, use only the following:
Water-based lubricant
Urinary catheter and stylet (supplied)

Do not use the urinary catheter and stylet for any other purpose. Do not re-sterilize/re-use the urinary catheter or stylet. Re-use of the urinary catheter and stylet can lead to its degradation, failure, and contamination, which can increase the risk of infection or transmission of blood borne pathogens to patients and users.

Do not use any components that are damaged or have damaged packaging.

Check the expiration (‘EXP’) date before use.

Do not use INLEXZO™ if expiration date has passed.

### Removal

To ensure proper INLEXZO™ removal and to avoid damage to INLEXZO™ and/or cystoscope, use only the following:
Non-cutting, grasping forceps
Flexible or rigid cystoscope

### Storage

Store in the original package at 20°C to 25°C (68°F to 77°F); with excursions permitted between 15°C to 30°C (59°F to 86°F) [see USP Controlled Room Temperature].

INLEXZO™

### Intravesical System

### Urinary Catheter and Stylet

Prepare components for INLEXZO ™ insertion

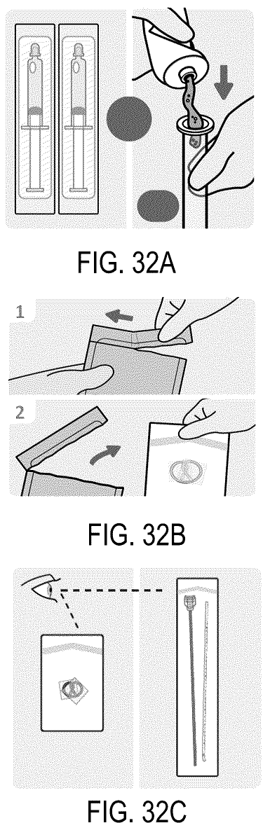

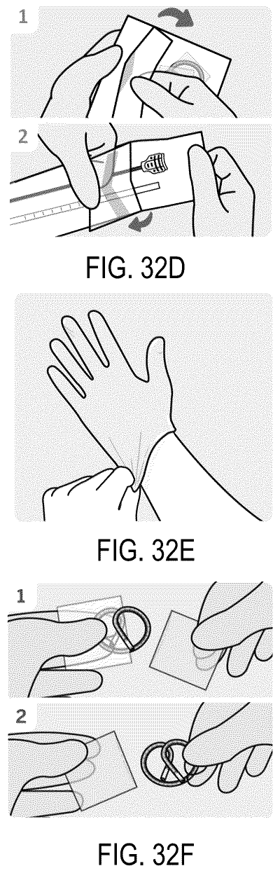

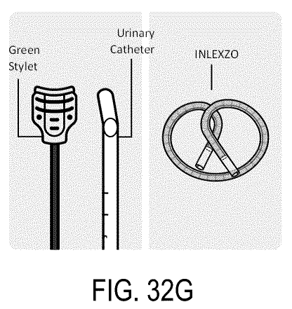

1. Gather supplies
Included in the product carton:
One sterile INLEXZO ™
One sterile urinary catheter and one sterile stylet
Not included in the product carton:
Multiple pairs of gloves
Two 10 mL prefilled water-based lubricant syringes
OR
Two empty 10 mL syringes and water-based lubricant
2. Put on gloves
3. Prepare two syringes with sterile water-based lubricant (FIG. 32A)
Remove each sterile syringe prefilled with water-based lubricant from its packaging and place
each syringe on the sterile work surface using aseptic technique.
OR
Fill each of the two empty syringes with 2 mL to 3 mL of water-based lubricant plus additional
lubricant for urinary catheter tip lubrication.
Place each lubricant syringe on a sterile work surface using aseptic technique.
The lubricant is to be used to lubricate the tip of the urinary catheter and to facilitate the insertion
of INLEXZO ™.
4. Open the INLEXZO ™ foil pouch (FIG. 32B)
1. Tear open the INLEXZO ™ outer foil pouch at tear notch.
2. Remove the white INLEXZO ™ inner pouch.
5. Examine the white component pouches for damage (FIG. 32C)
Check the white INLEXZO ™ pouch and white pouch containing the urinary catheter and stylet for
damage (e.g., cuts, tears, punctures) that could compromise sterility of the components before
opening.
Do not use if packaging is damaged.
Surfaces of the white INLEXZO ™ pouch and the white component pouches are not sterile.
6. Open the white component pouches and transfer the contents onto a sterile
work surface (FIG. 32D)
1. Open the white INLEXZO ™ pouch and transfer INLEXZO ™ onto a sterile work surface.
2. Do not remove plastic sleeves from INLEXZO ™.
3. Open the white pouch containing the urinary catheter and stylet and transfer the contents onto
a sterile work surface.
7. Put on sterile gloves (FIG. 32E)
1. Ensure your patient is prepared for the procedure.
2. Put on sterile gloves.
8. Remove plastic sleeves (FIG. 32F)
Remove the plastic sleeves from INLEXZO ™
9. Inspect components (FIG. 32G)
Inspect INLEXZO ™, the urinary catheter, and green stylet for damage.
Do not use if the urinary catheter or green stylet are damaged or if the outer surface of INLEXZO ™
is damaged.

Insert INLEXZO ™

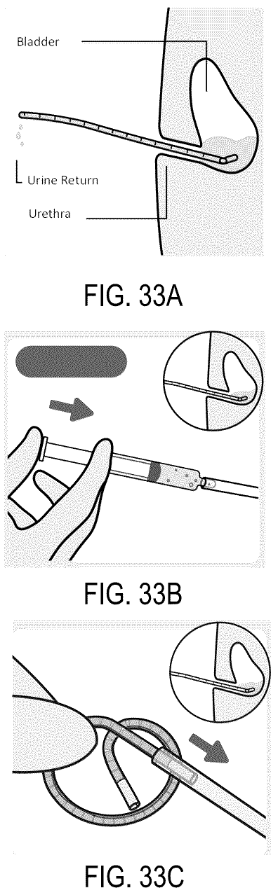

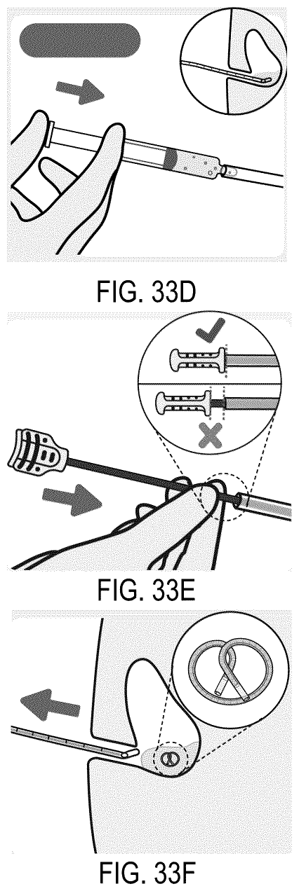

1. Lubricate the urinary catheter
Lubricate tip of the urinary catheter.
The urinary catheter should be free of INLEXZO ™ and the stylet.
2. Insert the urinary catheter (without stylet) (FIG. 33A)
Introduce the urinary catheter into the urethra by hand and advance until urine return. Do not empty
the bladder.
Use depth markings to maintain coude tip orientation and insertion depth position throughout the
procedure.
Do not excessively force the urinary catheter into the bladder.
In case of resistance, careful assessment and standard procedural techniques can be
performed, as appropriate, in order to safely advance the urinary catheter into the bladder.
If advancement is obstructed or cause cannot be determined or resolved, withdraw the
urinary catheter to avoid patient injury or urinary catheter damage.
Do not re-use urinary catheter.
3. Inject lubricant from the first syringe into the urinary catheter (FIG. 33B)
Inject 2 mL to 3 mL of lubricant from the first syringe into the end of the urinary catheter with the
urinary catheter placed in the bladder.
4. Insert INLEXZO ™ into the urinary catheter (FIG. 33C)
Insert either end of INLEXZO ™ into the urinary catheter and advance until fully inserted.
5. Inject lubricant from the second syringe into the urinary catheter (FIG. 33D)
Inject 2 mL to 3 mL of lubricant from the second syringe into the urinary catheter to help advance
INLEXZO ™ further, with the urinary catheter placed in the bladder.
6. Insert stylet into the urinary catheter_(FIG. 33E)
Slowly insert the stylet into the urinary catheter until the stylet hub is flush with end of the urinary
catheter. This ensures INLEXZO ™ exits the urinary catheter and enters the bladder.
If INLEXZO ™ cannot be advanced, remove the urinary catheter and stylet together as a
single unit.
Ensure INLEXZO ™ is also removed.
Do not attempt to re-use the removed INLEXZO ™.
Begin again by obtaining a new carton of INLEXZO ™ including a new urinary catheter and
stylet.
7. Remove the urinary catheter and stylet together as a single unit (FIG. 33F)
Do not remove the urinary catheter and stylet individually.
INLEXZO ™ should remain inside the bladder.
Retain Instructions for Use for the removal procedure.

Post-Insertion Instructions

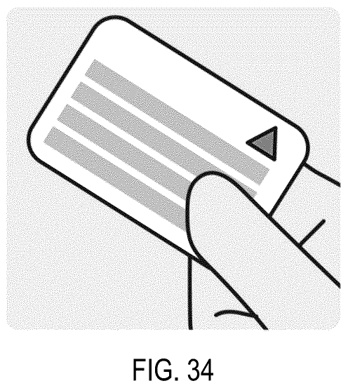

1. Provide the completed MRI Safety Information Card to the patient (FIG. 34)
Remove the MRI Safety Information Card from the carton.
Complete the details and give it to the patient.
Advise the patient to carry the card and to show their current and future healthcare providers in case
of need for MRI scans.
INLEXZO ™ contains a metal wire. The patient can safely undergo an MR exam only under very
specific conditions (see MRI Safety Information).
2. Inform the patient and caregivers about the indwelling period
The indwelling period is approximately 3 weeks. See INLEXZO ™ PI for additional information.
Inform the patient and caregivers that INLEXZO ™ will remain in the bladder for the indwelling
dosing period.
INLEXZO ™ contains a hazardous drug. The patient and caregivers should be made aware of the
need to exercise caution when handling urine during the indwelling period. See INLEXZO ™ PI
for additional information.

Removal of INLEXZO ™ after the indwelling period

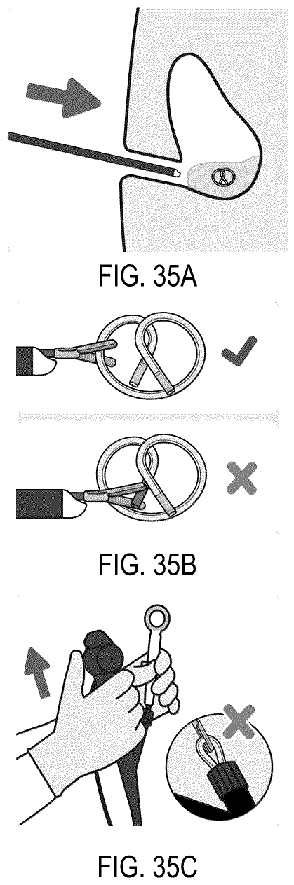

1. Lubricate the cystoscope
Use a water-based lubricant to lubricate the cystoscope.
2. Insert the cystoscope (FIG. 35A)
Insert the cystoscope into the bladder to locate INLEXZO ™.
3. Grasp INLEXZO ™ (FIG. 35B)
Introduce non-cutting grasping forceps through the working channel of the cystoscope.
Do not use cutting forceps.
Grasp INLEXZO ™ over tubing and metal wire.
Do not grasp on or near the ends of INLEXZO ™.
Grasping near the ends of INLEXZO ™ could result in exposing the metal wire, potentially causing
damage to surrounding tissue.
4. Remove INLEXZO ™ (FIG. 35C)
Remove the cystoscope and forceps out of the urethra together to remove INLEXZO ™ under
direct vision.
Do not remove INLEXZO ™ through working channel of the cystoscope. Doing so may damage
INLEXZO ™ and/or the cystoscope.
5. Inspect INLEXZO ™
After removal, inspect INLEXZO ™ to confirm it is intact and unbroken.

Manufactured for:
Janssen Biotech, Inc.
Horsham, PA 19044, USA.
U.S. License Number 1864

This Instructions for Use has been approved by the U.S. Food and Drug Administration. Approved: September 2025

For patent information:
www.janssenpatents.com
© Johnson & Johnson and its affiliates 2025

### Example 11: Further Update to Phase 2b Clinical Study

The following analysis of the study described in Example 3 was performed on updated data from patients treated through Jan. 13, 2026 (clinical cut-off date).

At the clinical cutoff of Jan. 13, 2026, 85 patients with BCG-unresponsive carcinoma in situ were enrolled in the TAR-200 monotherapy cohort.

The centrally confirmed complete response rate was 82.4% (95% CI, 72.6 to 89.8). After median follow-up in responders of 29.7 months (range, 5 to 58), median duration of response was 17.7 months (95% CI, 8.3 to 26.5). The 24-month duration of response rate was 41.6% (95% CI, 28.9 to 53.8). Thirty-one of 70 responders (44.3%) remained in complete response at clinical cutoff. 58 of 70 responders completed 24 months of follow-up. 39 of 70 (55.7%) had disease recurrence (35.7%) or progression (5.7%). 24-month cystectomy-free rate was 73.7% (95% CI, 61.8-82.4).

### Conclusion

These results further support the establishment of TAR-200 monotherapy as the first intravesical drug-releasing system with demonstrated efficacy and safety in the treatment of localized bladder cancer.

1-154. (canceled)

155. A method of treating bladder cancer in an individual comprising inserting an intravesical system comprising about 225 mg of gemcitabine into the bladder of the individual once about every three weeks for at least 24 weeks,
wherein each intravesical system is removed from the bladder about three weeks after insertion into the bladder and wherein each intravesical system is removed prior to insertion of another intravesical system,
wherein each intravesical system provides sustained release of gemcitabine into bladder urine to target a pharmacokinetic (PK) profile for gemcitabine, wherein the PK profile has a mean urinary excretion on Days 2 through 5 of 21 mg to 32 mg per day as gemcitabine and dFdU,
wherein about 65% to about 85% of the gemcitabine is released over about the first 7 days after insertion into the bladder and about 85% to about 100% of the gemcitabine is released over about 21 days after insertion into the bladder, and
wherein the intravesical system comprises gemcitabine minitablets comprising a total of:
i) about 225 mg gemcitabine;
ii) about 10 mg to about 20 mg Povidone 30;
iii) about 5 mg to about 12 mg polyethylene glycol (PEG) 8000; and
iv) about 35 mg to about 50 mg urea.

156. The method of claim 155, wherein about 77% of the gemcitabine is released over about the first 7 days after insertion into the bladder and about 99% of the gemcitabine is released over about 21 days after insertion into the bladder, and
wherein the percent of gemcitabine released is estimated using in vivo data comprising urine gemcitabine related component (GRC) amount-time profiles, and wherein the percent of gemcitabine released is estimated using nonlinear mixed effect modeling.

157. The method of claim 155, wherein the PK profile has:
a) a mean Cₘₐₓ of gemcitabine of about 10 μg/mL to about 25 μg/mL;
b) a Tₘₐₓ of gemcitabine of about 2 days to about 4 days; and/or
c) a mean cumulative excreted gemcitabine of about 30% to about 60%, wherein the mean cumulative excreted gemcitabine is a percentage of the administered dose.

158. The method of claim 155, wherein the individual has non-muscle invasive bladder cancer (NMIBC).

159. The method of claim 155, wherein each intravesical system produces a systemic exposure of gemcitabine in the individual of less than about 0.1 μg/mL.

160. The method of claim 155, wherein each intravesical system produces a maximum mean amount of about 31.5 mg gemcitabine related components (GRC) in urine on Day 3.

161. The method of claim 155, wherein each intravesical system releases about 2% to about 20% of the gemcitabine on each of days 1-7 after the intravesical system is inserted into the bladder of the individual.

162. The method of claim 155, wherein each intravesical system produces an average urine concentration of about 2 μg/mL to about 15 μg/mL on about days 1-7 after insertion into the bladder of the individual.

163. The method of claim 155, wherein each gemcitabine minitablet comprises:
i) about 19 mg to about 24 mg gemcitabine HCL;
ii) about 0.5 mg to about 1.5 mg Povidone 30;
iii) about 0.4 mg to about 1 mg polyethylene glycol (PEG) 8000; and
iv) about 2 mg to about 5 mg urea.

164. The method of claim 155, wherein each intravesical system comprises osmotic minitablets comprising:
i) about 15 mg to about 25 mg urea; and
ii) about 1 mg to about 4 mg polyethylene oxide (PEO).

165. The method of claim 155, wherein each intravesical system comprises a flexible bi-oval shaped tube.

166. The method of claim 155, wherein when in a retention shape, the intravesical system defines a bi-oval coiled shape having a maximum width and a maximum height perpendicular to the maximum width, wherein the maximum width is about 4-7 cm and the maximum height is about 3-6 cm.

167. The method of claim 155, wherein each intravesical system comprises 12 gemcitabine minitablets and 30 osmotic agent minitablets.

168. The method of claim 155, wherein each intravesical system comprises:
a) 12 gemcitabine minitablets comprising a total of:
i) about 225 mg gemcitabine;
ii) about 13.4 mg Povidone 30;
iii) about 8 mg polyethylene glycol (PEG) 8000; and
iv) about 42.6 mg urea; and
b) 30 osmotic agent minitablets comprising a total of:
i) about 648 mg urea; and
ii) about 72 mg polyethylene oxide (PEO).

169. The method of claim 155, wherein each intravesical system comprises an orifice with an about 25 micron to about 300 micron diameter.

170. The method of claim 155, wherein the individual is ineligible for or has elected not to undergo radical cystectomy.

171. The method of claim 155, wherein such treatment results in
a) a complete response (CR) rate of about 72% to about 90% in a population of patients who have received such treatment;
b) a median duration of response (DoR) of about 9 months to about 30 months in a population of patients who have received such treatment;
c) a 12-month complete response (CR) rate of about 40% to about 50% in a population of patients who have received such treatment; and/or
d) a 12-month duration of response (DoR) rate of about 43% to about 67% in a population of patients who have received such treatment.

172. The method of claim 171, wherein such treatment results in
a) a complete response (CR) rate of about 82% in a population of patients who have received such treatment;
b) a median duration of response (DoR) of about 25.8 months in a population of patients who have received such treatment;
c) a 12-month complete response (CR) rate of about 45.9% in a population of patients who have received such treatment; and/or
d) a 12-month duration of response (DoR) rate of about 51% in a population of patients who have received such treatment.

173. The method of claim 155, wherein each intravesical system produces a mean urinary excretion on Days 2 through 5 of 21 mg to 32 mg per day as gemcitabine and dFdU.

174. The method of claim 155, wherein each intravesical system comprises an almost white to light pink-brown colored gemcitabine component at the center surrounded on each side by off white to light blue-colored osmotic components.

175. The method of claim 155, wherein such treatment results in:
a) a 24-month duration of response (DoR) rate of about 28% to about 54% in a population of patients who have received such treatment; and/or
b) a 24-month cystectomy-free rate of about 61% to about 83% in a population of patients who have received such treatment.

176. The method of claim 155, wherein the about 225 mg of gemcitabine comprises about 256.3 mg of gemcitabine HCL.

177. A method of treating Bacillus Calmette Guerin (BCG)-unresponsive, non-muscle invasive bladder cancer (NMIBC) with carcinoma in situ (CIS) with or without papillary tumors in an individual, the method comprising inserting an intravesical system comprising about 225 mg of gemcitabine into the bladder once every 3 weeks up to 6 months (8 doses), followed by once every 12 weeks for up to 18 months, or until persistent or recurrent NMIBC, disease progression, or unacceptable toxicity.

178. The method of claim 177, comprising a total of 14 doses over 2 years of therapy.

179. An intravesical system comprising gemcitabine minitablets comprising a total of:
i) about 225 mg gemcitabine;
ii) about 10 mg to about 20 mg Povidone 30;
iii) about 5 mg to about 12 mg polyethylene glycol (PEG) 8000; and
iv) about 35 mg to about 50 mg urea,
wherein about 77% of the total gemcitabine dose in the intravesical system is released into the urine as gemcitabine and dFdU over about the first 7 days after insertion into the bladder, and about 99% of the total gemcitabine dose in the intravesical system is released into the urine as gemcitabine and dFdU over about the first 21 days after insertion into the bladder.

180. A kit comprising an intravesical system and instructions for use to treat Bacillus Calmette Guerin (BCG)-unresponsive, non-muscle invasive bladder cancer (NMIBC) with carcinoma in situ (CIS) with or without papillary tumors in an individual, the intravesical system comprising
a housing configured for intravesical insertion, the housing defining a drug reservoir lumen and a retention frame lumen, the drug reservoir lumen having a release orifice with an about 150 micron diameter;
a retention frame comprising a nitinol wire located in the retention frame lumen;
12 gemcitabine minitablets located within the drug reservoir lumen, the 12 gemcitabine minitablets comprising a total of about 225 mg gemcitabine;
30 osmotic minitablets located within the drug reservoir lumen, with 15 of the 30 osmotic minitablets located on a first side of the 12 gemcitabine minitablets and with the remaining 15 of the 30 osmotic minitablets located on a second, opposite side of the 12 gemcitabine minitablets;
wherein the gemcitabine minitablets are located closer to the release orifice than the osmotic minitablets;
wherein the housing is configured to release gemcitabine from the drug reservoir lumen through the release orifice via osmotic pressure generated by the osmotic minitablets;
wherein the housing is elastically deformable between a retention shape configured to retain the system in the individual's bladder and a deployment shape configured for passage of the system through the individual's urethra.

181. The kit of claim 180, further comprising a urinary placement catheter adapted for insertion of the intravesical system into the bladder.
